(19) United States
(12) Patent Application Publication
(10) Pub. No.: US 2022/0233244 A1
(43) Pub. Date: Jul. 28, 2022

(54) AUDIO AUGMENTED REALITY CUES TO FOCUS ON AUDIBLE INFORMATION

(71) Applicant: ETHICON LLC, Guaynabo, PR (US)

(72) Inventors: Frederick E. Shelton, IV, Hillsboro, OH (US); Kevin M. Fiebig, Cincinnati, OH (US); Jason L. Harris, Lebanon, OH (US)

(21) Appl. No.: 17/156,329

(22) Filed: Jan. 22, 2021

(51) Int. Cl. A61B 34/20; G06T 19/00; G06F 3/01; H04R 5/04

(52) U.S. Cl. CPC A61B 34/20; G06T 19/006; G06T 2210/41; H04R 5/04; G06F 3/011

(57) ABSTRACT

An audible augmented reality (AR) computing system may receive audio data such as audible information, from a sensing system in an operating room (OR), ambient noise in the OR. The audio AR computing system may generate AR content. For example, the audio AR computing system may generate AR content based on the audible data. The AR content may be or may include audible information associated with the audio data. The audio AR computing system may receive an adjustment indication. The adjustment indication may be or may include one or more of adjustment information for the AR content. For example, the adjustment indication may be or may include one or more of a surgical task indication, a task importance indication, an audio insertion indication, an audio translation indication, an audio source location indication. The audio AR computing system may adjust the AR content based on the received adjustment indication.

### CROSS-REFERENCE TO RELATED APPLICATIONS

This application is related to the following, filed contemporaneously, the contents of each of which are incorporated by reference herein:
Attorney Docket No. END9290USNP1, titled METHOD OF ADJUSTING A SURGICAL PARAMETER BASED ON BIOMARKFR.

### BACKGROUND

Surgical procedures are typically performed in surgical operating theaters or rooms in a healthcare facility such as a hospital. Various surgical devices and systems and/or sensing systems are utilized in performance of a surgical procedure. In the digital and information age, health care professionals may utilize technologies to improve patient practices. It would be desirable to find ways to utilize augmented reality (AR) computing systems, such as audio AR computing systems and/or visual AR computing systems, to aid health care professionals to imp rove patient practices.

### SUMMARY

An audible augmented reality (AR) computing system may receive audio data, such as audible information, from a sensing system in an operating room (OR). In examples, the audio data may be or may include measurement data associated with a user. A user may be or may include a health care professional (HCP), such as a surgeon, or a patient. In examples, the audio data may be or may include ambient noise of the OR. In examples, the audio data may block and/or cancel the ambient noise of the OR. In examples, the audio data may be or may include music, such as calming music, audible feedback information, audible information associated with a surgical step and/or task, and/or other audible information associated with a surgical procedure.

The audio AR computing system may generate AR content. For example, the audio AR computing system may generate the AR content based on the audible data. The AR content may be or may include audible information associated with the audio data. The AR content may be or may include audible AR information that is to be transmitted to a user who is wearing the audio AR computing system and/or a headset controlled by the audio AR computing system. For example, the audible AR computing system may be or may include a surgeon sensing system and/or patient sensing system.

The audio AR computing system may obtain an adjustment indication. The adjustment indication may be or may include adjustment information for the AR content. For example, the adjustment indication may be or may include one or more of a surgical task indication, a task importance indication, an audio insertion indication, an audio translation indication, an audio source location indication. The surgical task indication may indicate a surgical task being performed or to be performed. The task importance indication may indicate an importance of the surgical task. The audio insertion indication may indicate a calming audio and/or music insert. The audio translation indication may indicate an audio translation of the audio data. The audio source location indication may indicate an audio source location of the audio data. For example, the adjustment indication may be obtained (e.g., received) from a surgical computing system, such as a surgical hub.

The audio AR computing system may adjust the generated AR content, e.g., based on the adjustment indication. In examples, the audio AR computing system may adjust the generated AR content based on the adjustment indication indicating an importance of a surgical step. The audio AR computing system may identify an audio AR setting associated with the importance of the surgical step. The audio AR setting may include a volume associated with the audible information (e.g., that is associated with the surgical step), a frequency of transmission of the audible information, and/or a voice associated with the audible information. The audio AR computing system may adjust the generated AR content in accordance with the audio AR setting. For example, the audio AR computing system may adjust the volume of the AR content based on the AR setting. The audio AR computing system may increase or decrease the volume of the AR content based on the AR setting. The audio AR computing system may increase or decrease the frequency of receiving the generated content. The audio AR computing system may alter the voice of the generated AR content based on the AR setting.

In examples, the audio AR computing system may adjust the generated AR content based on the adjustment indication indicating audio information for a critical surgical step. The audio AR computing system may silence the audio data from the sensing system. For example, the audio AR computing system may adjust the AR content by blocking (e.g., temporarily blocking) the audio data from the sensing system and allowing audible information associated with the critical surgical step. The audio AR computing system may increase the volume of the audio information for the critical surgical step. The user of the audio AR computing system may receive the audible information for the critical surgical step and may listen to and/or focus on the audible information associated with the critical surgical step.

In examples, the audio AR computing system may adjust the generated AR content based on the audio information for the critical surgical step. The audio information for the critical surgical step may be or may include an increase frequency indication or a decrease frequency indication (e.g., such as an AR setting). The audio AR computing system may increase the frequency of the audible information for the critical surgical step based on the increase frequency indication. The audio AR computing system may send an increase frequency request to other computing system (e.g., a surgical computing system and/or a central computing system) and may request increase a frequency of sending the audible information. The audio AR computing system may decrease the frequency of the audible information for the critical surgical step based on the decrease frequency indication. The audio AR computing system may send a decrease frequency request to other computing system (e.g., a surgical computing system, a central computing system, and/or a surgical hub) and may request decrease a frequency of sending the audible information for the critical surgical step.

In examples, the audio AR computing system may adjust the generated AR content based on a surgical task indication included (e.g., comprised) in the adjustment indication. The surgical task indication may indicate a surgical task that is being performed or that is to be performed. The audio AR computing system may identify an audio AR setting associated with audible information associated the surgical task. The audio AR computing system may adjust the AR content in accordance with the identified audio AR setting. For example, as described herein, the audio AR computing system may adjust the AR content by increasing or decreasing the volume of the audible information associated with the surgical task and/or increasing or decreasing the frequency of receiving the audible information.

In examples, the audio AR computing system may adjust the generated AR content based on a surgical task indication included (e.g., comprised) in the adjustment indication. The surgical task indication may indicate a surgical task that is being performed or that is to be performed. The audio AR computing system may identify a relevance of the audio data from the sensing system to the surgical task indicated in the surgical task indication. For example, the audio AR computing system may receive one or more measurement data from the one or more sensing systems in the OR. The audio AR computing system may identify (e.g., determine) the relevance of the audio data associated with the measurement data from the sensing systems. The audio AR computing system may determine whether to block the audio data from the sensing system. For example, the audio AR computing system may determine whether to block the audio data from the one or more sensing systems based on the identified relevance to the surgical task indicated in the surgical task indication. If the audio AR computing system determines that one or more audio data from the one or more sensing system are irrelevant to the surgical task indicated in the surgical task indication, the audio AR computing system may block the one or more audio data. If the audio AR computing system determines that one or more audio data from the one or more sensing system are relevant to the surgical task indicated in the surgical task indication, the audio AR computing system may allow the one or more audio data and play the audio data.

In examples, the audio AR computing system may adjust the generated AR content by blocking an ambient noise of the OR. For example, the audio AR computing system may receive audio data that may include ambient noise of the OR (e.g., HCPs talking to one another, sounds of surgical instruments, and/or the like). The audio AR computing system may cancel and/or block the ambient noise.

In examples, the audio AR computing system may receive audible data with ambient noise blocked. The audio AR computing system may generate AR content without the ambient noise.

In examples, the audio AR computing system may receive an ambient noise level indication. The ambient noise level indication may indicate an ambient noise level of the OR. In examples, the audio AR computing system may detect the ambient noise level of the OR. If the audio AR computing system determines that the ambient noise level of the OR is below a threshold ambient noise level, the audio AR computing system may be aware and/or determine that a critical step and/or task is to be performed. The audio AR computing system may send a critical task indication to other computing system (e.g., a surgical computing system). The critical task indication may indicate a critical surgical task is to be performed. The audio AR computing system may adjust the AR content and may receive audible information for a critical surgical task.

In examples, the audio AR computing system may request a user input for adjusting the AR content. For example, the audio AR computing system may request a user input prior to adjusting the AR content. The audio AR computing system may send a user input request to other computing system (e.g., a surgical computing system). The user input request may request a user input before adjusting the generated AR content. The audio AR computing system may wait for a response for the user input for a preconfigured time. If the audio AR computing system does not receive a response for the user input, the audio AR computing system may send a reminder user input request and/or send a user input request to other HCPs in the OR.

In examples, the audio AR computing system may receive one or more audio data. For example, the audio AR computing system may receive audible information from a sensing system and receive another audible information from another sensing system. The audio AR computing system may obtain the adjustment indication. The adjustment indication may be or may include a user preference setting associated with a surgical operation. Based on the user preference setting, the audio AR computing system may adjust the AR content by selecting and/or receiving the audio information from the sensing system (e.g., the first audible information from the first sensing system). The audio AR computing system may block the other audio information from the other sensing system (e.g., the second audible information from the second sensing system).

The user preference setting may indicate a preferred audio data if the AR computing system receives audible data from multiple sensing systems. The AR computing system may adjust the AR content by increasing a volume of the selected and/or preferred audio data. The audio AR computing system may reduce the volume of the unselected audio data. The audio AR computing system may cancel and/or block the unselected audio data. The audio AR computing system may request an increase frequency of receiving the selected and/or preferred audio data. The audio AR computing system may send a decrease frequency of receiving the unselected audio data.

In examples, the audio AR computing system may adjust the AR content based on the adjustment indication indicating a surgical step indication. The surgical step indication may indicate a current surgical step associated with the surgical operation. The audio AR computing system may receive audio data associated with an HCP role in the OR. The audio AR computing system may receive other audio data associated with other HCP role in the OR. The audio AR computing system may adjust the AR content to allow the audio data (e.g., the first audio data) associated with the HCP role (e.g., first HCP role) and may block the other audio data (e.g., the second audio data) associated with the other HCP role (e.g., second HCP role)

### BRIEF DESCRIPTION OF THE DRAWINGS

FIG. 1A is a block diagram of a computer-implemented patient and surgeon monitoring system.

FIG. 1B is a block diagram of an example relationship among sensing systems, biomarkers, and physiologic systems.

FIG. 2A shows an example of a surgeon monitoring system in a surgical operating room.

FIG. 2B shows an example of a patient monitoring system (e.g., a controlled patient monitoring system).

FIG. 2C shows an example of a patient monitoring system (e.g., an uncontrolled patient monitoring system).

FIG. 3 illustrates an example surgical hub paired with various systems.

FIG. 4 illustrates a surgical data network having a set of communication surgical hubs configured to connect with a set of sensing systems, an environmental sensing system, a set of devices, etc.

FIG. 5 illustrates an example computer-implemented interactive surgical system that may be part of a surgeon monitoring system.

FIG. 6A illustrates a surgical hub comprising a plurality of modules coupled to a modular control tower.

FIG. 6B illustrates an example of a controlled patient monitoring system.

FIG. 6C illustrates an example of an uncontrolled patient monitoring system.

FIG. 7A illustrates a logic diagram of a control system of a surgical instrument or a tool.

FIG. 7B shows an exemplary sensing system with a sensor unit and a data processing and communication unit.

FIG. 7C shows an exemplary sensing system with a sensor unit and a data processing and communication unit.

FIG. 7D shows an exemplary sensing system with a sensor unit and a data processing and communication unit.

FIG. 8 illustrates an exemplary timeline of an illustrative surgical procedure indicating adjusting operational parameters of a surgical device based on a surgeon biomarker level.

FIG. 9 is a block diagram of the computer-implemented interactive surgeon/patient monitoring system.

FIG. 10 shows an example surgical system that includes a handle having a controller and a motor, an adapter releasably coupled to the handle, and a loading unit releasably coupled to the adapter.

FIGS. 11A-11D illustrate examples of sensing systems that may be used for monitoring surgeon biomarkers or patient biomarkers.

FIG. 12 is a block diagram of a patient monitoring system or a surgeon monitoring system.

FIG. 13 illustrates an example flow for an audio augmented reality (AR) computing system adjusting the AR content.

### DETAILED DESCRIPTION

Applicant of the present application owns the following U.S. patent applications, filed contemporaneously, each of which is herein incorporated by reference in its entirety:
U.S. patent application Ser. No. 16/209,416, entitled “METHOD OF HUB COMMUNICATION, PROCESSING, DISPLAY, AND CLOUD ANALYTICS,” filed Dec. 4, 2018;
U.S. patent application Ser. No. 15/940,671 (Attorney Docket No. END8502USNP), entitled “SURGICAL HUB SPATIAL AWARENESS TO DETERMINE DEVICES IN OPERATING THEATER,” filed Mar. 29, 2018;
U.S. patent application Ser. No. 16/182,269 (Attorney Docket No.: END9018USNP3) entitled “IMAGE CAPTURING OF THE AREAS OUTSIDE THE ABDOMEN TO IMPROVE PLACEMENT AND CONTROL OF A SURGICAL DEVICE IN USE,” filed Nov. 6, 2018;
U.S. patent application Ser. No. 16/729,747 (Attorney Docket No.: END9217USNP1) entitled “DYNAMIC SURGICAL VISUALIZATION SYSTEMS,” filed Dec. 31, 2019;
U.S. patent application Ser. No. 16/729,778 (Attorney Docket: END9219USNP1) entitled “SYSTEM AND METHOD FOR DETERMINING, ADJUSTING, AND MANAGING RESECTION MARGIN ABOUT A SUBJECT TISSUE,” filed Dec. 31, 2019;
U.S. patent application Ser. No. 16/729,807 (Attorney Docket: END9228USNP1) entitled METHOD OF USING IMAGING DEVICES IN SURGERY, filed Dec. 31, 2019;
U.S. patent application Ser. No. 15/940,654 (Attorney Docket No. END8501USNP), entitled SURGICAL HUB SITUATIONAL AWARENESS, filed Mar. 29, 2018;
U.S. patent application Ser. No. 15/940,671 (Attorney Docket No. END8502USNP), titled SURGICAL HUB SPATIAL AWARENESS TO DETERMINE DEVICES IN OPERATING THEATER, which was filed on Mar. 29, 2018;
U.S. patent application Ser. No. 15/940,704 (Attorney Docket No. END8504USNP), titled USE OF LASER LIGHT AND RED-GREEN-BLUE COLORATION TO DETERMINE PROPERTIES OF BACK SCATTERED LIGHT, which was filed on Mar. 29, 2018;
U.S. patent application Ser. No. 16/182,290 (Attorney Docket No. END9018USNP5), entitled “SURGICAL NETWORK RECOMMENDATIONS FROM REAL TIME ANALYSIS OF PROCEDURE VARIABLES AGAINST A BASELINE HIGHLIGHTING DIFFERENCES FROM THE OPTIMAL SOLUTION,” filed Nov. 6, 2018;
U.S. Pat. No. 9,011,427, entitled SURGICAL INSTRUMENT WITH SAFETY GLASSES, issued on Apr. 21, 2015;
U.S. Pat. No. 9,123,155, titled APPARATUS AND METHOD FOR USING AUGMENTED REALITY VISION SYSTEM IN SURGICAL PROCEDURES, which issued on Sep. 1, 2015;
U.S. patent application Ser. No. 16/209,478 (Attorney Docket No. END9015USNP1), titled METHOD FOR SITUATIONAL AWARENESS FOR SURGICAL NETWORK OR SURGICAL NETWORK CONNECTED DEVICE CAPABLE OF ADJUSTING FUNCTION BASED ON A SENSED SITUATION OR USAGE, filed Dec. 4, 2018; and
U.S. patent application Ser. No. 16/182,246 (Attorney Docket No. END9016USNP1), titled ADJUSTMENTS BASED ON AIRBORNE PARTICLE PROPERTIES, filed Nov. 6, 2018.

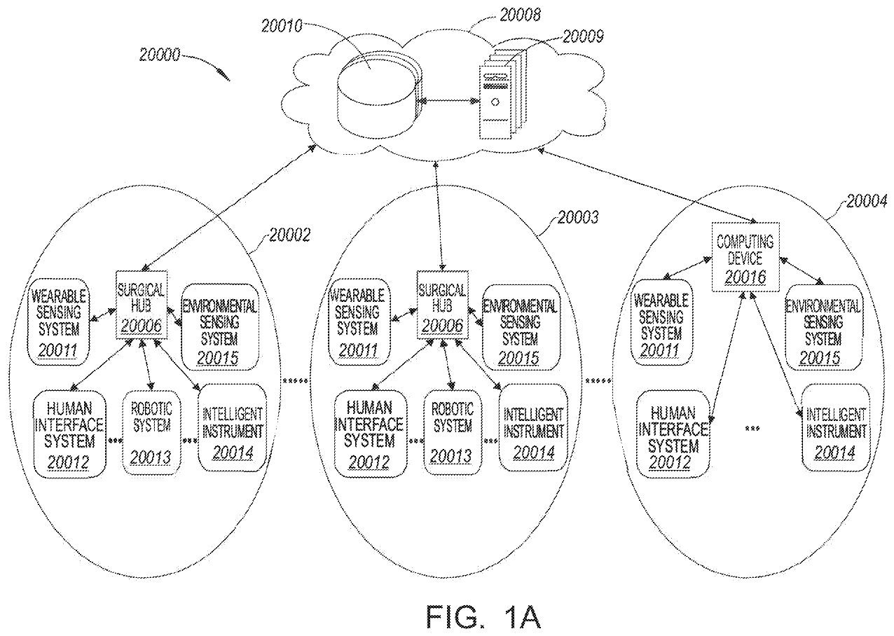

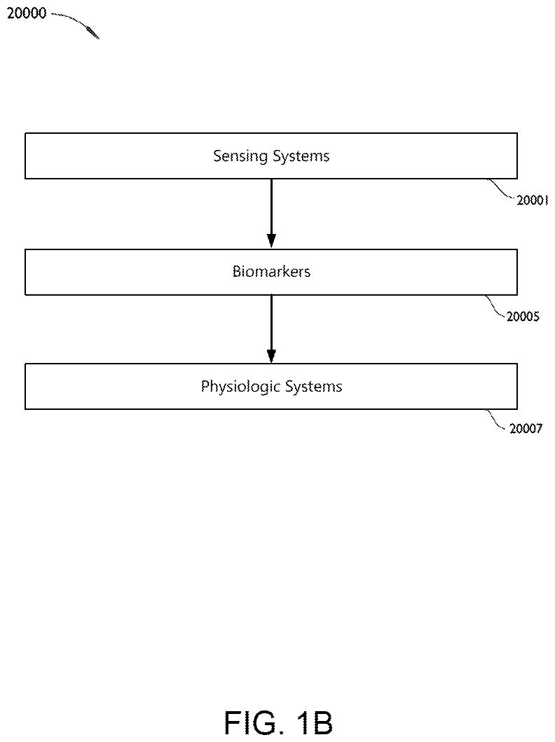

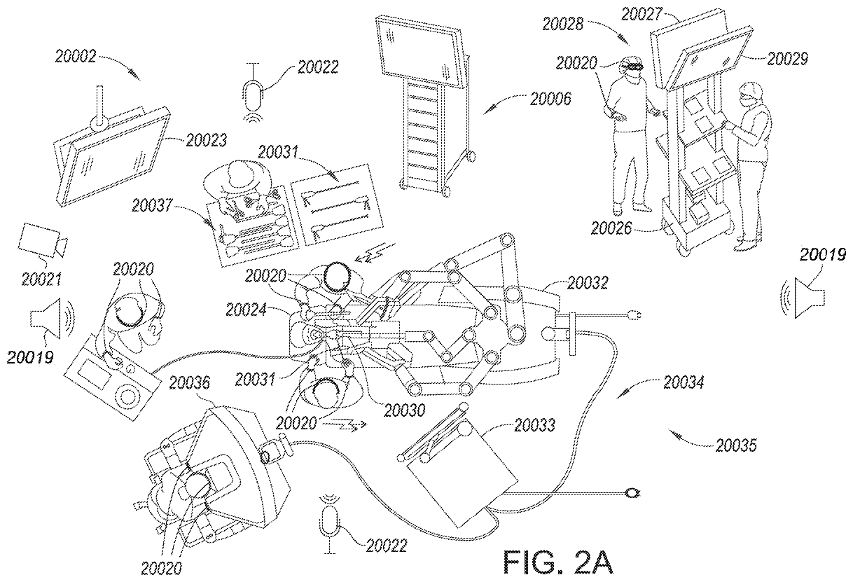

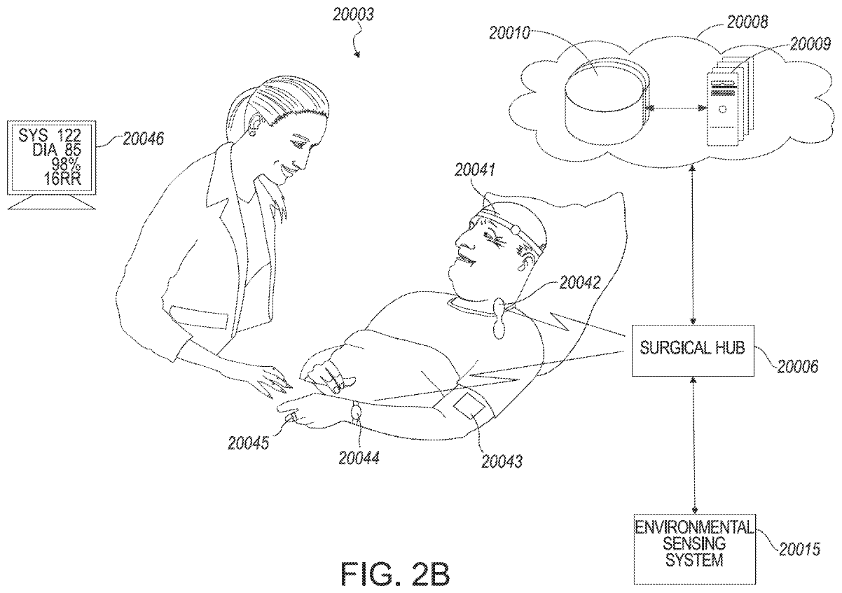

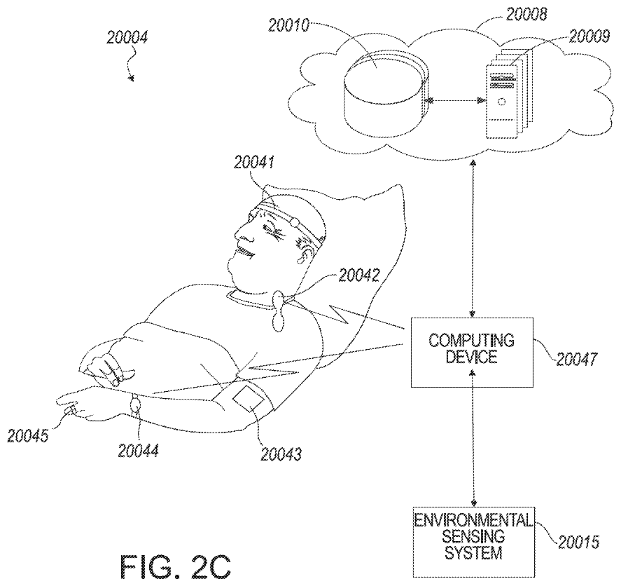

FIG. 1A is a block diagram of a computer-implemented patient and surgeon monitoring system 20000. The patient and surgeon monitoring system 20000 may include one or more surgeon monitoring systems 20002 and a one or more patient monitoring systems (e.g., one or more controlled patient monitoring systems 20003 and one or more uncontrolled patient monitoring systems 20004). Each surgeon monitoring system 20002 may include a computer-implemented interactive surgical system. Each surgeon monitoring system 20002 may include at least one of the following: a surgical hub 20006 in communication with a cloud computing system 20008, for example, as described in FIG. 2A. Each of the patient monitoring systems may include at least one of the following: a surgical hub 20006 or a computing device 20016 in communication with a could computing system 20008, for example, as further described in FIG. 2B and FIG. 2C. The cloud computing system 20008 may include at least one remote cloud server 20009 and at least one remote cloud storage unit 20010. Each of the surgeon monitoring systems 20002, the controlled patient monitoring systems 20003, or the uncontrolled patient monitoring systems 20004 may include a wearable sensing system 20011, an environmental sensing system 20015, a robotic system 20013, one or more intelligent instruments 20014, human interface system 20012, etc. The human interface system is also referred herein as the human interface device. The wearable sensing system 20011 may include one or more surgeon sensing systems, and/or one or more patient sensing systems. The environmental sensing system 20015 may include one or more devices, for example, used for measuring one or more environmental attributes, for example, as further described in FIG. 2A. The robotic system 20013 (same as 20034 in FIG. 2A) may include a plurality of devices used for performing a surgical procedure, for example, as further described in FIG. 2A.

A surgical hub 20006 may have cooperative interactions with one of more means of displaying the image from the laparoscopic scope and information from one or more other smart devices and one or more sensing systems 20011. The surgical hub 20006 may interact with one or more sensing systems 20011, one or more smart devices, and multiple displays. The surgical hub 20006 may be configured to gather measurement data from the one or more sensing systems 20011 and send notifications or control messages to the one or more sensing systems 20011. The surgical hub 20006 may send and/or receive information including notification information to and/or from the human interface system 20012. The human interface system 20012 may include one or more human interface devices (HIDs). The surgical hub 20006 may send and/or receive notification information or control information to audio, display and/or control information to various devices that are in communication with the surgical hub.

FIG. 1B is a block diagram of an example relationship among sensing systems 20001, biomarkers 20005, and physiologic systems 20007. The relationship may be employed in the computer-implemented patient and surgeon monitoring system 20000 and in the systems, devices, and methods disclosed herein. For example, the sensing systems 20001 may include the wearable sensing system 20011 (which may include one or more surgeon sensing systems and one or more patient sensing systems) and the environmental sensing system 20015 as discussed in FIG. 1A. The one or more sensing systems 20001 may measure data relating to various biomarkers 20005. The one or more sensing systems 20001 may measure the biomarkers 20005 using one or more sensors, for example, photosensors (e.g., photodiodes, photoresistors), mechanical sensors (e.g., motion sensors), acoustic sensors, electrical sensors, electrochemical sensors, thermoelectric sensors, infrared sensors, etc. The one or more sensors may measure the biomarkers 20005 as described herein using one of more of the following sensing technologies: photoplethysmography, electrocardiography, electroencephalography, colorimetry, impedimentary, potentiometry, amperometry, etc.

The biomarkers 20005 measured by the one or more sensing systems 20001 may include, but are not limited to, sleep, core body temperature, maximal oxygen consumption, physical activity, alcohol consumption, respiration rate, oxygen saturation, blood pressure, blood sugar, heart rate variability, blood potential of hydrogen, hydration state, heart rate, skin conductance, peripheral temperature, tissue perfusion pressure, coughing and sneezing, gastrointestinal motility, gastrointestinal tract imaging, respiratory tract bacteria, edema, mental aspects, sweat, circulating tumor cells, autonomic tone, circadian rhythm, and/or menstrual cycle.

The biomarkers 20005 may relate to physiologic systems 20007, which may include, but are not limited to, behavior and psychology, cardiovascular system, renal system, skin system, nervous system, gastrointestinal system, respiratory system, endocrine system, immune system, tumor, musculoskeletal system, and/or reproductive system. Information from the biomarkers may be determined and/or used by the computer-implemented patient and surgeon monitoring system 20000, for example. The information from the biomarkers may be determined and/or used by the computer-implemented patient and surgeon monitoring system 20000 to improve said systems and/or to improve patient outcomes, for example.

FIG. 2A shows an example of a surgeon monitoring system 20002 in a surgical operating room. As illustrated in FIG. 2A, a patient is being operated on by one or more health care professionals (HCPs). The HCPs are being monitored by one or more surgeon sensing systems 20020 worn by the HCPs. The HCPs and the environment surrounding the HCPs may also be monitored by one or more environmental sensing systems including, for example, a set of cameras 20021, a set of microphones 20022, and other sensors, etc. that may be deployed in the operating room. The surgeon sensing systems 20020 and the environmental sensing systems may be in communication with a surgical hub 20006, which in turn may be in communication with one or more cloud servers 20009 of the cloud computing system 20008, as shown in FIG. 1A. The environmental sensing systems may be used for measuring one or more environmental attributes, for example, HCP position in the surgical theater, HCP movements, ambient noise in the surgical theater, temperature/humidity in the surgical theater, etc.

As illustrated in FIG. 2A, a primary display 20023 and one or more audio output devices (e.g., speakers 20019) are positioned in the sterile field to be visible to an operator at the operating table 20024. In addition, a visualization/notification tower 20026 is positioned outside the sterile field. The visualization/notification tower 20026 may include a first non-sterile human interactive device (HID) 20027 and a second non-sterile HID 20029, which may face away from each other. The HID may be a display or a display with a touchscreen allowing a human to interface directly with the HID. A human interface system, guided by the surgical hub 20006, may be configured to utilize the HIDs 20027, 20029, and 20023 to coordinate information flow to operators inside and outside the sterile field. In an example, the surgical hub 20006 may cause an HID (e.g., the primary HID 20023) to display a notification and/or information about the patient and/or a surgical procedure step. In an example, the surgical hub 20006 may prompt for and/or receive input from personnel in the sterile field or in the non-sterile area. In an example, the surgical hub 20006 may cause an HID to display a snapshot of a surgical site, as recorded by an imaging device 20030, on a non-sterile HID 20027 or 20029, while maintaining a live feed of the surgical site on the primary HID 20023. The snapshot on the non-sterile display 20027 or 20029 can permit a non-sterile operator to perform a diagnostic step relevant to the surgical procedure, for example.

In one aspect, the surgical hub 20006 may be configured to route a diagnostic input or feedback entered by a non-sterile operator at the visualization tower 20026 to the primary display 20023 within the sterile field, where it can be viewed by a sterile operator at the operating table. In one example, the input can be in the form of a modification to the snapshot displayed on the non-sterile display 20027 or 20029, which can be routed to the primary display 20023 by the surgical hub 20006.

Referring to FIG. 2A, a surgical instrument 20031 is being used in the surgical procedure as part of the surgeon monitoring system 20002. The hub 20006 may be configured to coordinate information flow to a display of the surgical instrument 20031. For example, in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. A diagnostic input or feedback entered by a non-sterile operator at the visualization tower 20026 can be routed by the hub 20006 to the surgical instrument display within the sterile field, where it can be viewed by the operator of the surgical instrument 20031. Example surgical instruments that are suitable for use with the surgical system 20002 are described under the heading “Surgical Instrument Hardware” and in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety, for example.

FIG. 2A illustrates an example of a surgical system 20002 being used to perform a surgical procedure on a patient who is lying down on an operating table 20024 in a surgical operating room 20035. A robotic system 20034 may be used in the surgical procedure as a part of the surgical system 20002. The robotic system 20034 may include a surgeon's console 20036, a patient side cart 20032 (surgical robot), and a surgical robotic hub 20033. The patient side cart 20032 can manipulate at least one removably coupled surgical tool 20037 through a minimally invasive incision in the body of the patient while the surgeon views the surgical site through the surgeon's console 20036. An image of the surgical site can be obtained by a medical imaging device 20030, which can be manipulated by the patient side cart 20032 to orient the imaging device 20030. The robotic hub 20033 can be used to process the images of the surgical site for subsequent display to the surgeon through the surgeon's console 20036.

Other types of robotic systems can be readily adapted for use with the surgical system 20002. Various examples of robotic systems and surgical tools that are suitable for use with the present disclosure are described in U.S. Patent Application Publication No. US 2019-0201137 A1 (U.S. patent application Ser. No. 16/209,407), titled METHOD OF ROBOTIC HUB COMMUNICATION, DETECTION, AND CONTROL, filed Dec. 4, 2018, the disclosure of which is herein incorporated by reference in its entirety.

Various examples of cloud-based analytics that are performed by the cloud computing system 20008, and are suitable for use with the present disclosure, are described in U.S. Patent Application Publication No. US 2019-0206569 A1 (U.S. patent application Ser. No. 16/209,403), titled METHOD OF CLOUD BASED DATA ANALYTICS FOR USE WITH THE HUB, filed Dec. 4, 2018, the disclosure of which is herein incorporated by reference in its entirety.

In various aspects, the imaging device 20030 may include at least one image sensor and one or more optical components. Suitable image sensors may include, but are not limited to, Charge-Coupled Device (CCD) sensors and Complementary Metal-Oxide Semiconductor (CMOS) sensors.

The optical components of the imaging device 20030 may include one or more illumination sources and/or one or more lenses. The one or more illumination sources may be directed to illuminate portions of the surgical field. The one or more image sensors may receive light reflected or refracted from the surgical field, including light reflected or refracted from tissue and/or surgical instruments.

The one or more illumination sources may be configured to radiate electromagnetic energy in the visible spectrum as well as the invisible spectrum. The visible spectrum, sometimes referred to as the optical spectrum or luminous spectrum, is that portion of the electromagnetic spectrum that is visible to (i.e., can be detected by) the human eye and may be referred to as visible light or simply light. A typical human eye will respond to wavelengths in air that range from about 380 nm to about 750 nm.

The invisible spectrum (e.g., the non-luminous spectrum) is that portion of the electromagnetic spectrum that lies below and above the visible spectrum (i.e., wavelengths below about 380 nm and above about 750 nm). The invisible spectrum is not detectable by the human eye. Wavelengths greater than about 750 nm are longer than the red visible spectrum, and they become invisible infrared (IR), microwave, and radio electromagnetic radiation. Wavelengths less than about 380 nm are shorter than the violet spectrum, and they become invisible ultraviolet, x-ray, and gamma ray electromagnetic radiation.

In various aspects, the imaging device 20030 is configured for use in a minimally invasive procedure. Examples of imaging devices suitable for use with the present disclosure include, but are not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope.

The imaging device may employ multi-spectrum monitoring to discriminate topography and underlying structures. A multi-spectral image is one that captures image data within specific wavelength ranges across the electromagnetic spectrum. The wavelengths may be separated by filters or by the use of instruments that are sensitive to particular wavelengths, including light from frequencies beyond the visible light range, e.g., IR and ultraviolet. Spectral imaging can allow extraction of additional information that the human eye fails to capture with its receptors for red, green, and blue. The use of multi-spectral imaging is described in greater detail under the heading “Advanced Imaging Acquisition Module” in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. Multi-spectrum monitoring can be a useful tool in relocating a surgical field after a surgical task is completed to perform one or more of the previously described tests on the treated tissue. It is axiomatic that strict sterilization of the operating room and surgical equipment is required during any surgery. The strict hygiene and sterilization conditions required in a “surgical theater,” i.e., an operating or treatment room, necessitate the highest possible sterility of all medical devices and equipment. Part of that sterilization process is the need to sterilize anything that comes in contact with the patient or penetrates the sterile field, including the imaging device 20030 and its attachments and components. It will be appreciated that the sterile field may be considered a specified area, such as within a tray or on a sterile towel, that is considered free of microorganisms, or the sterile field may be considered an area, immediately around a patient, who has been prepared for a surgical procedure. The sterile field may include the scrubbed team members, who are properly attired, and all furniture and fixtures in the area.

Wearable sensing system 20011 illustrated in FIG. 1A may include one or more sensing systems, for example, surgeon sensing systems 20020 as shown in FIG. 2A. The surgeon sensing systems 20020 may include sensing systems to monitor and detect a set of physical states and/or a set of physiological states of a healthcare provider (HCP). An HCP may be a surgeon or one or more healthcare personnel assisting the surgeon or other healthcare service providers in general. In an example, a sensing system 20020 may measure a set of biomarkers to monitor the heart rate of an HCP. In another example, a sensing system 20020 worn on a surgeon's wrist (e.g., a watch or a wristband) may use an accelerometer to detect hand motion and/or shakes and determine the magnitude and frequency of tremors. The sensing system 20020 may send the measurement data associated with the set of biomarkers and the data associated with a physical state of the surgeon to the surgical hub 20006 for further processing. One or more environmental sensing devices may send environmental information to the surgical hub 20006. For example, the environmental sensing devices may include a camera 20021 for detecting hand/body position of an HCP. The environmental sensing devices may include microphones 20022 for measuring the ambient noise in the surgical theater. Other environmental sensing devices may include devices, for example, a thermometer to measure temperature and a hygrometer to measure humidity of the surroundings in the surgical theater, etc. The surgical hub 20006, alone or in communication with the cloud computing system, may use the surgeon biomarker measurement data and/or environmental sensing information to modify the control algorithms of hand-held instruments or the averaging delay of a robotic interface, for example, to minimize tremors. In an example, the surgeon sensing systems 20020 may measure one or more surgeon biomarkers associated with an HCP and send the measurement data associated with the surgeon biomarkers to the surgical hub 20006. The surgeon sensing systems 20020 may use one or more of the following RF protocols for communicating with the surgical hub 20006: Bluetooth, Bluetooth Low-Energy (BLE), Bluetooth Smart, Zigbee, Z-wave, IPv6 Low-power wireless Personal Area Network (6LoWPAN), Wi-Fi. The surgeon biomarkers may include one or more of the following: stress, heart rate, etc. The environmental measurements from the surgical theater may include ambient noise level associated with the surgeon or the patient, surgeon and/or staff movements, surgeon and/or staff attention level, etc.

The surgical hub 20006 may use the surgeon biomarker measurement data associated with an HCP to adaptively control one or more surgical instruments 20031. For example, the surgical hub 20006 may send a control program to a surgical instrument 20031 to control its actuators to limit or compensate for fatigue and use of fine motor skills. The surgical hub 20006 may send the control program based on situational awareness and/or the context on importance or criticality of a task. The control program may instruct the instrument to alter operation to provide more control when control is needed.

FIG. 2B shows an example of a patient monitoring system 20003 (e.g., a controlled patient monitoring system). As illustrated in FIG. 2B, a patient in a controlled environment (e.g., in a hospital recovery room) may be monitored by a plurality of sensing systems (e.g., patient sensing systems 20041). A patient sensing system 20041 (e.g., a head band) may be used to measure an electroencephalogram (EEG) to measure electrical activity of the brain of a patient. A patient sensing system 20042 may be used to measure various biomarkers of the patient including, for example, heart rate, VO2 level, etc. A patient sensing system 20043 (e.g., flexible patch attached to the patient's skin) may be used to measure sweat lactate and/or potassium levels by analyzing small amounts of sweat that is captured from the surface of the skin using microfluidic channels. A patient sensing system 20044 (e.g., a wristband or a watch) may be used to measure blood pressure, heart rate, heart rate variability, VO2 levels, etc. using various techniques, as described herein. A patient sensing system 20045 (e.g., a ring on finger) may be used to measure peripheral temperature, heart rate, heart rate variability, VO2 levels, etc. using various techniques, as described herein. The patient sensing systems 20041-20045 may use a radio frequency (RF) link to be in communication with the surgical hub 20006. The patient sensing systems 20041-20045 may use one or more of the following RF protocols for communication with the surgical hub 20006: Bluetooth, Bluetooth Low-Energy (BLE), Bluetooth Smart, Zigbee, Z-wave, IPv6 Low-power wireless Personal Area Network (6LoWPAN), Thread, Wi-Fi, etc.

The sensing systems 20041-20045 may be in communication with a surgical hub 20006, which in turn may be in communication with a remote server 20009 of the remote cloud computing system 20008. The surgical hub 20006 is also in communication with an HID 20046. The HID 20046 may display measured data associated with one or more patient biomarkers. For example, the HID 20046 may display blood pressure, Oxygen saturation level, respiratory rate, etc. The HID 20046 may display notifications for the patient or an HCP providing information about the patient, for example, information about a recovery milestone or a complication. In an example, the information about a recovery milestone or a complication may be associated with a surgical procedure the patient may have undergone. In an example, the HID 20046 may display instructions for the patient to perform an activity. For example, the HID 20046 may display inhaling and exhaling instructions. In an example the HID 20046 may be part of a sensing system.

As illustrated in FIG. 2B, the patient and the environment surrounding the patient may be monitored by one or more environmental sensing systems 20015 including, for example, a microphone (e.g., for detecting ambient noise associated with or around a patient), a temperature/humidity sensor, a camera for detecting breathing patterns of the patient, etc. The environmental sensing systems 20015 may be in communication with the surgical hub 20006, which in turn is in communication with a remote server 20009 of the remote cloud computing system 20008.

In an example, a patient sensing system 20044 may receive a notification information from the surgical hub 20006 for displaying on a display unit or an HID of the patient sensing system 20044. The notification information may include a notification about a recovery milestone or a notification about a complication, for example, in case of post-surgical recovery. In an example, the notification information may include an actionable severity level associated with the notification. The patient sensing system 20044 may display the notification and the actionable severity level to the patient. The patient sensing system may alert the patient using a haptic feedback. The visual notification and/or the haptic notification may be accompanied by an audible notification prompting the patient to pay attention to the visual notification provided on the display unit of the sensing system.

FIG. 2C shows an example of a patient monitoring system (e.g., an uncontrolled patient monitoring system 20004). As illustrated in FIG. 2C, a patient in an uncontrolled environment (e.g., a patient's residence) is being monitored by a plurality of patient sensing systems 20041-20045. The patient sensing systems 20041-20045 may measure and/or monitor measurement data associated with one or more patient biomarkers. For example, a patient sensing system 20041, a head band, may be used to measure an electroencephalogram (EEG). Other patient sensing systems 20042, 20043, 20044, and 20045 are examples where various patient biomarkers are monitored, measured, and/or reported, as described in FIG. 2B. One or more of the patient sensing systems 20041-20045 may be send the measured data associated with the patient biomarkers being monitored to the computing device 20047, which in turn may be in communication with a remote server 20009 of the remote cloud computing system 20008. The patient sensing systems 20041-20045 may use a radio frequency (RF) link to be in communication with a computing device 20047 (e.g., a smart phone, a tablet, etc.). The patient sensing systems 20041-20045 may use one or more of the following RF protocols for communication with the computing device 20047: Bluetooth, Bluetooth Low-Energy (BLE), Bluetooth Smart, Zigbee, Z-wave, IPv6 Low-power wireless Personal Area Network (6LoWPAN), Thread, Wi-Fi, etc. In an example, the patient sensing systems 20041-20045 may be connected to the computing device 20047 via a wireless router, a wireless hub, or a wireless bridge.

The computing device 20047 may be in communication with a remote server 20009 that is part of a cloud computing system 20008. In an example, the computing device 20047 may be in communication with a remote server 20009 via an internet service provider's cable/FIOS networking node. In an example, a patient sensing system may be in direct communication with a remote server 20009. The computing device 20047 or the sensing system may communicate with the remote servers 20009 via a cellular transmission/reception point (TRP) or a base station using one or more of the following cellular protocols: GSM/GPRS/EDGE (2G), UMTS/HSPA (3G), long term evolution (LTE) or 4G, LTE-Advanced (LTE-A), new radio (NR) or 5G.

In an example, a computing device 20047 may display information associated with a patient biomarker. For example, a computing device 20047 may display blood pressure, Oxygen saturation level, respiratory rate, etc. A computing device 20047 may display notifications for the patient or an HCP providing information about the patient, for example, information about a recovery milestone or a complication.

In an example, the computing device 20047 and/or the patient sensing system 20044 may receive a notification information from the surgical hub 20006 for displaying on a display unit of the computing device 20047 and/or the patient sensing system 20044. The notification information may include a notification about a recovery milestone or a notification about a complication, for example, in case of post-surgical recovery. The notification information may also include an actionable severity level associated with the notification. The computing device 20047 and/or the sensing system 20044 may display the notification and the actionable severity level to the patient. The patient sensing system may also alert the patient using a haptic feedback. The visual notification and/or the haptic notification may be accompanied by an audible notification prompting the patient to pay attention to the visual notification provided on the display unit of the sensing system.

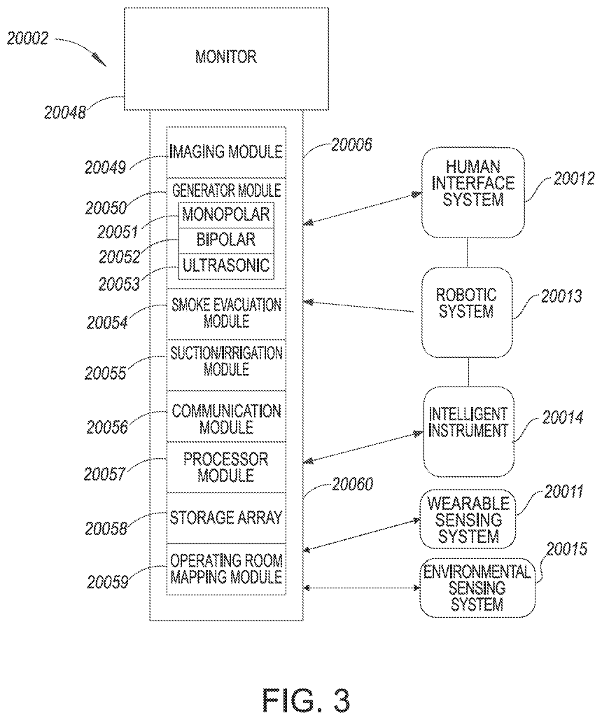

FIG. 3 shows an example surgeon monitoring system 20002 with a surgical hub 20006 paired with a wearable sensing system 20011, an environmental sensing system 20015, a human interface system 20012, a robotic system 20013, and an intelligent instrument 20014. The hub 20006 includes a display 20048, an imaging module 20049, a generator module 20050, a communication module 20056, a processor module 20057, a storage array 20058, and an operating-room mapping module 20059. In certain aspects, as illustrated in FIG. 3, the hub 20006 further includes a smoke evacuation module 20054 and/or a suction/irrigation module 20055. During a surgical procedure, energy application to tissue, for sealing and/or cutting, is generally associated with smoke evacuation, suction of excess fluid, and/or irrigation of the tissue. Fluid, power, and/or data lines from different sources are often entangled during the surgical procedure. Valuable time can be lost addressing this issue during a surgical procedure. Detangling the lines may necessitate disconnecting the lines from their respective modules, which may require resetting the modules. The hub modular enclosure 20060 offers a unified environment for managing the power, data, and fluid lines, which reduces the frequency of entanglement between such lines. Aspects of the present disclosure present a surgical hub 20006 for use in a surgical procedure that involves energy application to tissue at a surgical site. The surgical hub 20006 includes a hub enclosure 20060 and a combo generator module slidably receivable in a docking station of the hub enclosure 20060. The docking station includes data and power contacts. The combo generator module includes two or more of an ultrasonic energy generator component, a bipolar RF energy generator component, and a monopolar RF energy generator component that are housed in a single unit. In one aspect, the combo generator module also includes a smoke evacuation component, at least one energy delivery cable for connecting the combo generator module to a surgical instrument, at least one smoke evacuation component configured to evacuate smoke, fluid, and/or particulates generated by the application of therapeutic energy to the tissue, and a fluid line extending from the remote surgical site to the smoke evacuation component. In one aspect, the fluid line may be a first fluid line, and a second fluid line may extend from the remote surgical site to a suction and irrigation module 20055 slidably received in the hub enclosure 20060. In one aspect, the hub enclosure 20060 may include a fluid interface. Certain surgical procedures may require the application of more than one energy type to the tissue. One energy type may be more beneficial for cutting the tissue, while another different energy type may be more beneficial for sealing the tissue. For example, a bipolar generator can be used to seal the tissue while an ultrasonic generator can be used to cut the sealed tissue. Aspects of the present disclosure present a solution where a hub modular enclosure 20060 is configured to accommodate different generators and facilitate an interactive communication therebetween. One of the advantages of the hub modular enclosure 20060 is enabling the quick removal and/or replacement of various modules. Aspects of the present disclosure present a modular surgical enclosure for use in a surgical procedure that involves energy application to tissue. The modular surgical enclosure includes a first energy-generator module, configured to generate a first energy for application to the tissue, and a first docking station comprising a first docking port that includes first data and power contacts, wherein the first energy-generator module is slidably movable into an electrical engagement with the power and data contacts and wherein the first energy-generator module is slidably movable out of the electrical engagement with the first power and data contacts. Further to the above, the modular surgical enclosure also includes a second energy-generator module configured to generate a second energy, different than the first energy, for application to the tissue, and a second docking station comprising a second docking port that includes second data and power contacts, wherein the second energy-generator module is slidably movable into an electrical engagement with the power and data contacts, and wherein the second energy-generator module is slidably movable out of the electrical engagement with the second power and data contacts. In addition, the modular surgical enclosure also includes a communication bus between the first docking port and the second docking port, configured to facilitate communication between the first energy-generator module and the second energy-generator module. Referring to FIG. 3, aspects of the present disclosure are presented for a hub modular enclosure 20060 that allows the modular integration of a generator module 20050, a smoke evacuation module 20054, and a suction/irrigation module 20055. The hub modular enclosure 20060 further facilitates interactive communication between the modules 20059, 20054, and 20055. The generator module 20050 can be a generator module 20050 with integrated monopolar, bipolar, and ultrasonic components supported in a single housing unit slidably insertable into the hub modular enclosure 20060. The generator module 20050 can be configured to connect to a monopolar device 20051, a bipolar device 20052, and an ultrasonic device 20053. Alternatively, the generator module 20050 may comprise a series of monopolar, bipolar, and/or ultrasonic generator modules that interact through the hub modular enclosure 20060. The hub modular enclosure 20060 can be configured to facilitate the insertion of multiple generators and interactive communication between the generators docked into the hub modular enclosure 20060 so that the generators would act as a single generator.

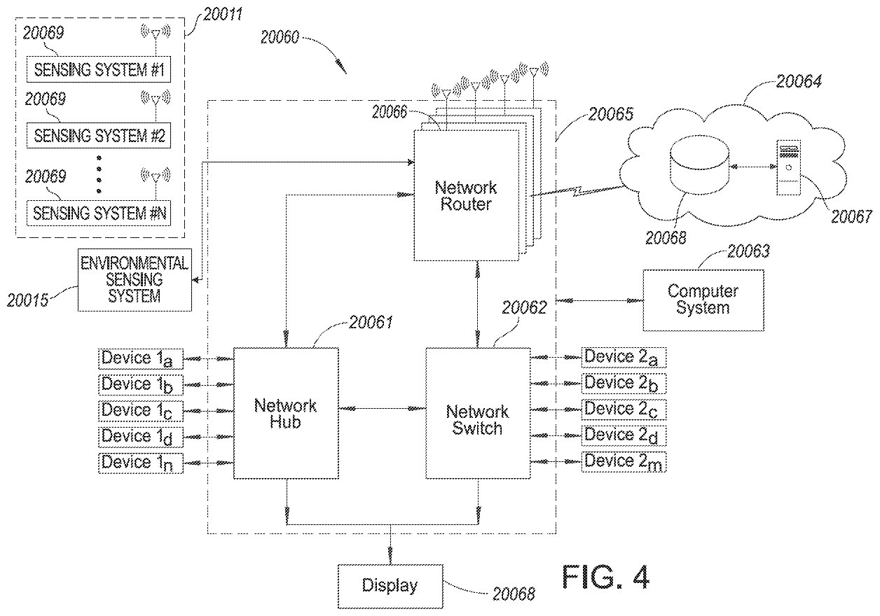

FIG. 4 illustrates a surgical data network having a set of communication hubs configured to connect a set of sensing systems, an environment sensing system, and a set of other modular devices located in one or more operating theaters of a healthcare facility, a patient recovery room, or a room in a healthcare facility specially equipped for surgical operations, to the cloud, in accordance with at least one aspect of the present disclosure.

As illustrated in FIG. 4, a surgical hub system 20060 may include a modular communication hub 20065 that is configured to connect modular devices located in a healthcare facility to a cloud-based system (e.g., a cloud computing system 20064 that may include a remote server 20067 coupled to a remote storage 20068). The modular communication hub 20065 and the devices may be connected in a room in a healthcare facility specially equipped for surgical operations. In one aspect, the modular communication hub 20065 may include a network hub 20061 and/or a network switch 20062 in communication with a network router 20066. The modular communication hub 20065 may be coupled to a local computer system 20063 to provide local computer processing and data manipulation. Surgical data network associated with the surgical hub system 20060 may be configured as passive, intelligent, or switching. A passive surgical data network serves as a conduit for the data, enabling it to go from one device (or segment) to another and to the cloud computing resources. An intelligent surgical data network includes additional features to enable the traffic passing through the surgical data network to be monitored and to configure each port in the network hub 20061 or network switch 20062. An intelligent surgical data network may be referred to as a manageable hub or switch. A switching hub reads the destination address of each packet and then forwards the packet to the correct port.

Modular devices 1a-1n located in the operating theater may be coupled to the modular communication hub 20065. The network hub 20061 and/or the network switch 20062 may be coupled to a network router 20066 to connect the devices 1a-1n to the cloud computing system 20064 or the local computer system 20063. Data associated with the devices 1a-1n may be transferred to cloud-based computers via the router for remote data processing and manipulation. Data associated with the devices 1a-1n may also be transferred to the local computer system 20063 for local data processing and manipulation. Modular devices 2a-2m located in the same operating theater also may be coupled to a network switch 20062. The network switch 20062 may be coupled to the network hub 20061 and/or the network router 20066 to connect the devices 2a-2m to the cloud 20064. Data associated with the devices 2a-2m may be transferred to the cloud computing system 20064 via the network router 20066 for data processing and manipulation. Data associated with the devices 2a-2m may also be transferred to the local computer system 20063 for local data processing and manipulation.

The wearable sensing system 20011 may include one or more sensing systems 20069. The sensing systems 20069 may include a surgeon sensing system and/or a patient sensing system. The one or more sensing systems 20069 may be in communication with the computer system 20063 of a surgical hub system 20060 or the cloud server 20067 directly via one of the network routers 20066 or via a network hub 20061 or network switching 20062 that is in communication with the network routers 20066.

The sensing systems 20069 may be coupled to the network router 20066 to connect to the sensing systems 20069 to the local computer system 20063 and/or the cloud computing system 20064. Data associated with the sensing systems 20069 may be transferred to the cloud computing system 20064 via the network router 20066 for data processing and manipulation. Data associated with the sensing systems 20069 may also be transferred to the local computer system 20063 for local data processing and manipulation.

As illustrated in FIG. 4, the surgical hub system 20060 may be expanded by interconnecting multiple network hubs 20061 and/or multiple network switches 20062 with multiple network routers 20066. The modular communication hub 20065 may be contained in a modular control tower configured to receive multiple devices 1a-1n/2a-2m. The local computer system 20063 also may be contained in a modular control tower. The modular communication hub 20065 may be connected to a display 20068 to display images obtained by some of the devices 1a-1n/2a-2m, for example during surgical procedures. In various aspects, the devices 1a-1n/2a-2m may include, for example, various modules such as an imaging module coupled to an endoscope, a generator module coupled to an energy-based surgical device, a smoke evacuation module, a suction/irrigation module, a communication module, a processor module, a storage array, a surgical device coupled to a display, and/or a non-contact sensor module, among other modular devices that may be connected to the modular communication hub 20065 of the surgical data network.

In one aspect, the surgical hub system 20060 illustrated in FIG. 4 may comprise a combination of network hub(s), network switch(es), and network router(s) connecting the devices 1a-1n/2a-2m or the sensing systems 20069 to the cloud-base system 20064. One or more of the devices 1a-1n/2a-2m or the sensing systems 20069 coupled to the network hub 20061 or network switch 20062 may collect data or measurement data in real-time and transfer the data to cloud computers for data processing and manipulation. It will be appreciated that cloud computing relies on sharing computing resources rather than having local servers or personal devices to handle software applications. The word “cloud” may be used as a metaphor for “the Internet,” although the term is not limited as such. Accordingly, the term “cloud computing” may be used herein to refer to “a type of Internet-based computing,” where different services—such as servers, storage, and applications—are delivered to the modular communication hub 20065 and/or computer system 20063 located in the surgical theater (e.g., a fixed, mobile, temporary, or field operating room or space) and to devices connected to the modular communication hub 20065 and/or computer system 20063 through the Internet. The cloud infrastructure may be maintained by a cloud service provider. In this context, the cloud service provider may be the entity that coordinates the usage and control of the devices 1a-1n/2a-2m located in one or more operating theaters. The cloud computing services can perform a large number of calculations based on the data gathered by smart surgical instruments, robots, sensing systems, and other computerized devices located in the operating theater. The hub hardware enables multiple devices, sensing systems, and/or connections to be connected to a computer that communicates with the cloud computing resources and storage.

Applying cloud computer data processing techniques on the data collected by the devices 1a-1n/2a-2m, the surgical data network can provide improved surgical outcomes, reduced costs, and improved patient satisfaction. At least some of the devices 1a-1n/2a-2m may be employed to view tissue states to assess leaks or perfusion of sealed tissue after a tissue sealing and cutting procedure. At least some of the devices 1a-1n/2a-2m may be employed to identify pathology, such as the effects of diseases, using the cloud-based computing to examine data including images of samples of body tissue for diagnostic purposes. This may include localization and margin confirmation of tissue and phenotypes. At least some of the devices 1a-1n/2a-2m may be employed to identify anatomical structures of the body using a variety of sensors integrated with imaging devices and techniques such as overlaying images captured by multiple imaging devices. The data gathered by the devices 1a-1n/2a-2m, including image data, may be transferred to the cloud computing system 20064 or the local computer system 20063 or both for data processing and manipulation including image processing and manipulation. The data may be analyzed to improve surgical procedure outcomes by determining if further treatment, such as the application of endoscopic intervention, emerging technologies, a targeted radiation, targeted intervention, and precise robotics to tissue-specific sites and conditions, may be pursued. Such data analysis may further employ outcome analytics processing and using standardized approaches may provide beneficial feedback to either confirm surgical treatments and the behavior of the surgeon or suggest modifications to surgical treatments and the behavior of the surgeon.

Applying cloud computer data processing techniques on the measurement data collected by the sensing systems 20069, the surgical data network can provide improved surgical outcomes, improved recovery outcomes, reduced costs, and improved patient satisfaction. At least some of the sensing systems 20069 may be employed to assess physiological conditions of a surgeon operating on a patient or a patient being prepared for a surgical procedure or a patient recovering after a surgical procedure. The cloud-based computing system 20064 may be used to monitor biomarkers associated with a surgeon or a patient in real-time and to generate surgical plans based at least on measurement data gathered prior to a surgical procedure, provide control signals to the surgical instruments during a surgical procedure, notify a patient of a complication during post-surgical period.

The operating theater devices 1a-1n may be connected to the modular communication hub 20065 over a wired channel or a wireless channel depending on the configuration of the devices 1a-1n to a network hub 20061. The network hub 20061 may be implemented, in one aspect, as a local network broadcast device that works on the physical layer of the Open System Interconnection (OSI) model. The network hub may provide connectivity to the devices 1a-1n located in the same operating theater network. The network hub 20061 may collect data in the form of packets and sends them to the router in half duplex mode. The network hub 20061 may not store any media access control/Internet Protocol (MAC/IP) to transfer the device data. Only one of the devices 1a-1n can send data at a time through the network hub 20061. The network hub 20061 may not have routing tables or intelligence regarding where to send information and broadcasts all network data across each connection and to a remote server 20067 of the cloud computing system 20064. The network hub 20061 can detect basic network errors such as collisions but having all information broadcast to multiple ports can be a security risk and cause bottlenecks.

The operating theater devices 2a-2m may be connected to a network switch 20062 over a wired channel or a wireless channel. The network switch 20062 works in the data link layer of the OSI model. The network switch 20062 may be a multicast device for connecting the devices 2a-2m located in the same operating theater to the network. The network switch 20062 may send data in the form of frames to the network router 20066 and may work in full duplex mode. Multiple devices 2a-2m can send data at the same time through the network switch 20062. The network switch 20062 stores and uses MAC addresses of the devices 2a-2m to transfer data.

The network hub 20061 and/or the network switch 20062 may be coupled to the network router 20066 for connection to the cloud computing system 20064. The network router 20066 works in the network layer of the OSI model. The network router 20066 creates a route for transmitting data packets received from the network hub 20061 and/or network switch 20062 to cloud-based computer resources for further processing and manipulation of the data collected by any one of or all the devices 1a-1n/2a-2m and wearable sensing system 20011. The network router 20066 may be employed to connect two or more different networks located in different locations, such as, for example, different operating theaters of the same healthcare facility or different networks located in different operating theaters of different healthcare facilities. The network router 20066 may send data in the form of packets to the cloud computing system 20064 and works in full duplex mode. Multiple devices can send data at the same time. The network router 20066 may use IP addresses to transfer data.

In an example, the network hub 20061 may be implemented as a USB hub, which allows multiple USB devices to be connected to a host computer. The USB hub may expand a single USB port into several tiers so that there are more ports available to connect devices to the host system computer. The network hub 20061 may include wired or wireless capabilities to receive information over a wired channel or a wireless channel. In one aspect, a wireless USB short-range, high-bandwidth wireless radio communication protocol may be employed for communication between the devices 1a-1n and devices 2a-2m located in the operating theater.

In examples, the operating theater devices 1a-1n/2a-2m and/or the sensing systems 20069 may communicate to the modular communication hub 20065 via Bluetooth wireless technology standard for exchanging data over short distances (using short-wavelength UHF radio waves in the ISM band from 2.4 to 2.485 GHz) from fixed and mobile devices and building personal area networks (PANs). The operating theater devices 1a-1n/2a-2m and/or the sensing systems 20069 may communicate to the modular communication hub 20065 via a number of wireless or wired communication standards or protocols, including but not limited to Bluetooth, Low-Energy Bluetooth, near-field communication (NFC), Wi-Fi (IEEE 802.11 family), WiMAX (IEEE 802.16 family), IEEE 802.20, new radio (NR), long-term evolution (LTE), and Ev-DO, HSPA+, HSDPA+, HSUPA+, EDGE, GSM, GPRS, CDMA, IDMA, DECT, and Ethernet derivatives thereof, as well as any other wireless and wired protocols that are designated as 3G, 4G, 5G, and beyond. The computing module may include a plurality of communication modules. For instance, a first communication module may be dedicated to shorter-range wireless communications such as Wi-Fi and Bluetooth Low-Energy Bluetooth, Bluetooth Smart, and a second communication module may be dedicated to longer-range wireless communications such as GPS, EDGE, GPRS, CDMA, WiMAX, LTE, Ev-DO, HSPA+, HSDPA+, HSUPA+, EDGE, GSM, GPRS, CDMA, TDMA, and others.

The modular communication hub 20065 may serve as a central connection for one or more of the operating theater devices 1a-1n/2a-2m and/or the sensing systems 20069 and may handle a data type known as frames. Frames may carry the data generated by the devices 1a-1n/2a-2m and/or the sensing systems 20069. When a frame is received by the modular communication hub 20065, it may be amplified and/or sent to the network router 20066, which may transfer the data to the cloud computing system 20064 or the local computer system 20063 by using a number of wireless or wired communication standards or protocols, as described herein.

The modular communication hub 20065 can be used as a standalone device or be connected to compatible network hubs 20061 and network switches 20062 to form a larger network. The modular communication hub 20065 can be generally easy to install, configure, and maintain, making it a good option for networking the operating theater devices 1a-1n/2a-2m.

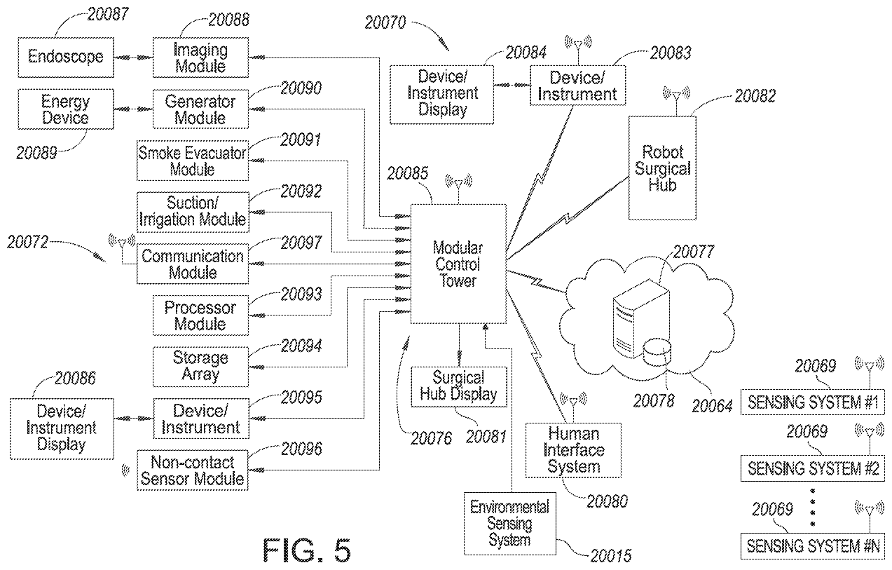

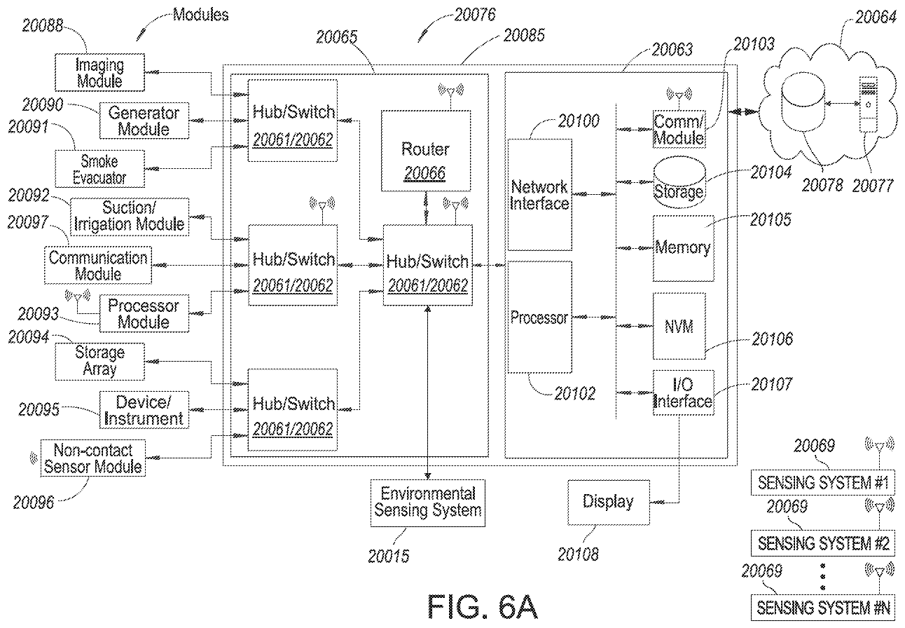

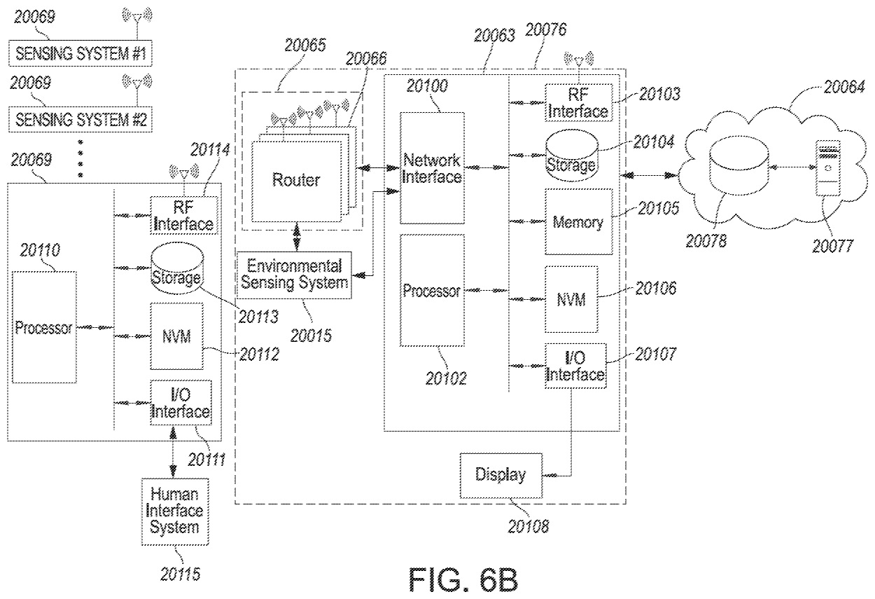

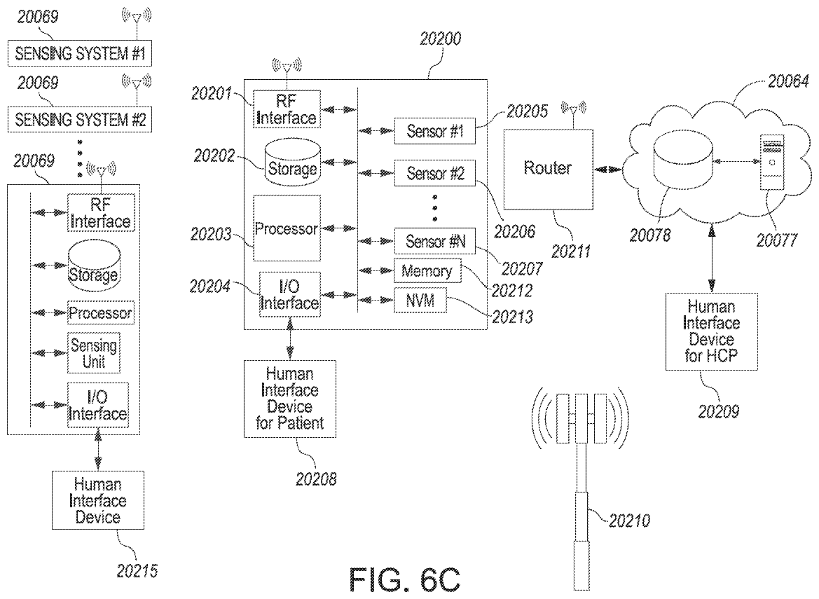

FIG. 5 illustrates a computer-implemented interactive surgical system 20070 that may be a part of the surgeon monitoring system 20002. The computer-implemented interactive surgical system 20070 is similar in many respects to the surgeon sensing system 20002. For example, the computer-implemented interactive surgical system 20070 may include one or more surgical sub-systems 20072, which are similar in many respects to the surgeon monitoring systems 20002. Each sub-surgical system 20072 includes at least one surgical hub 20076 in communication with a cloud computing system 20064 that may include a remote server 20077 and a remote storage 20078. In one aspect, the computer-implemented interactive surgical system 20070 may include a modular control tower 20085 connected to multiple operating theater devices such as sensing systems (e.g., surgeon sensing systems 20002 and/or patient sensing system 20003), intelligent surgical instruments, robots, and other computerized devices located in the operating theater. As shown in FIG. 6A, the modular control tower 20085 may include a modular communication hub 20065 coupled to a local computing system 20063.

As illustrated in the example of FIG. 5, the modular control tower 20085 may be coupled to an imaging module 20088 that may be coupled to an endoscope 20087, a generator module 20090 that may be coupled to an energy device 20089, a smoke evacuator module 20091, a suction/irrigation module 20092, a communication module 20097, a processor module 20093, a storage array 20094, a smart device/instrument 20095 optionally coupled to a display 20086 and 20084 respectively, and a non-contact sensor module 20096. The modular control tower 20085 may also be in communication with one or more sensing systems 20069 and an environmental sensing system 20015. The sensing systems 20069 may be connected to the modular control tower 20085 either directly via a router or via the communication module 20097. The operating theater devices may be coupled to cloud computing resources and data storage via the modular control tower 20085. A robot surgical hub 20082 also may be connected to the modular control tower 20085 and to the cloud computing resources. The devices/instruments 20095 or 20084, human interface system 20080, among others, may be coupled to the modular control tower 20085 via wired or wireless communication standards or protocols, as described herein. The human interface system 20080 may include a display sub-system and a notification sub-system. The modular control tower 20085 may be coupled to a hub display 20081 (e.g., monitor, screen) to display and overlay images received from the imaging module 20088, device/instrument display 20086, and/or other human interface systems 20080. The hub display 20081 also may display data received from devices connected to the modular control tower 20085 in conjunction with images and overlaid images.

FIG. 6A illustrates a surgical hub 20076 comprising a plurality of modules coupled to the modular control tower 20085. As shown in FIG. 6A, the surgical hub 20076 may be connected to a generator module 20090, the smoke evacuator module 20091, suction/irrigation module 20092, and the communication module 20097. The modular control tower 20085 may comprise a modular communication hub 20065, e.g., a network connectivity device, and a computer system 20063 to provide local wireless connectivity with the sensing systems, local processing, complication monitoring, visualization, and imaging, for example. As shown in FIG. 6A, the modular communication hub 20065 may be connected in a configuration (e.g., a tiered configuration) to expand a number of modules (e.g., devices) and a number of sensing systems 20069 that may be connected to the modular communication hub 20065 and transfer data associated with the modules and/or measurement data associated with the sensing systems 20069 to the computer system 20063, cloud computing resources, or both. As shown in FIG. 6A, each of the network hubs/switches 20061/20062 in the modular communication hub 20065 may include three downstream ports and one upstream port. The upstream network hub/switch may be connected to a processor 20102 to provide a communication connection to the cloud computing resources and a local display 20108. At least one of the network/hub switches 20061/20062 in the modular communication hub 20065 may have at least one wireless interface to provided communication connection between the sensing systems 20069 and/or the devices 20095 and the cloud computing system 20064. Communication to the cloud computing system 20064 may be made either through a wired or a wireless communication channel.

The surgical hub 20076 may employ a non-contact sensor module 20096 to measure the dimensions of the operating theater and generate a map of the surgical theater using either ultrasonic or laser-type non-contact measurement devices. An ultrasound-based non-contact sensor module may scan the operating theater by transmitting a burst of ultrasound and receiving the echo when it bounces off the perimeter walls of an operating theater as described under the heading “Surgical Hub Spatial Awareness Within an Operating Room” in U.S. Provisional Patent Application Ser. No. 62/611,341, titled INTERACTIVE SURGICAL PLATFORM, filed Dec. 28, 2017, which is herein incorporated by reference in its entirety, in which the sensor module is configured to determine the size of the operating theater and to adjust Bluetooth-pairing distance limits. A laser-based non-contact sensor module may scan the operating theater by transmitting laser light pulses, receiving laser light pulses that bounce off the perimeter walls of the operating theater, and comparing the phase of the transmitted pulse to the received pulse to determine the size of the operating theater and to adjust Bluetooth pairing distance limits, for example.

The computer system 20063 may comprise a processor 20102 and a network interface 20100. The processor 20102 may be coupled to a communication module 20103, storage 20104, memory 20105, non-volatile memory 20106, and input/output (I/O) interface 20107 via a system bus. The system bus can be any of several types of bus structure(s) including the memory bus or memory controller, a peripheral bus or external bus, and or a local bus using any variety of available bus architectures including, but not limited to, 9-bit bus, Industrial Standard Architecture (ISA), Micro-Charmel Architecture (MSA), Extended ISA (EISA), Intelligent Drive Electronics (IDE), VESA Local Bus (VLB), Peripheral Component Interconnect (PCI), USB, Advanced Graphics Port (AGP), Personal Computer Memory Card International Association bus (PCMCIA), Small Computer Systems Interface (SCSI), or any other proprietary bus.

The processor 20102 may be any single-core or multicore processor such as those known under the trade name ARM Cortex by Texas Instruments. In one aspect, the processor may be an LM4F230H5QR ARM Cortex-M4F Processor Core, available from Texas Instruments, for example, comprising an on-chip memory of 256 KB single-cycle flash memory, or other non-volatile memory, up to 40 MHz, a prefetch buffer to improve performance above 40 MHz, a 32 KB single-cycle serial random access memory (SRAM), an internal read-only memory (ROM) loaded with StellarisWare® software, a 2 KB electrically erasable programmable read-only memory (EEPROM), and/or one or more pulse width modulation (PWM) modules, one or more quadrature encoder inputs (QEI) analogs, one or more 12-bit analog-to-digital converters (ADCs) with 12 analog input channels, details of which are available for the product datasheet.

In an example, the processor 20102 may comprise a safety controller comprising two controller-based families such as TMS570 and RM4x, known under the trade name Hercules ARM Cortex R4, also by Texas Instruments. The safety controller may be configured specifically for IEC 61508 and ISO 26262 safety critical applications, among others, to provide advanced integrated safety features while delivering scalable performance, connectivity, and memory options.

The system memory may include volatile memory and non-volatile memory. The basic input/output system (BIOS), containing the basic routines to transfer information between elements within the computer system, such as during start-up, is stored in non-volatile memory. For example, the non-volatile memory can include ROM, programmable ROM (PROM), electrically programmable ROM (EPROM), EEPROM, or flash memory. Volatile memory includes random-access memory (RAM), which acts as external cache memory. Moreover, RAM is available in many forms such as SRAM, dynamic RAM (DRAM), synchronous DRAM (SDRAM), double data rate SDRAM (DDR SDRAM), enhanced SDRAM (ESDRAM), Synchlink DRAM (SLDRAM), and direct Rambus RAM (DRRAM).

The computer system 20063 also may include removable/non-removable, volatile/non-volatile computer storage media, such as for example disk storage. The disk storage can include, but is not limited to, devices like a magnetic disk drive, floppy disk drive, tape drive, Jaz drive, Zip drive, LS-60 drive, flash memory card, or memory stick. In addition, the disk storage can include storage media separately or in combination with other storage media including, but not limited to, an optical disc drive such as a compact disc ROM device (CD-ROM), compact disc recordable drive (CD-R Drive), compact disc rewritable drive (CD-RW Drive), or a digital versatile disc ROM drive (DVD-ROM). To facilitate the connection of the disk storage devices to the system bus, a removable or non-removable interface may be employed.

It is to be appreciated that the computer system 20063 may include software that acts as an intermediary between users and the basic computer resources described in a suitable operating environment. Such software may include an operating system. The operating system, which can be stored on the disk storage, may act to control and allocate resources of the computer system. System applications may take advantage of the management of resources by the operating system through program modules and program data stored either in the system memory or on the disk storage. It is to be appreciated that various components described herein can be implemented with various operating systems or combinations of operating systems.

A user may enter commands or information into the computer system 20063 through input device(s) coupled to the I/O interface 20107. The input devices may include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processor 20102 through the system bus via interface port(s). The interface port(s) include, for example, a serial port, a parallel port, a game port, and a USB. The output device(s) use some of the same types of ports as input device(s). Thus, for example, a USB port may be used to provide input to the computer system 20063 and to output information from the computer system 20063 to an output device. An output adapter may be provided to illustrate that there can be some output devices like monitors, displays, speakers, and printers, among other output devices that may require special adapters. The output adapters may include, by way of illustration and not limitation, video and sound cards that provide a means of connection between the output device and the system bus. It should be noted that other devices and/or systems of devices, such as remote computer(s), may provide both input and output capabilities.

The computer system 20063 can operate in a networked environment using logical connections to one or more remote computers, such as cloud computer(s), or local computers. The remote cloud computer(s) can be a personal computer, server, router, network PC, workstation, microprocessor-based appliance, peer device, or other common network node, and the like, and typically includes many or all of the elements described relative to the computer system. For purposes of brevity, only a memory storage device is illustrated with the remote computer(s). The remote computer(s) may be logically connected to the computer system through a network interface and then physically connected via a communication connection. The network interface may encompass communication networks such as local area networks (LANs) and wide area networks (WANs). LAN technologies may include Fiber Distributed Data Interface (FDDI), Copper Distributed Data Interface (CDDI), Ethernet/IEEE 802.3, Token Ring/IEEE 802.5, and the like. WAN technologies may include, but are not limited to, point-to-point links, circuit-switching networks like Integrated Services Digital Networks (ISDN) and variations thereon, packet-switching networks, and Digital Subscriber Lines (DSL).

In various examples, the computer system 20063 of FIG. 4, FIG. 6A and FIG. 6B, the imaging module 20088 and/or human interface system 20080, and/or the processor module 20093 of FIG. 5 and FIG. 6A may comprise an image processor, image-processing engine, media processor, or any specialized digital signal processor (DSP) used for the processing of digital images. The image processor may employ parallel computing with single instruction, multiple data (SIMD) or multiple instruction, multiple data (MIMD) technologies to increase speed and efficiency. The digital image-processing engine can perform a range of tasks. The image processor may be a system on a chip with multicore processor architecture.

The communication connection(s) may refer to the hardware/software employed to connect the network interface to the bus. While the communication connection is shown for illustrative clarity inside the computer system 20063, it can also be external to the computer system 20063. The hardware/software necessary for connection to the network interface may include, for illustrative purposes only, internal and external technologies such as modems, including regular telephone-grade modems, cable modems, optical fiber modems, and DSL modems, ISDN adapters, and Ethernet cards. In some examples, the network interface may also be provided using an RF interface.

FIG. 6B illustrates an example of a wearable monitoring system, e.g., a controlled patient monitoring system. A controlled patient monitoring system may be the sensing system used to monitor a set of patient biomarkers when the patient is at a healthcare facility. The controlled patient monitoring system may be deployed for pre-surgical patient monitoring when a patient is being prepared for a surgical procedure, in-surgical monitoring when a patient is being operated on, or in post-surgical monitoring, for example, when a patient is recovering, etc. As illustrated in FIG. 6B, a controlled patient monitoring system may include a surgical hub system 20076, which may include one or more routers 20066 of the modular communication hub 20065 and a computer system 20063. The routers 20065 may include wireless routers, wired switches, wired routers, wired or wireless networking hubs, etc. In an example, the routers 20065 may be part of the infrastructure. The computing system 20063 may provide local processing for monitoring various biomarkers associated with a patient or a surgeon, and a notification mechanism to indicate to the patient and/or a healthcare provided (HCP) that a milestone (e.g., a recovery milestone) is met or a complication is detected. The computing system 20063 of the surgical hub system 20076 may also be used to generate a severity level associated with the notification, for example, a notification that a complication has been detected.

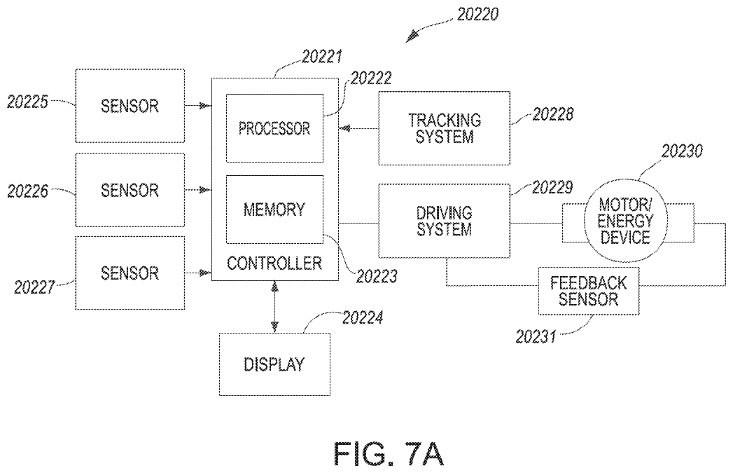

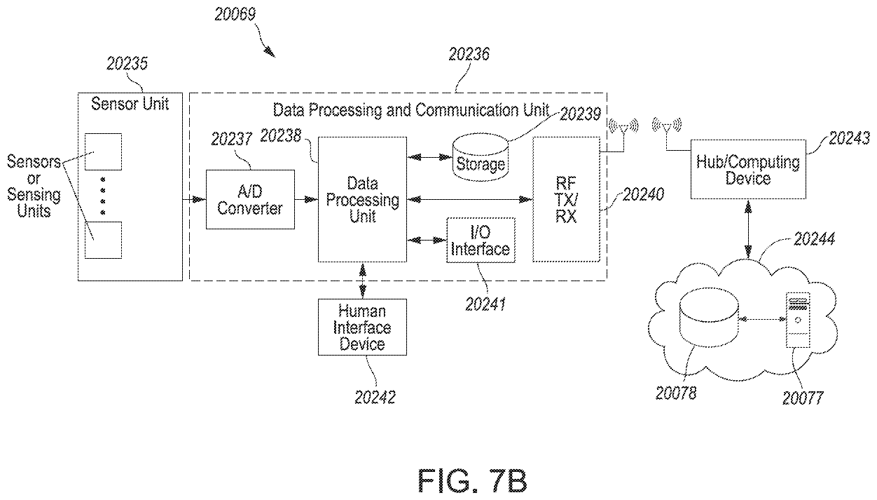

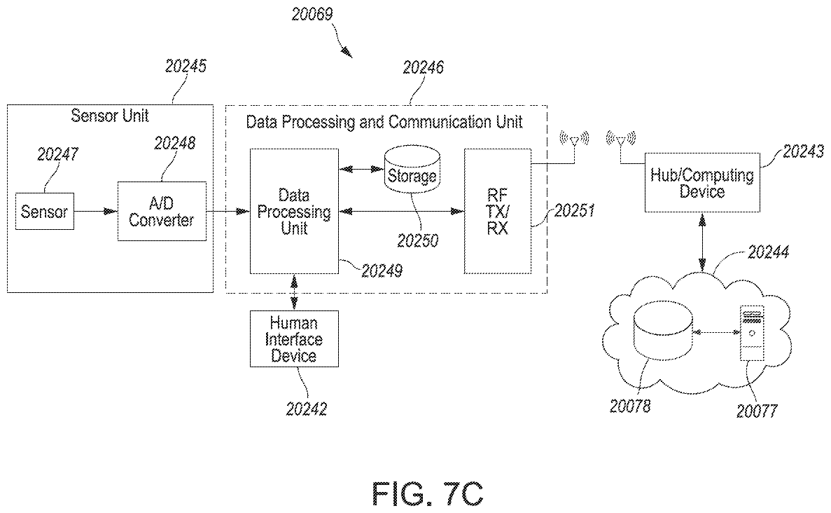

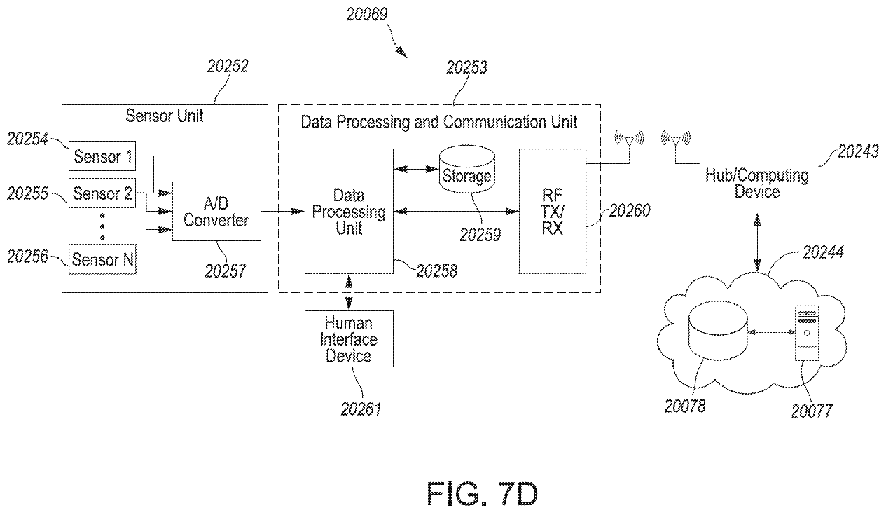

The computing system 20063 of FIG. 4, FIG. 6B, the computing device 20200 of FIG. 6C, the hub/computing device 20243 of FIG. 7B, FIG. 7C, or FIG. 7D may be a surgical computing system or a hub device, a laptop, a tablet, a smart phone, etc.

As shown in FIG. 6B, a set of sensing systems 20069 and/or an environmental sensing system 20015 (as described in FIG. 2A) may be connected to the surgical hub system 20076 via the routers 20065. The routers 20065 may also provide a direct communication connection between the sensing systems 20069 and the cloud computing system 20064, for example, without involving the local computer system 20063 of the surgical hub system 20076. Communication from the surgical hub system 20076 to the cloud 20064 may be made either through a wired or a wireless communication channel.

As shown in FIG. 6B, the computer system 20063 may include a processor 20102 and a network interface 20100. The processor 20102 may be coupled to a radio frequency (RF) interface or a communication module 20103, storage 20104, memory 20105, non-volatile memory 20106, and input/output interface 20107 via a system bus, as described in FIG. 6A. The computer system 20063 may be connected with a local display unit 20108. In some examples, the display unit 20108 may be replaced by a HID. Details about the hardware and software components of the computer system are provided in FIG. 6A.

As shown in FIG. 6B, a sensing system 20069 may include a processor 20110. The processor 20110 may be coupled to a radio frequency (RF) interface 20114, storage 20113, memory (e.g., a non-volatile memory) 20112, and I/O interface 20111 via a system bus. The system bus can be any of several types of bus structure(s) including the memory bus or memory controller, a peripheral bus or external bus, and/or a local bus, as described herein. The processor 20110 may be any single-core or multicore processor as described herein.

It is to be appreciated that the sensing system 20069 may include software that acts as an intermediary between sensing system users and the computer resources described in a suitable operating environment. Such software may include an operating system. The operating system, which can be stored on the disk storage, may act to control and allocate resources of the computer system. System applications may take advantage of the management of resources by the operating system through program modules and program data stored either in the system memory or on the disk storage. It is to be appreciated that various components described herein can be implemented with various operating systems or combinations of operating systems.

The sensing system 20069 may be connected to a human interface system 20115. The human interface system 20115 may be a touch screen display. The human interface system 20115 may include a human interface display for displaying information associated with a surgeon biomarker and/or a patient biomarker, display a prompt for a user action by a patient or a surgeon, or display a notification to a patient or a surgeon indicating information about a recovery millstone or a complication. The human interface system 20115 may be used to receive input from a patient or a surgeon. Other human interface systems may be connected to the sensing system 20069 via the I/O interface 20111. For example, the human interface device 20115 may include devices for providing a haptic feedback as a mechanism for prompting a user to pay attention to a notification that may be displayed on a display unit.

The sensing system 20069 may operate in a networked environment using logical connections to one or more remote computers, such as cloud computer(s), or local computers. The remote cloud computer(s) can be a personal computer, server, router, network PC, workstation, microprocessor-based appliance, peer device, or other common network node, and the like, and typically includes many or all of the elements described relative to the computer system. The remote computer(s) may be logically connected to the computer system through a network interface. The network interface may encompass communication networks such as local area networks (LANs), wide area networks (WANs), and/or mobile networks. LAN technologies may include Fiber Distributed Data Interface (FDDI), Copper Distributed Data Interface (CDDI), Ethernet/IEEE 802.3, Token Ring/IEEE 802.5, Wi-Fi/IEEE 802.11, and the like. WAN technologies may include, but are not limited to, point-to-point links, circuit-switching networks like Integrated Services Digital Networks (ISDN) and variations thereon, packet-switching networks, and Digital Subscriber Lines (DSL). The mobile networks may include communication links based on one or more of the following mobile communication protocols: GSM/GPRS/EDGE (2G), UMTS/HSPA (3G), long term evolution (LTE) or 4G, LTE-Advanced (LTE-A), new radio (NR) or 5G, etc.

FIG. 6C illustrates an exemplary uncontrolled patient monitoring system, for example, when the patient is away from a healthcare facility. The uncontrolled patient monitoring system may be used for pre-surgical patient monitoring when a patient is being prepared for a surgical procedure but is away from a healthcare facility, or in post-surgical monitoring, for example, when a patient is recovering away from a healthcare facility.

As illustrated in FIG. 6C, one or more sensing systems 20069 are in communication with a computing device 20200, for example, a personal computer, a laptop, a tablet, or a smart phone. The computing system 20200 may provide processing for monitoring of various biomarkers associated with a patient, a notification mechanism to indicate that a milestone (e.g., a recovery milestone) is met or a complication is detected. The computing system 20200 may also provide instructions for the user of the sensing system to follow. The communication between the sensing systems 20069 and the computing device 20200 may be established directly using a wireless protocol as described herein or via the wireless router/hub 20211.

As shown in FIG. 6C, the sensing systems 20069 may be connected to the computing device 20200 via router 20211. The router 20211 may include wireless routers, wired switches, wired routers, wired or wireless networking hubs, etc. The router 20211 may provide a direct communication connection between the sensing systems 20069 and the cloud servers 20064, for example, without involving the local computing device 20200. The computing device 20200 may be in communication with the cloud server 20064. For example, the computing device 20200 may be in communication with the cloud 20064 through a wired or a wireless communication channel. In an example, a sensing system 20069 may be in communication with the cloud directly over a cellular network, for example, via a cellular base station 20210.

As shown in FIG. 6C, the computing device 20200 may include a processor 20203 and a network or an RF interface 20201. The processor 20203 may be coupled to a storage 20202, memory 20212, non-volatile memory 20213, and input/output interface 20204 via a system bus, as described in FIG. 6A and FIG. 6B. Details about the hardware and software components of the computer system are provided in FIG. 6A. The computing device 20200 may include a set of sensors, for example, sensor #1 20205, sensor #2 20206 up to sensor #n 20207. These sensors may be a part of the computing device 20200 and may be used to measure one or more attributes associated with the patient. The attributes may provide a context about a biomarker measurement performed by one of the sensing systems 20069. For example, sensor #1 may be an accelerometer that may be used to measure acceleration forces in order to sense movement or vibrations associated with the patient. In an example, the sensors 20205 to 20207 may include one or more of: a pressure sensor, an altimeter, a thermometer, a lidar, or the like.

As shown in FIG. 6B, a sensing system 20069 may include a processor, a radio frequency interface, a storage, a memory or non-volatile memory, and input/output interface via a system bus, as described in FIG. 6A. The sensing system may include a sensor unit and a processing and communication unit, as described in FIG. 7B through 7D. The system bus can be any of several types of bus structure(s) including the memory bus or memory controller, a peripheral bus or external bus, and/or a local bus, as described herein. The processor may be any single-core or multicore processor, as described herein.

The sensing system 20069 may be in communication with a human interface system 20215.

The human interface system 20215 may be a touch screen display. The human interface system 20215 may be used to display information associated with a patient biomarker, display a prompt for a user action by a patient, or display a notification to a patient indicating information about a recovery millstone or a complication. The human interface system 20215 may be used to receive input from a patient. Other human interface systems may be connected to the sensing system 20069 via the I/O interface. For example, the human interface system may include devices for providing a haptic feedback as a mechanism for prompting a user to pay attention to a notification that may be displayed on a display unit. The sensing system 20069 may operate in a networked environment using logical connections to one or more remote computers, such as cloud computer(s), or local computers, as described in FIG. 6B.

FIG. 7A illustrates a logical diagram of a control system 20220 of a surgical instrument or a surgical tool in accordance with one or more aspects of the present disclosure. The surgical instrument or the surgical tool may be configurable. The surgical instrument may include surgical fixtures specific to the procedure at-hand, such as imaging devices, surgical staplers, energy devices, endocutter devices, or the like. For example, the surgical instrument may include any of a powered stapler, a powered stapler generator, an energy device, an advanced energy device, an advanced energy jaw device, an endocutter clamp, an energy device generator, an in-operating-room imaging system, a smoke evacuator, a suction-irrigation device, an insufflation system, or the like. The system 20220 may comprise a control circuit. The control circuit may include a microcontroller 20221 comprising a processor 20222 and a memory 20223. One or more of sensors 20225, 20226, 20227, for example, provide real-time feedback to the processor 20222. A motor 20230, driven by a motor driver 20229, operably couples a longitudinally movable displacement member to drive the I-beam knife element. A tracking system 20228 may be configured to determine the position of the longitudinally movable displacement member. The position information may be provided to the processor 20222, which can be programmed or configured to determine the position of the longitudinally movable drive member as well as the position of a firing member, firing bar, and I-beam knife element. Additional motors may be provided at the tool driver interface to control I-beam firing, closure tube travel, shaft rotation, and articulation. A display 20224 may display a variety of operating conditions of the instruments and may include touch screen functionality for data input. Information displayed on the display 20224 may be overlaid with images acquired via endoscopic imaging modules.

In one aspect, the microcontroller 20221 may be any single-core or multicore processor such as those known under the trade name ARM Cortex by Texas Instruments. In one aspect, the main microcontroller 20221 may be an LM4F230H5QR ARM Cortex-M4F Processor Core, available from Texas Instruments, for example, comprising an on-chip memory of 256 KB single-cycle flash memory, or other non-volatile memory, up to 40 MHz, a prefetch buffer to improve performance above 40 MHz, a 32 KB single-cycle SRAM, and internal ROM loaded with StellarisWare® software, a 2 KB EEPROM, one or more PWM modules, one or more QEI analogs, and/or one or more 12-bit ADCs with 12 analog input channels, details of which are available for the product datasheet.

In one aspect, the microcontroller 20221 may comprise a safety controller comprising two controller-based families such as TMS570 and RM4x, known under the trade name Hercules ARM Cortex R4, also by Texas Instruments. The safety controller may be configured specifically for IEC 61508 and ISO 26262 safety critical applications, among others, to provide advanced integrated safety features while delivering scalable performance, connectivity, and memory options.

The microcontroller 20221 may be programmed to perform various functions such as precise control over the speed and position of the knife and articulation systems. In one aspect, the microcontroller 20221 may include a processor 20222 and a memory 20223. The electric motor 20230 may be a brushed direct current (DC) motor with a gearbox and mechanical links to an articulation or knife system. In one aspect, a motor driver 20229 may be an A3941 available from Allegro Microsystems, Inc. Other motor drivers may be readily substituted for use in the tracking system 20228 comprising an absolute positioning system. A detailed description of an absolute positioning system is described in U.S. Patent Application Publication No. 2017/0296213, titled SYSTEMS AND METHODS FOR CONTROLLING A SURGICAL STAPLING AND CUTTING INSTRUMENT, which published on Oct. 19, 2017, which is herein incorporated by reference in its entirety.

The microcontroller 20221 may be programmed to provide precise control over the speed and position of displacement members and articulation systems. The microcontroller 20221 may be configured to compute a response in the software of the microcontroller 20221. The computed response may be compared to a measured response of the actual system to obtain an “observed” response, which is used for actual feedback decisions. The observed response may be a favorable, tuned value that balances the smooth, continuous nature of the simulated response with the measured response, which can detect outside influences on the system.

In some examples, the motor 20230 may be controlled by the motor driver 20229 and can be employed by the firing system of the surgical instrument or tool. In various forms, the motor 20230 may be a brushed DC driving motor having a maximum rotational speed of approximately 25,000 RPM. In some examples, the motor 20230 may include a brushless motor, a cordless motor, a synchronous motor, a stepper motor, or any other suitable electric motor. The motor driver 20229 may comprise an H-bridge driver comprising field-effect transistors (FETs), for example. The motor 20230 can be powered by a power assembly releasably mounted to the handle assembly or tool housing for supplying control power to the surgical instrument or tool. The power assembly may comprise a battery which may include a number of battery cells connected in series that can be used as the power source to power the surgical instrument or tool. In certain circumstances, the battery cells of the power assembly may be replaceable and/or rechargeable. In at least one example, the battery cells can be lithium-ion batteries which can be couplable to and separable from the power assembly.

The motor driver 20229 may be an A3941 available from Allegro Microsystems, Inc. A3941 may be a frill-bridge controller for use with external N-channel power metal-oxide semiconductor field-effect transistors (MOSFETs) specifically designed for inductive loads, such as brush DC motors. The driver 20229 may comprise a unique charge pump regulator that can provide full (>10 V) gate drive for battery voltages down to 7 V and can allow the A3941 to operate with a reduced gate drive, down to 5.5 V. A bootstrap capacitor may be employed to provide the above battery supply voltage required for N-channel MOSFETs. An internal charge pump for the high-side drive may allow DC (100% duty cycle) operation. The full bridge can be driven in fast or slow decay modes using diode or synchronous rectification. In the slow decay mode, current recirculation can be through the high-side or the low-side FETs. The power FETs may be protected from shoot-through by resistor-adjustable dead time. Integrated diagnostics provide indications of undervoltage, overtemperature, and power bridge faults and can be configured to protect the power MOSFETs under most short circuit conditions. Other motor drivers may be readily substituted for use in the tracking system 20228 comprising an absolute positioning system.

The tracking system 20228 may comprise a controlled motor drive circuit arrangement comprising a position sensor 20225 according to one aspect of this disclosure. The position sensor 20225 for an absolute positioning system may provide a unique position signal corresponding to the location of a displacement member. In some examples, the displacement member may represent a longitudinally movable drive member comprising a rack of drive teeth for meshing engagement with a corresponding drive gear of a gear reducer assembly. In some examples, the displacement member may represent the firing member, which could be adapted and configured to include a rack of drive teeth. In some examples, the displacement member may represent a firing bar or the I-beam, each of which can be adapted and configured to include a rack of drive teeth. Accordingly, as used herein, the term displacement member can be used generically to refer to any movable member of the surgical instrument or tool such as the drive member, the firing member, the firing bar, the I-beam, or any element that can be displaced. In one aspect, the longitudinally movable drive member can be coupled to the firing member, the firing bar, and the I-beam. Accordingly, the absolute positioning system can, in effect, track the linear displacement of the I-beam by tracking the linear displacement of the longitudinally movable drive member. In various aspects, the displacement member may be coupled to any position sensor 20225 suitable for measuring linear displacement. Thus, the longitudinally movable drive member, the firing member, the firing bar, or the I-beam, or combinations thereof, may be coupled to any suitable linear displacement sensor. Linear displacement sensors may include contact or non-contact displacement sensors. Linear displacement sensors may comprise linear variable differential transformers (LVDT), differential variable reluctance transducers (DVRT), a slide potentiometer, a magnetic sensing system comprising a movable magnet and a series of linearly arranged Hall effect sensors, a magnetic sensing system comprising a fixed magnet and a series of movable, linearly arranged Hall effect sensors, an optical sensing system comprising a movable light source and a series of linearly arranged photo diodes or photo detectors, an optical sensing system comprising a fixed light source and a series of movable linearly, arranged photodiodes or photodetectors, or any combination thereof.

The electric motor 20230 can include a rotatable shaft that operably interfaces with a gear assembly that is mounted in meshing engagement with a set, or rack, of drive teeth on the displacement member. A sensor element may be operably coupled to a gear assembly such that a single revolution of the position sensor 20225 element corresponds to some linear longitudinal translation of the displacement member. An arrangement of gearing and sensors can be connected to the linear actuator, via a rack and pinion arrangement, or a rotary actuator, via a spur gear or other connection. A power source may supply power to the absolute positioning system and an output indicator may display the output of the absolute positioning system. The displacement member may represent the longitudinally movable drive member comprising a rack of drive teeth formed thereon for meshing engagement with a corresponding drive gear of the gear reducer assembly. The displacement member may represent the longitudinally movable firing member, firing bar, I-beam, or combinations thereof.

A single revolution of the sensor element associated with the position sensor 20225 may be equivalent to a longitudinal linear displacement d1 of the of the displacement member, where d1 is the longitudinal linear distance that the displacement member moves from point “a” to point “b” after a single revolution of the sensor element coupled to the displacement member. The sensor arrangement may be connected via a gear reduction that results in the position sensor 20225 completing one or more revolutions for the full stroke of the displacement member. The position sensor 20225 may complete multiple revolutions for the full stroke of the displacement member.

A series of switches, where n is an integer greater than one, may be employed alone or in combination with a gear reduction to provide a unique position signal for more than one revolution of the position sensor 20225. The state of the switches may be fed back to the microcontroller 20221 that applies logic to determine a unique position signal corresponding to the longitudinal linear displacement d1+d2+ . . . dn of the displacement member. The output of the position sensor 20225 is provided to the microcontroller 20221. The position sensor 20225 of the sensor arrangement may comprise a magnetic sensor, an analog rotary sensor like a potentiometer, or an array of analog Hall-effect elements, which output a unique combination of position signals or values.

The position sensor 20225 may comprise any number of magnetic sensing elements, such as, for example, magnetic sensors classified according to whether they measure the total magnetic field or the vector components of the magnetic field. The techniques used to produce both types of magnetic sensors may encompass many aspects of physics and electronics. The technologies used for magnetic field sensing may include search coil, fluxgate, optically pumped, nuclear precession, SQUID, Hall-effect, anisotropic magnetoresistance, giant magnetoresistance, magnetic tunnel junctions, giant magnetoimpedance, magnetostrictive/piezoelectric composites, magnetodiode, magnetotransistor, fiber-optic, magneto-optic, and microelectromechanical systems-based magnetic sensors, among others.

In one aspect, the position sensor 20225 for the tracking system 20228 comprising an absolute positioning system may comprise a magnetic rotary absolute positioning system. The position sensor 20225 may be implemented as an AS5055EQFT single-chip magnetic rotary position sensor available from Austria Microsystems, AG. The position sensor 20225 is interfaced with the microcontroller 20221 to provide an absolute positioning system. The position sensor 20225 may be a low-voltage and low-power component and may include four Hall-effect elements in an area of the position sensor 20225 that may be located above a magnet. A high-resolution ADC and a smart power management controller may also be provided on the chip. A coordinate rotation digital computer (CORDIC) processor, also known as the digit-by-digit method and Volder's algorithm, may be provided to implement a simple and efficient algorithm to calculate hyperbolic and trigonometric functions that require only addition, subtraction, bit-shift, and table lookup operations. The angle position, alarm bits, and magnetic field information may be transmitted over a standard serial communication interface, such as a serial peripheral interface (SPI) interface, to the microcontroller 20221. The position sensor 20225 may provide 12 or 14 bits of resolution. The position sensor 20225 may be an AS5055 chip provided in a small QFN 16-pin 4×4×0.85 mm package.

The tracking system 20228 comprising an absolute positioning system may comprise and/or be programmed to implement a feedback controller, such as a PID, state feedback, and adaptive controller. A power source converts the signal from the feedback controller into a physical input to the system: in this case the voltage. Other examples include a PWM of the voltage, current, and force. Other sensor(s) may be provided to measure physical parameters of the physical system in addition to the position measured by the position sensor 20225. In some aspects, the other sensor(s) can include sensor arrangements such as those described in U.S. Pat. No. 9,345,481, titled STAPLE CARTRIDGE TISSUE THICKNESS SENSOR SYSTEM, which issued on May 24, 2016, which is herein incorporated by reference in its entirety; U.S. Patent Application Publication No. 2014/0263552, titled STAPLE CARTRIDGE TISSUE THICKNESS SENSOR SYSTEM, which published on Sep. 18, 2014, which is herein incorporated by reference in its entirety; and U.S. patent application Ser. No. 15/628,175, titled TECHNIQUES FOR ADAPTIVE CONTROL OF MOTOR VELOCITY OF A SURGICAL STAPLING AND CUTTING INSTRUMENT, filed Jun. 20, 2017, which is herein incorporated by reference in its entirety. In a digital signal processing system, an absolute positioning system is coupled to a digital data acquisition system where the output of the absolute positioning system will have a finite resolution and sampling frequency. The absolute positioning system may comprise a compare-and-combine circuit to combine a computed response with a measured response using algorithms, such as a weighted average and a theoretical control loop, that drive the computed response towards the measured response. The computed response of the physical system may take into account properties like mass, inertia, viscous friction, inductance resistance, etc., to predict what the states and outputs of the physical system will be by knowing the input.

The absolute positioning system may provide an absolute position of the displacement member upon power-up of the instrument, without retracting or advancing the displacement member to a reset (zero or home) position as may be required with conventional rotary encoders that merely count the number of steps forwards or backwards that the motor 20230 has taken to infer the position of a device actuator, drive bar, knife, or the like.

A sensor 20226, such as, for example, a strain gauge or a micro-strain gauge, may be configured to measure one or more parameters of the end effector, such as, for example, the amplitude of the strain exerted on the anvil during a clamping operation, which can be indicative of the closure forces applied to the anvil. The measured strain may be converted to a digital signal and provided to the processor 20222. Alternatively, or in addition to the sensor 20226, a sensor 20227, such as, for example, a load sensor, can measure the closure force applied by the closure drive system to the anvil. The sensor 20227, such as, for example, a load sensor, can measure the firing force applied to an I-beam in a firing stroke of the surgical instrument or tool. The I-beam is configured to engage a wedge sled, which is configured to upwardly cam staple drivers to force out staples into deforming contact with an anvil. The I-beam also may include a sharpened cutting edge that can be used to sever tissue as the I-beam is advanced distally by the firing bar. Alternatively, a current sensor 20231 can be employed to measure the current drawn by the motor 20230. The force required to advance the firing member can correspond to the current drawn by the motor 20230, for example. The measured force may be converted to a digital signal and provided to the processor 20222.

In one form, the strain gauge sensor 20226 can be used to measure the force applied to the tissue by the end effector. A strain gauge can be coupled to the end effector to measure the force on the tissue being treated by the end effector. A system for measuring forces applied to the tissue grasped by the end effector may comprise a strain gauge sensor 20226, such as, for example, a micro-strain gauge, that can be configured to measure one or more parameters of the end effector, for example. In one aspect, the strain gauge sensor 20226 can measure the amplitude or magnitude of the strain exerted on a jaw member of an end effector during a clamping operation, which can be indicative of the tissue compression. The measured strain can be converted to a digital signal and provided to a processor 20222 of the microcontroller 20221. A load sensor 20227 can measure the force used to operate the knife element, for example, to cut the tissue captured between the anvil and the staple cartridge. A magnetic field sensor can be employed to measure the thickness of the captured tissue. The measurement of the magnetic field sensor also may be converted to a digital signal and provided to the processor 20222.

The measurements of the tissue compression, the tissue thickness, and/or the force required to close the end effector on the tissue, as respectively measured by the sensors 20226, 20227, can be used by the microcontroller 20221 to characterize the selected position of the firing member and/or the corresponding value of the speed of the firing member. In one instance, a memory 20223 may store a technique, an equation, and/or a lookup table which can be employed by the microcontroller 20221 in the assessment.

The control system 20220 of the surgical instrument or tool also may comprise wired or wireless communication circuits to communicate with the modular communication hub 20065 as shown in FIG. 5 and FIG. 6A.

FIG. 7B shows an example sensing system 20069. The sensing system may be a surgeon sensing system or a patient sensing system. The sensing system 20069 may include a sensor unit 20235 and a human interface system 20242 that are in communication with a data processing and communication unit 20236. The data processing and communication unit 20236 may include an analog-to-digital converted 20237, a data processing unit 20238, a storage unit 20239, and an input/output interface 20241, a transceiver 20240. The sensing system 20069 may be in communication with a surgical hub or a computing device 20243, which in turn is in communication with a cloud computing system 20244. The cloud computing system 20244 may include a cloud storage system 20078 and one or more cloud servers 20077.

The sensor unit 20235 may include one or more ex vivo or in vivo sensors for measuring one or more biomarkers. The biomarkers may include, for example, Blood pH, hydration state, oxygen saturation, core body temperature, heart rate, Heart rate variability, Sweat rate, Skin conductance, Blood pressure, Light exposure, Environmental temperature, Respiratory rate, Coughing and sneezing, Gastrointestinal motility, Gastrointestinal tract imaging, Tissue perfusion pressure, Bacteria in respiratory tract, Alcohol consumption, Lactate (sweat), Peripheral temperature, Positivity and optimism, Adrenaline (sweat), Cortisol (sweat), Edema, Mycotoxins, VO2 max, Pre-operative pain, chemicals in the air, Circulating tumor cells, Stress and anxiety, Confusion and delirium, Physical activity, Autonomic tone, Circadian rhythm, Menstrual cycle, Sleep, etc. These biomarkers may be measured using one or more sensors, for example, photosensors (e.g., photodiodes, photoresistors), mechanical sensors (e.g., motion sensors), acoustic sensors, electrical sensors, electrochemical sensors, thermoelectric sensors, infrared sensors, etc. The sensors may measure the biomarkers as described herein using one of more of the following sensing technologies: photoplethysmography, electrocardiography, electroencephalography, colorimetry, impedimentary, potentiometry, amperometry, etc.

As illustrated in FIG. 7B, a sensor in the sensor unit 20235 may measure a physiological signal (e.g., a voltage, a current, a PPG signal, etc.) associated with a biomarker to be measured. The physiological signal to be measured may depend on the sensing technology used, as described herein. The sensor unit 20235 of the sensing system 20069 may be in communication with the data processing and communication unit 20236. In an example, the sensor unit 20235 may communicate with the data processing and communication unit 20236 using a wireless interface. The data processing and communication unit 20236 may include an analog-to-digital converter (ADC) 20237, a data processing unit 20238, a storage 20239, an I/O interface 20241, and an RF transceiver 20240. The data processing unit 20238 may include a processor and a memory unit.

The sensor unit 20235 may transmit the measured physiological signal to the ADC 20237 of the data processing and communication unit 20236. In an example, the measured physiological signal may be passed through one or more filters (e.g., an RC low-pass filter) before being sent to the ADC. The ADC may convert the measured physiological signal into measurement data associated with the biomarker. The ADC may pass measurement data to the data processing unit 20238 for processing. In an example, the data processing unit 20238 may send the measurement data associated with the biomarker to a surgical hub or a computing device 20243, which in turn may send the measurement data to a cloud computing system 20244 for further processing. The data processing unit may send the measurement data to the surgical hub or the computing device 20243 using one of the wireless protocols, as described herein. In an example, the data processing unit 20238 may first process the raw measurement data received from the sensor unit and send the processed measurement data to the surgical hub or a computing device 20243.

In an example, the data processing and communication unit 20236 of the sensing system 20069 may receive a threshold value associated with a biomarker for monitoring from a surgical hub, a computing device 20243, or directly from a cloud server 20077 of the cloud computing system 20244. The data processing unit 20236 may compare the measurement data associated with the biomarker to be monitored with the corresponding threshold value received from the surgical hub, the computing device 20243, or the cloud server 20077. The data processing and communication unit 20236 may send a notification message to the HID 20242 indicating that a measurement data value has crossed the threshold value. The notification message may include the measurement data associated with the monitored biomarker. The data processing and computing unit 20236 may send a notification via a transmission to a surgical hub or a computing device 20243 using one of the following RF protocols: Bluetooth, Bluetooth Low-Energy (BLE), Bluetooth Smart, Zigbee, Z-wave, IPv6 Low-power wireless Personal Area Network (6LoWPAN), Wi-Fi. The data processing unit 20238 may send a notification (e.g., a notification for an HCP) directly to a cloud server via a transmission to a cellular transmission/reception point (TRP) or a base station using one or more of the following cellular protocols: GSM/GPRS/EDGE (2G), UMTS/HSPA (3G), long term evolution (LTE) or 4G, LTE-Advanced (LTE-A), new radio (NR) or 5G. In an example, the sensing unit may be in communication with the hub/computing device via a router, as described in FIG. 6A through FIG. 6C.

FIG. 7C shows an example sensing system 20069 (e.g., a surgeon sensing system or a patient sensing system). The sensing system 20069 may include a sensor unit 20245, a data processing and communication unit 20246, and a human interface device 20242. The sensor unit 20245 may include a sensor 20247 and an analog-to-digital converted (ADC) 20248. The ADC 20248 in the sensor unit 20245 may convert a physiological signal measured by the sensor 20247 into measurement data associated with a biomarker. The sensor unit 20245 may send the measurement data to the data processing and communication unit 20246 for further processing. In an example, the sensor unit 20245 may send the measurement data to the data processing and communication unit 20246 using an inter-integrated circuit (I2C) interface.

The data processing and communication unit 20246 includes a data processing unit 20249, a storage unit 20250, and an RF transceiver 20251. The sensing system may be in communication with a surgical hub or a computing device 20243, which in turn may be in communication with a cloud computing system 20244. The cloud computing system 20244 may include a remote server 20077 and an associated remote storage 20078. The sensor unit 20245 may include one or more ex vivo or in vivo sensors for measuring one or more biomarkers, as described herein.

The data processing and communication unit 20246 after processing the measurement data received from the sensor unit 20245 may further process the measurement data and/or send the measurement data to the smart hub or the computing device 20243, as described in FIG. 7B. In an example, the data processing and communication unit 20246 may send the measurement data received from the sensor unit 20245 to the remote server 20077 of the cloud computing system 20244 for further processing and/or monitoring.

FIG. 7D shows an example sensing system 20069 (e.g., a surgeon sensing system or a patient sensing system). The sensing system 20069 may include a sensor unit 20252, a data processing and communication unit 20253, and a human interface system 20261. The sensor unit 20252 may include a plurality of sensors 20254, 20255 up to 20256 to measure one or more physiological signals associated with a patient or surgeon's biomarkers and/or one or more physical state signals associated with physical state of a patient or a surgeon. The sensor unit 20252 may also include one or more analog-to-digital converter(s) (ADCs) 20257. A list of biomarkers may include biomarkers such as those biomarkers disclosed herein. The ADC(s) 20257 in the sensor unit 20252 may convert each of the physiological signals and/or physical state signals measured by the sensors 20254-20256 into respective measurement data. The sensor unit 20252 may send the measurement data associated with one or more biomarkers as well as with the physical state of a patient or a surgeon to the data processing and communication unit 20253 for further processing. The sensor unit 20252 may send the measurement data to the data processing and communication unit 20253 individually for each of the sensors Sensor 1 20254 to Sensor N 20256 or combined for all the sensors. In an example, the sensor unit 20252 may send the measurement data to the data processing and communication unit 20253 via an I2C interface.

The data processing and communication unit 20253 may include a data processing unit 20258, a storage unit 20259, and an RF transceiver 20260. The sensing system 20069 may be in communication with a surgical hub or a computing device 20243, which in turn is in communication with a cloud computing system 20244 comprising at least one remote server 20077 and at least one storage unit 20078. The sensor units 20252 may include one or more ex vivo or in vivo sensors for measuring one or more biomarkers, as described herein.

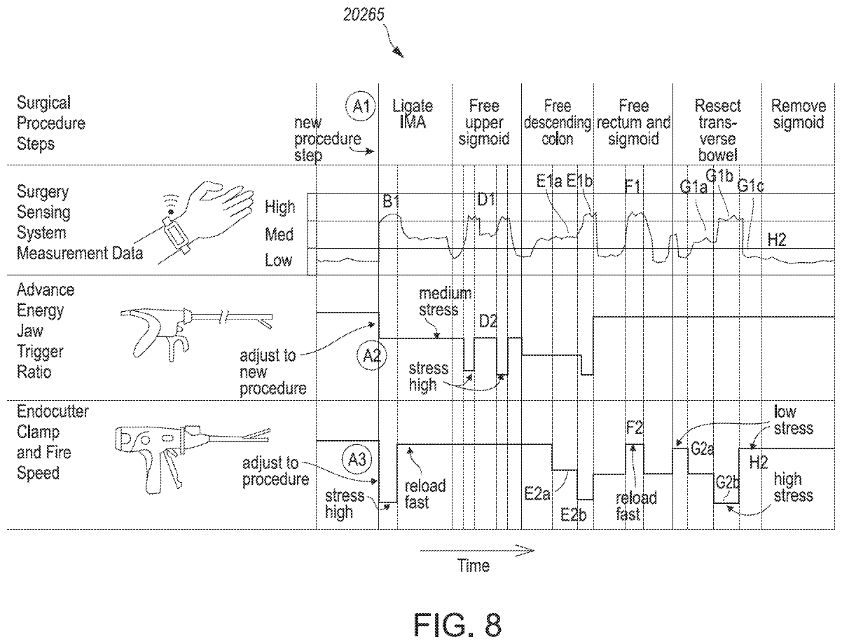

FIG. 8 is an example of using a surgical task situational awareness and measurement data from one or more surgeon sensing systems to adjust surgical instrument controls. FIG. 8 illustrates a timeline 20265 of an illustrative surgical procedure and the contextual information that a surgical hub can derive from data received from one or more surgical devices, one or more surgeon sensing systems, and/or one or more environmental sensing systems at each step in the surgical procedure. The devices that could be controlled by a surgical hub may include advanced energy devices, endocutter clamps, etc. The surgeon sensing systems may include sensing systems for measuring one or more biomarkers associated with the surgeon, for example, heart rate, sweat composition, respiratory rate, etc. The environmental sensing system may include systems for measuring one or more of the environmental attributes, for example, cameras for detecting a surgeon's position/movements/breathing pattern, spatial microphones, for example to measure ambient noise in the surgical theater and/or the tone of voice of a healthcare provider, temperature/humidity of the surroundings, etc.

In the following description of the timeline 20265 illustrated in FIG. 8, reference should also be made to FIG. 5. FIG. 5 provides various components used in a surgical procedure. The timeline 20265 depicts the steps that may be taken individually and/or collectively by the nurses, surgeons, and other medical personnel during the course of an exemplary colorectal surgical procedure. In a colorectal surgical procedure, a situationally aware surgical hub 20076 may receive data from various data sources throughout the course of the surgical procedure, including data generated each time a healthcare provider (HCP) utilizes a modular device/instrument 20095 that is paired with the surgical hub 20076. The surgical hub 20076 may receive this data from the paired modular devices 20095. The surgical hub may receive measurement data from sensing systems 20069. The surgical hub may use the data from the modular device/instruments 20095 and/or measurement data from the sensing systems 20069 to continually derive inferences (i.e., contextual information) about an HCP's stress level and the ongoing procedure as new data is received, such that the stress level of the surgeon relative to the step of the procedure that is being performed is obtained. The situational awareness system of the surgical hub 20076 may perform one or more of the following: record data pertaining to the procedure for generating reports, verify the steps being taken by the medical personnel, provide data or prompts (e.g., via a display screen) that may be pertinent for the particular procedural step, adjust modular devices based on the context (e.g., activate monitors, adjust the FOV of the medical imaging device, change the energy level of an ultrasonic surgical instrument or RF electrosurgical instrument), or take any other such action described herein. In an example, these steps may be performed by a remote server 20077 of a cloud system 20064 and communicated with the surgical hub 20076.

As a first step (not shown in FIG. 8 for brevity), the hospital staff members may retrieve the patient's EMR from the hospital's EMR database. Based on select patient data in the EMR, the surgical hub 20076 may determine that the procedure to be performed is a colorectal procedure. The staff members may scan the incoming medical supplies for the procedure. The surgical hub 20076 may cross-reference the scanned supplies with a list of supplies that can be utilized in various types of procedures and confirms that the mix of supplies corresponds to a colorectal procedure. The surgical hub 20076 may pair each of the sensing systems 20069 worn by different HCPs.

Once each of the devices is ready and pre-surgical preparation is complete, the surgical team may begin by making incisions and place trocars. The surgical team may perform access and prep by dissecting adhesions, if any, and identifying inferior mesenteric artery (IMA) branches. The surgical hub 20076 can infer that the surgeon is in the process of dissecting adhesions, at least based on the data it may receive from the RF or ultrasonic generator indicating that an energy instrument is being fired. The surgical hub 20076 may cross-reference the received data with the retrieved steps of the surgical procedure to determine that an energy instrument being fired at this point in the process (e.g., after the completion of the previously discussed steps of the procedure) corresponds to the dissection step.

After dissection, the HCP may proceed to the ligation step (e.g., indicated by A1) of the procedure. As illustrated in FIG. 8, the HCP may begin by ligating the IMA. The surgical hub 20076 may infer that the surgeon is ligating arteries and veins because it may receive data from the advanced energy jaw device and/or the endocutter indicating that the instrument is being fired. The surgical hub may also receive measurement data from one of the HCP's sensing systems indicating higher stress level of the HCP (e.g., indicated by B1 mark on the time axis). For example, higher stress level may be indicated by change in the HCP's heart rate from a base value. The surgical hub 20076, like the prior step, may derive this inference by cross-referencing the receipt of data from the surgical stapling and cutting instrument with the retrieved steps in the process (e.g., as indicated by A2 and A3). The surgical hub 20076 may monitor the advance energy jaw trigger ratio and/or the endocutter clamp and firing speed during the high stress time periods. In an example, the surgical hub 20076 may send an assistance control signal to the advanced energy jaw device and/or the endocutter device to control the device in operation. The surgical hub may send the assistance signal based on the stress level of the HCP that is operating the surgical device and/or situational awareness known to the surgical hub. For example, the surgical hub 20076 may send control assistance signals to an advanced energy device or an endocutter clamp, as indicated in FIG. 8 by A2 and A3.

The HCP may proceed to the next step of freeing the upper sigmoid followed by freeing descending colon, rectum, and sigmoid. The surgical hub 20076 may continue to monitor the high stress markers of the HCP (e.g., as indicated by D1, E1a, E1b, F1). The surgical hub 20076 may send assistance signals to the advanced energy jaw device and/or the endocutter device during the high stress time periods, as illustrated in FIG. 8.

After mobilizing the colon, the HCP may proceed with the segmentectomy portion of the procedure. For example, the surgical hub 20076 may infer that the HCP is transecting the bowel and sigmoid removal based on data from the surgical stapling and cutting instrument, including data from its cartridge. The cartridge data can correspond to the size or type of staple being fired by the instrument, for example. As different types of staples are utilized for different types of tissues, the cartridge data can thus indicate the type of tissue being stapled and/or transected. It should be noted that surgeons regularly switch back and forth between surgical stapling/cutting instruments and surgical energy (e.g., RF or ultrasonic) instruments depending upon the step in the procedure because different instruments are better adapted for particular tasks. Therefore, the sequence in which the stapling/cutting instruments and surgical energy instruments are used can indicate what step of the procedure the surgeon is performing.

The surgical hub may determine and send a control signal to surgical device based on the stress level of the HCP. For example, during time period G1b, a control signal G2b may be sent to an endocutter clamp. Upon removal of the sigmoid, the incisions are closed, and the post-operative portion of the procedure may begin. The patient's anesthesia can be reversed. The surgical hub 20076 may infer that the patient is emerging from the anesthesia based on one or more sensing systems attached to the patient.

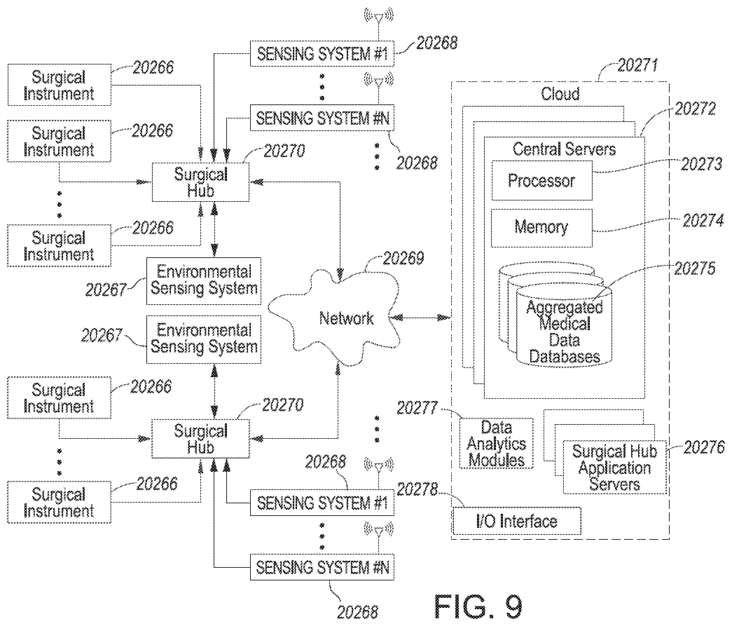

FIG. 9 is a block diagram of the computer-implemented interactive surgical system with surgeon/patient monitoring, in accordance with at least one aspect of the present disclosure. In one aspect, the computer-implemented interactive surgical system may be configured to monitor surgeon biomarkers and/or patient biomarkers using one or more sensing systems 20069. The surgeon biomarkers and/or the patient biomarkers may be measured before, after, and/or during a surgical procedure. In one aspect, the computer-implemented interactive surgical system may be configured to monitor and analyze data related to the operation of various surgical systems 20069 that include surgical hubs, surgical instruments, robotic devices and operating theaters or healthcare facilities. The computer-implemented interactive surgical system may include a cloud-based analytics system. The cloud-based analytics system may include one or more analytics servers.

As illustrated in FIG. 9, the cloud-based monitoring and analytics system may comprise a plurality of sensing systems 20268 (may be the same or similar to the sensing systems 20069), surgical instruments 20266 (may be the same or similar to instruments 20031), a plurality of surgical hubs 20270 (may be the same or similar to hubs 20006), and a surgical data network 20269 (may be the same or similar to the surgical data network described in FIG. 4) to couple the surgical hubs 20270 to the cloud 20271 (may be the same or similar to cloud computing system 20064). Each of the plurality of surgical hubs 20270 may be communicatively coupled to one or more surgical instruments 20266. Each of the plurality of surgical hubs 20270 may also be communicatively coupled to the one or more sensing systems 20268, and the cloud 20271 of the computer-implemented interactive surgical system via the network 20269. The surgical hubs 20270 and the sensing systems 20268 may be communicatively coupled using wireless protocols as described herein. The cloud system 20271 may be a remote centralized source of hardware and software for storing, processing, manipulating, and communicating measurement data from the sensing systems 20268 and data generated based on the operation of various surgical systems 20268.

As shown in FIG. 9, access to the cloud system 20271 may be achieved via the network 20269, which may be the Internet or some other suitable computer network. Surgical hubs 20270 that may be coupled to the cloud system 20271 can be considered the client side of the cloud computing system (e.g., cloud-based analytics system). Surgical instruments 20266 may be paired with the surgical hubs 20270 for control and implementation of various surgical procedures and/or operations, as described herein. Sensing systems 20268 may be paired with surgical hubs 20270 for in-surgical surgeon monitoring of surgeon related biomarkers, pre-surgical patient monitoring, in-surgical patient monitoring, or post-surgical monitoring of patient biomarkers to track and/or measure various milestones and/or detect various complications. Environmental sensing systems 20267 may be paired with surgical hubs 20270 measuring environmental attributes associated with a surgeon or a patient for surgeon monitoring, pre-surgical patient monitoring, in-surgical patient monitoring, or post-surgical monitoring of patient.

Surgical instruments 20266, environmental sensing systems 20267, and sensing systems 20268 may comprise wired or wireless transceivers for data transmission to and from their corresponding surgical hubs 20270 (which may also comprise transceivers). Combinations of one or more of surgical instruments 20266, sensing systems 20268, or surgical hubs 20270 may indicate particular locations, such as operating theaters, intensive care unit (ICU) rooms, or recovery rooms in healthcare facilities (e.g., hospitals), for providing medical operations, pre-surgical preparation, and/or post-surgical recovery. For example, the memory of a surgical hub 20270 may store location data.

As shown in FIG. 9, the cloud system 20271 may include one or more central servers 20272 (may be same or similar to remote server 20067), surgical hub application servers 20276, data analytics modules 20277, and an input/output (“I/O”) interface 20278. The central servers 20272 of the cloud system 20271 may collectively administer the cloud computing system, which includes monitoring requests by client surgical hubs 20270 and managing the processing capacity of the cloud system 20271 for executing the requests. Each of the central servers 20272 may comprise one or more processors 20273 coupled to suitable memory devices 20274 which can include volatile memory such as random-access memory (RAM) and non-volatile memory such as magnetic storage devices. The memory devices 20274 may comprise machine executable instructions that when executed cause the processors 20273 to execute the data analytics modules 20277 for the cloud-based data analysis, real-time monitoring of measurement data received from the sensing systems 20268, operations, recommendations, and other operations as described herein. The processors 20273 can execute the data analytics modules 20277 independently or in conjunction with hub applications independently executed by the hubs 20270. The central servers 20272 also may comprise aggregated medical data databases 20275, which can reside in the memory 20274.

Based on connections to various surgical hubs 20270 via the network 20269, the cloud 20271 can aggregate data from specific data generated by various surgical instruments 20266 and/or monitor real-time data from sensing systems 20268 and the surgical hubs 20270 associated with the surgical instruments 20266 and/or the sensing systems 20268. Such aggregated data from the surgical instruments 20266 and/or measurement data from the sensing systems 20268 may be stored within the aggregated medical databases 20275 of the cloud 20271. In particular, the cloud 20271 may advantageously track real-time measurement data from the sensing systems 20268 and/or perform data analysis and operations on the measurement data and/or the aggregated data to yield insights and/or perform functions that individual hubs 20270 could not achieve on their own. To this end, as shown in FIG. 9, the cloud 20271 and the surgical hubs 20270 are communicatively coupled to transmit and receive information. The I/O interface 20278 is connected to the plurality of surgical hubs 20270 via the network 20269. In this way, the I/O interface 20278 can be configured to transfer information between the surgical hubs 20270 and the aggregated medical data databases 20275. Accordingly, the I/O interface 20278 may facilitate read/write operations of the cloud-based analytics system. Such read/write operations may be executed in response to requests from hubs 20270. These requests could be transmitted to the surgical hubs 20270 through the hub applications. The I/O interface 20278 may include one or more high speed data ports, which may include universal serial bus (USB) ports, IEEE 1394 ports, as well as Wi-Fi and Bluetooth I/O interfaces for connecting the cloud 20271 to surgical hubs 20270. The hub application servers 20276 of the cloud 20271 may be configured to host and supply shared capabilities to software applications (e.g., hub applications) executed by surgical hubs 20270. For example, the hub application servers 20276 may manage requests made by the hub applications through the hubs 20270, control access to the aggregated medical data databases 20275, and perform load balancing.

The cloud computing system configuration described in the present disclosure may be designed to address various issues arising in the context of medical operations (e.g., pre-surgical monitoring, in-surgical monitoring, and post-surgical monitoring) and procedures performed using medical devices, such as the surgical instruments 20266, 20031. In particular, the surgical instruments 20266 may be digital surgical devices configured to interact with the cloud 20271 for implementing techniques to improve the performance of surgical operations. The sensing systems 20268 may be systems with one or more sensors that are configured to measure one or more biomarkers associated with a surgeon perfuming a medical operation and/or a patient on whom a medical operation is planned to be performed, is being performed or has been performed. Various surgical instruments 20266, sensing systems 20268, and/or surgical hubs 20270 may include human interface systems (e.g., having a touch-controlled user interfaces) such that clinicians and/or patients may control aspects of interaction between the surgical instruments 20266 or the sensing system 20268 and the cloud 20271. Other suitable user interfaces for control such as auditory controlled user interfaces may also be used.

The cloud computing system configuration described in the present disclosure may be designed to address various issues arising in the context of monitoring one or more biomarkers associated with a healthcare professional (HCP) or a patient in pre-surgical, in-surgical, and post-surgical procedures using sensing systems 20268. Sensing systems 20268 may be surgeon sensing systems or patient sensing systems configured to interact with the surgical hub 20270 and/or with the cloud system 20271 for implementing techniques to monitor surgeon biomarkers and/or patient biomarkers. Various sensing systems 20268 and/or surgical hubs 20270 may comprise touch-controlled human interface systems such that the HCPs or the patients may control aspects of interaction between the sensing systems 20268 and the surgical hub 20270 and/or the cloud systems 20271. Other suitable user interfaces for control such as auditory controlled user interfaces may also be used.

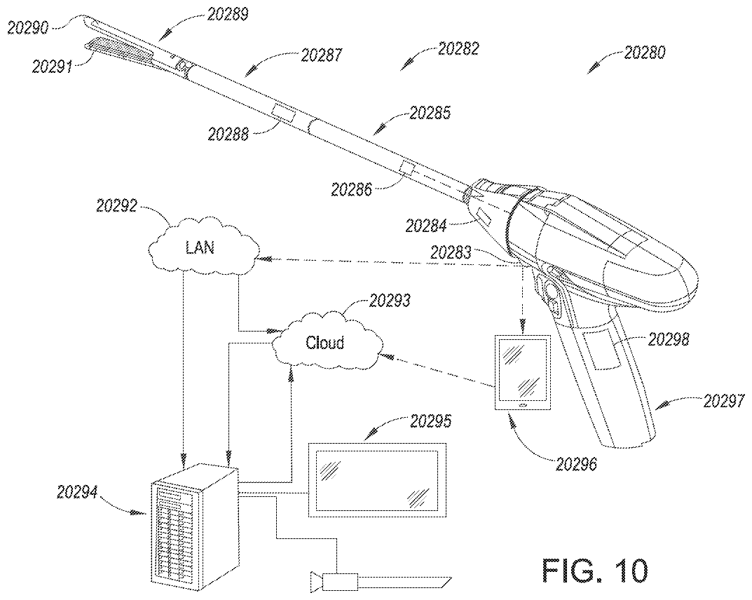

FIG. 10 illustrates an example surgical system 20280 in accordance with the present disclosure and may include a surgical instrument 20282 that can be in communication with a console 20294 or a portable device 20296 through a local area network 20292 or a cloud network 20293 via a wired or wireless connection. In various aspects, the console 20294 and the portable device 20296 may be any suitable computing device. The surgical instrument 20282 may include a handle 20297, an adapter 20285, and a loading unit 20287. The adapter 20285 releasably couples to the handle 20297 and the loading unit 20287 releasably couples to the adapter 20285 such that the adapter 20285 transmits a force from a drive shaft to the loading unit 20287. The adapter 20285 or the loading unit 20287 may include a force gauge (not explicitly shown) disposed therein to measure a force exerted on the loading unit 20287. The loading unit 20287 may include an end effector 20289 having a first jaw 20291 and a second jaw 20290. The loading unit 20287 may be an in-situ loaded or multi-firing loading unit (MFLU) that allows a clinician to fire a plurality of fasteners multiple times without requiring the loading unit 20287 to be removed from a surgical site to reload the loading unit 20287.

The first and second jaws 20291, 20290 may be configured to clamp tissue therebetween, fire fasteners through the clamped tissue, and sever the clamped tissue. The first jaw 20291 may be configured to fire at least one fastener a plurality of times or may be configured to include a replaceable multi-fire fastener cartridge including a plurality of fasteners (e.g., staples, clips, etc.) that may be fired more than one time prior to being replaced. The second jaw 20290 may include an anvil that deforms or otherwise secures the fasteners, as the fasteners are ejected from the multi-fire fastener cartridge.

The handle 20297 may include a motor that is coupled to the drive shaft to affect rotation of the drive shaft. The handle 20297 may include a control interface to selectively activate the motor. The control interface may include buttons, switches, levers, sliders, touchscreen, and any other suitable input mechanisms or user interfaces, which can be engaged by a clinician to activate the motor.

The control interface of the handle 20297 may be in communication with a controller 20298 of the handle 20297 to selectively activate the motor to affect rotation of the drive shafts. The controller 20298 may be disposed within the handle 20297 and may be configured to receive input from the control interface and adapter data from the adapter 20285 or loading unit data from the loading unit 20287. The controller 20298 may analyze the input from the control interface and the data received from the adapter 20285 and/or loading unit 20287 to selectively activate the motor. The handle 20297 may also include a display that is viewable by a clinician during use of the handle 20297. The display may be configured to display portions of the adapter or loading unit data before, during, or after firing of the instrument 20282.

The adapter 20285 may include an adapter identification device 20284 disposed therein and the loading unit 20287 may include a loading unit identification device 20288 disposed therein. The adapter identification device 20284 may be in communication with the controller 20298, and the loading unit identification device 20288 may be in communication with the controller 20298. It will be appreciated that the loading unit identification device 20288 may be in communication with the adapter identification device 20284, which relays or passes communication from the loading unit identification device 20288 to the controller 20298.

The adapter 20285 may also include a plurality of sensors 20286 (one shown) disposed thereabout to detect various conditions of the adapter 20285 or of the environment (e.g., if the adapter 20285 is connected to a loading unit, if the adapter 20285 is connected to a handle, if the drive shafts are rotating, the torque of the drive shafts, the strain of the drive shafts, the temperature within the adapter 20285, a number of firings of the adapter 20285, a peak force of the adapter 20285 during firing, a total amount of force applied to the adapter 20285, a peak retraction force of the adapter 20285, a number of pauses of the adapter 20285 during firing, etc.). The plurality of sensors 20286 may provide an input to the adapter identification device 20284 in the form of data signals. The data signals of the plurality of sensors 20286 may be stored within or be used to update the adapter data stored within the adapter identification device 20284. The data signals of the plurality of sensors 20286 may be analog or digital. The plurality of sensors 20286 may include a force gauge to measure a force exerted on the loading unit 20287 during firing.

The handle 20297 and the adapter 20285 can be configured to interconnect the adapter identification device 20284 and the loading unit identification device 20288 with the controller 20298 via an electrical interface. The electrical interface may be a direct electrical interface (i.e., include electrical contacts that engage one another to transmit energy and signals therebetween). Additionally, or alternatively, the electrical interface may be a non-contact electrical interface to wirelessly transmit energy and signals therebetween (e.g., inductively transfer). It is also contemplated that the adapter identification device 20284 and the controller 20298 may be in wireless communication with one another via a wireless connection separate from the electrical interface.

The handle 20297 may include a transceiver 20283 that is configured to transmit instrument data from the controller 20298 to other components of the system 20280 (e.g., the LAN 20292, the cloud 20293, the console 20294, or the portable device 20296). The controller 20298 may also transmit instrument data and/or measurement data associated with one or more sensors 20286 to a surgical hub 20270, as illustrated in FIG. 9. The transceiver 20283 may receive data (e.g., cartridge data, loading unit data, adapter data, or other notifications) from the surgical hub 20270. The transceiver 20283 may receive data (e.g., cartridge data, loading unit data, or adapter data) from the other components of the system 20280. For example, the controller 20298 may transmit instrument data including a serial number of an attached adapter (e.g., adapter 20285) attached to the handle 20297, a serial number of a loading unit (e.g., loading unit 20287) attached to the adapter 20285, and a serial number of a multi-fire fastener cartridge loaded into the loading unit to the console 20294. Thereafter, the console 20294 may transmit data (e.g., cartridge data, loading unit data, or adapter data) associated with the attached cartridge, loading unit, and adapter, respectively, back to the controller 20298. The controller 20298 can display messages on the local instrument display or transmit the message, via transceiver 20283, to the console 20294 or the portable device 20296 to display the message on the display 20295 or portable device screen, respectively.

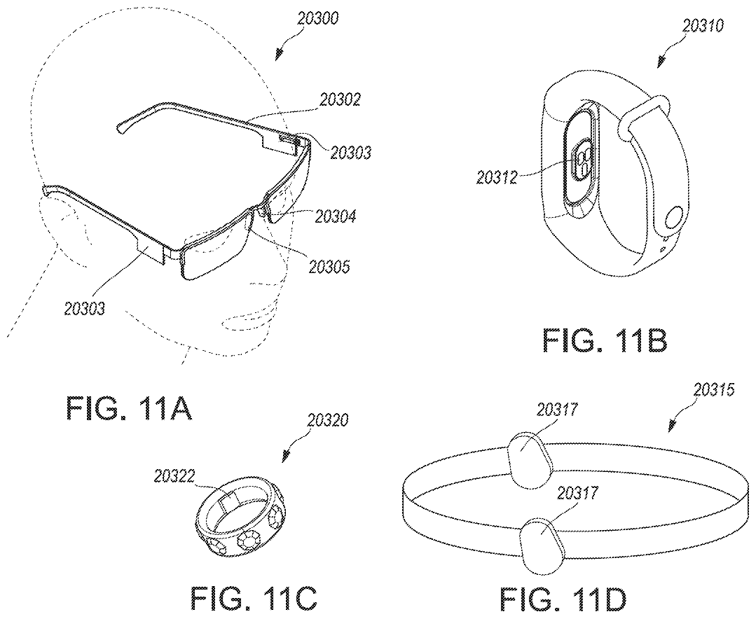

FIG. 11A to FIG. 11D illustrates examples of wearable sensing systems, e.g., surgeon sensing systems or patient sensing systems. FIG. 11A is an example of eyeglasses-based sensing system 20300 that may be based on an electrochemical sensing platform. The sensing system 20300 may be capable of monitoring (e.g., real-time monitoring) of sweat electrolytes and/or metabolites using multiple sensors 20304 and 20305 that are in contact with the surgeon's or patient's skin. For example, the sensing system 20300 may use an amperometry based biosensor 20304 and/or a potentiometry based biosensor 20305 integrated with the nose bridge pads of the eyeglasses 20302 to measure current and/or the voltage.

The amperometric biosensor 20304 may be used to measure sweat lactate levels (e.g., in mmol/L). Lactate that is a product of lactic acidosis that may occur due to decreased tissue oxygenation, which may be caused by sepsis or hemorrhage. A patient's lactate levels (e.g., >2 mmol/L) may be used to monitor the onset of sepsis, for example, during post-surgical monitoring. The potentiometric biosensor 20305 may be used to measure potassium levels in the patient's sweat. A voltage follower circuit with an operational amplifier may be used for measuring the potential signal between the reference and the working electrodes. The output of the voltage follower circuit may be filtered and converted into a digital value using an ADC.

The amperometric sensor 20304 and the potentiometric sensor 20305 may be connected to circuitries 20303 placed on each of the arms of the eyeglasses. The electrochemical sensors may be used for simultaneous real-time monitoring of sweat lactate and potassium levels. The electrochemical sensors may be screen printed on stickers and placed on each side of the glasses nose pads to monitor sweat metabolites and electrolytes. The electronic circuitries 20303 placed on the arms of the glasses frame may include a wireless data transceiver (e.g., a low energy Bluetooth transceiver) that may be used to transmit the lactate and/or potassium measurement data to a surgical hub or an intermediary device that may then forward the measurement data to the surgical hub. The eyeglasses-based sensing system 20300 may use signal conditioning unit to filter and amplify the electrical signal generated from the electrochemical sensors 20305 or 20304, a microcontroller to digitize the analog signal, and a wireless (e.g., a low energy Bluetooth) module to transfer the data to a surgical hub or a computing device, for example, as described in FIGS. 7B through 7D.

FIG. 11B is an example of a wristband-type sensing system 20310 comprising a sensor assembly 20312 (e.g., Photoplethysmography (PPG)-based sensor assembly or Electrocardiogram (ECG) based-sensor assembly). For example, in the sensing system 20310, the sensor assembly 20312 may collect and analyze arterial pulse in the wrist. The sensor assembly 20312 may be used to measure one or more biomarkers (e.g., heart rate, heart rate variability (HRV), etc.). In case of a sensing system with a PPG-based sensor assembly 20312, light (e.g., green light) may be passed through the skin. A percentage of the green light may be absorbed by the blood vessels and some of the green light may be reflected and detected by a photodetector. These differences or reflections are associated with the variations in the blood perfusion of the tissue and the variations may be used in detecting the heart-related information of the cardiovascular system (e.g., heart rate). For example, the amount of absorption may vary depending on the blood volume. The sensing system 20310 may determine the heart rate by measuring light reflectance as a function of time. HRV may be determined as the time period variation (e.g., standard deviation) between the steepest signal gradient prior to a peak, known as inter-beat intervals (IBIs).

In the case of a sensing system with an ECG-based sensor assembly 20312, a set of electrodes may be placed in contact with skin. The sensing system 20310 may measure voltages across the set of electrodes placed on the skin to determine heart rate. HRV in this case may be measured as the time period variation (e.g., standard deviation) between R peaks in the QRS complex, known as R-R intervals.

The sensing system 20310 may use a signal conditioning unit to filter and amplify the analog PPG signal, a microcontroller to digitize the analog PPG signal, and a wireless (e.g., a Bluetooth) module to transfer the data to a surgical hub or a computing device, for example, as described in FIGS. 7B through 7D.

FIG. 11C is an example ring sensing system 20320. The ring sensing system 20320 may include a sensor assembly (e.g., a heart rate sensor assembly) 20322. The sensor assembly 20322 may include a light source (e.g., red or green light emitting diodes (LEDs)), and photodiodes to detect reflected and/or absorbed light. The LEDs in the sensor assembly 20322 may shine light through a finger and the photodiode in the sensor assembly 20322 may measure heart rate and/or oxygen level in the blood by detecting blood volume change. The ring sensing system 20320 may include other sensor assemblies to measure other biomarkers, for example, a thermistor or an infrared thermometer to measure the surface body temperature. The ring sensing system 20320 may use a signal conditioning unit to filter and amplify the analog PPG signal, a microcontroller to digitize the analog PPG signal, and a wireless (e.g., a low energy Bluetooth) module to transfer the data to a surgical hub or a computing device, for example, as described in FIGS. 7B through 7D.

FIG. 11D is an example of an electroencephalogram (EEG) sensing system 20315. As illustrated in FIG. 11D, the sensing system 20315 may include one or more EEG sensor units 20317. The EEG sensor units 20317 may include a plurality of conductive electrodes placed in contact with the scalp. The conductive electrodes may be used to measure small electrical potentials that may arise outside of the head due to neuronal action within the brain. The EEG sensing system 20315 may measure a biomarker, for example, delirium by identifying certain brain patterns, for example, a slowing or dropout of the posterior dominant rhythm and loss of reactivity to eyes opening and closing. The ring sensing system 20315 may have a signal conditioning unit for filtering and amplifying the electrical potentials, a microcontroller to digitize the electrical signals, and a wireless (e.g., a low energy Bluetooth) module to transfer the data to a smart device, for example, as described in FIGS. 7B through 7D.

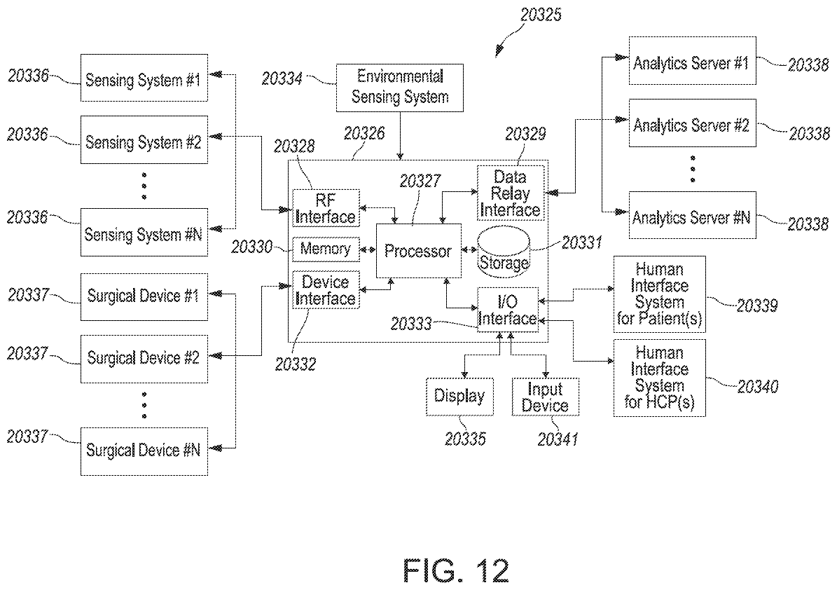

FIG. 12 illustrates a block diagram of a computer-implemented patient/surgeon monitoring system 20325 for monitoring one or more patient or surgeon biomarkers prior to, during, and/or after a surgical procedure. As illustrated in FIG. 12, one or more sensing systems 20336 may be used to measure and monitor the patient biomarkers, for example, to facilitate patient preparedness before a surgical procedure, and recovery after a surgical procedure. Sensing systems 20336 may be used to measure and monitor the surgeon biomarkers in real-time, for example, to assist surgical tasks by communicating relevant biomarkers (e.g., surgeon biomarkers) to a surgical hub 20326 and/or the surgical devices 20337 to adjust their function. The surgical device functions that may be adjusted may include power levels, advancement speeds, closure speed, loads, wait times, or other tissue dependent operational parameters. The sensing systems 20336 may also measure one or more physical attributes associated with a surgeon or a patient. The patient biomarkers and/or the physical attributes may be measured in real time.

The computer-implemented wearable patient/surgeon wearable sensing system 20325 may include a surgical hub 20326, one or more sensing systems 20336, and one or more surgical devices 20337. The sensing systems and the surgical devices may be communicably coupled to the surgical hub 20326. One or more analytics servers 20338, for example part of an analytics system, may also be communicably coupled to the surgical hub 20326. Although a single surgical hub 20326 is depicted, it should be noted that the wearable patient/surgeon wearable sensing system 20325 may include any number of surgical hubs 20326, which can be connected to form a network of surgical hubs 20326 that are communicably coupled to one or more analytics servers 20338, as described herein.

In an example, the surgical hub 20326 may be a computing device. The computing device may be a personal computer, a laptop, a tablet, a smart mobile device, etc. In an example, the computing device may be a client computing device of a cloud-based computing system. The client computing device may be a thin client.

In an example, the surgical hub 20326 may include a processor 20327 coupled to a memory 20330 for executing instructions stored thereon, a storage 20331 to store one or more databases such as an EMR database, and a data relay interface 20329 through which data is transmitted to the analytics servers 20338. In an example, the surgical hub 20326 further may include an I/O interface 20333 having an input device 20341 (e.g., a capacitive touchscreen or a keyboard) for receiving inputs from a user and an output device 20335 (e.g., a display screen) for providing outputs to a user. In an example, the input device and the output device may be a single device. Outputs may include data from a query input by the user, suggestions for products or a combination of products to use in a given procedure, and/or instructions for actions to be carried out before, during, and/or after a surgical procedure. The surgical hub 20326 may include a device interface 20332 for communicably coupling the surgical devices 20337 to the surgical hub 20326. In one aspect, the device interface 20332 may include a transceiver that may enable one or more surgical devices 20337 to connect with the surgical hub 20326 via a wired interface or a wireless interface using one of the wired or wireless communication protocols described herein. The surgical devices 20337 may include, for example, powered staplers, energy devices or their generators, imaging systems, or other linked systems, for example, smoke evacuators, suction-irrigation devices, insufflation systems, etc.

In an example, the surgical hub 20326 may be communicably coupled to one or more surgeon and/or patient sensing systems 20336. The sensing systems 20336 may be used to measure and/or monitor, in real-time, various biomarkers associated with a surgeon performing a surgical procedure or a patient on whom a surgical procedure is being performed. A list of the patient/surgeon biomarkers measured by the sensing systems 20336 is provided herein. In an example, the surgical hub 20326 may be communicably coupled to an environmental sensing system 20334. The environmental sensing systems 20334 may be used to measure and/or monitor, in real-time, environmental attributes, for example, temperature/humidity in the surgical theater, surgeon movements, ambient noise in the surgical theater caused by the surgeon's and/or the patient's breathing pattern, etc.

When sensing systems 20336 and the surgical devices 20337 are connected to the surgical hub 20326, the surgical hub 20326 may receive measurement data associated with one or more patient biomarkers, physical state associated with a patient, measurement data associated with surgeon biomarkers, and/or physical state associated with the surgeon from the sensing systems 20336, for example, as illustrated in FIG. 7B through 7D. The surgical hub 20326 may associate the measurement data, e.g., related to a surgeon, with other relevant pre-surgical data and/or data from situational awareness system to generate control signals for controlling the surgical devices 20337, for example, as illustrated in FIG. 8.

In an example, the surgical hub 20326 may compare the measurement data from the sensing systems 20336 with one or more thresholds defined based on baseline values, pre-surgical measurement data, and/or in surgical measurement data. The surgical hub 20326 may compare the measurement data from the sensing systems 20336 with one or more thresholds in real-time. The surgical hub 20326 may generate a notification for displaying. The surgical hub 20326 may send the notification for delivery to a human interface system for patient 20339 and/or the human interface system for a surgeon or an HCP 20340, for example, if the measurement data crosses (e.g., is greater than or lower than) the defined threshold value. The determination whether the notification would be sent to one or more of the to the human interface system for patient 20339 and/or the human interface system for an HCP 2340 may be based on a severity level associated with the notification. The surgical hub 20326 may also generate a severity level associated with the notification for displaying. The severity level generated may be displayed to the patient and/or the surgeon or the HCP. In an example, the patient biomarkers to be measured and/or monitored (e.g., measured and/or monitored in real-time) may be associated with a surgical procedural step. For example, the biomarkers to be measured and monitored for transection of veins and arteries step of a thoracic surgical procedure may include blood pressure, tissue perfusion pressure, edema, arterial stiffness, collagen content, thickness of connective tissue, etc., whereas the biomarkers to be measured and monitored for lymph node dissection step of the surgical procedure may include monitoring blood pressure of the patient. In an example, data regarding postoperative complications could be retrieved from an EMR database in the storage 20331 and data regarding staple or incision line leakages could be directly detected or inferred by a situational awareness system. The surgical procedural outcome data can be inferred by a situational awareness system from data received from a variety of data sources, including the surgical devices 20337, the sensing systems 20336, and the databases in the storage 20331 to which the surgical hub 20326 is connected.

The surgical hub 20326 may transmit the measurement data and physical state data it received from the sensing systems 20336 and/or data associated with the surgical devices 20337 to analytics servers 20338 for processing thereon. Each of the analytics servers 20338 may include a memory and a processor coupled to the memory that may execute instructions stored thereon to analyze the received data. The analytics servers 20338 may be connected in a distributed computing architecture and/or utilize a cloud computing architecture. Based on this paired data, the analytics system 20338 may determine optimal and/or preferred operating parameters for the various types of modular devices, generate adjustments to the control programs for the surgical devices 20337, and transmit (or “push”) the updates or control programs to the one or more surgical devices 20337. For example, an analytics system 20338 may correlate the perioperative data it received from the surgical hub 20236 with the measurement data associated with a physiological state of a surgeon or an HCP and/or a physiological state of the patient. The analytics system 20338 may determine when the surgical devices 20337 should be controlled and send an update to the surgical hub 20326. The surgical hub 20326 may then forward the control program to the relevant surgical device 20337.

Additional detail regarding the computer-implemented wearable patient/surgeon wearable sensing system 20325, including the surgical hub 30326, one or more sensing systems 20336 and various surgical devices 20337 connectable thereto, are described in connection with FIG. 5 through FIG. 7D.

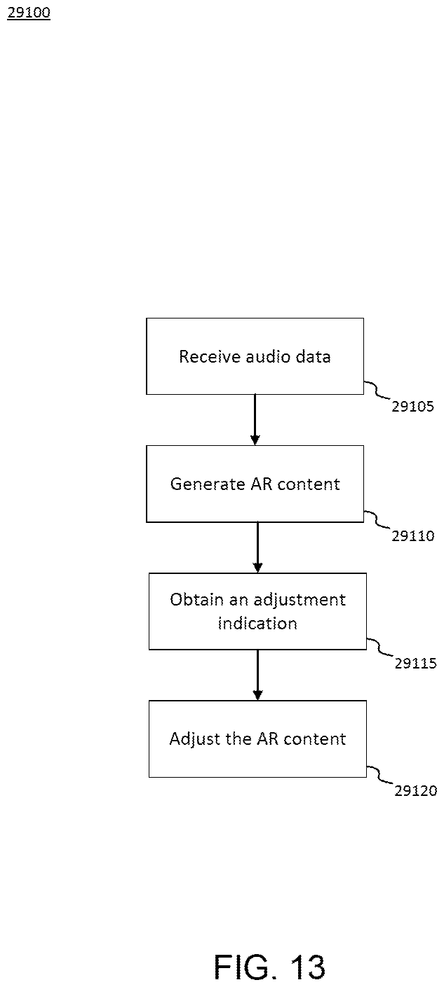

FIG. 13 illustrates an example flow of a computing system, such as an audio augmented reality (AR) computing system adjusting an AR content. In examples, an audio AR computing system may be or may include an earbud, a headset, a headphone, etc., or a computing system that controls the audio played via an earbud, a headset, a headphone, etc. The audio AR computing system may receive audio data. The audio data may be or may include one or more of audio data of measurement data associated with a user, ambient noise of the OR, audible feedback, audible information, and/or the like. The audio AR computing system may generate an AR content. For example, the audio AR computing system may generate the AR content based on the received audio data. The AR content may be or may include audible data, such as audible augmented feedback from one or more computing devices and/or other computing systems. The audio AR computing system may obtain an adjustment indication (e.g., from other computing system and/or a surgical computing system). The audio AR computing system may adjust the generated AR content based on the adjustment indication.

At 29105, an audio AR computing system may receive audio data from one or more sensing systems and/or computing system(s) in an operating room (OR). The audio data may be or may include audio data of measurement data associated with a user, ambient noise of the OR, audible feedback, audible information, and/or the like. The audio data may be adjusted, filtered and/or blocked. For example, the audio data may have filtered ambient noise of the OR.

At 29110, the audio AR computing system may generate AR content. For example, the audio AR computing system may generate the AR content based on the received audio data. The generated AR content may be or may include audible AR information. The audible AR may enhance what a user, such as a surgeon, hears. The audio AR computing system may allow a user to listen to the generated AR that may be or may include audible AR information associated with the received audio data.

Generating AR content is further described in U.S. patent application Ser. No. 17/062,509 (Atty Docket: END9287USNP16 titled INTERACTIVE INFORMATION OVERLAY ON MULTIPLE SURGICAL DISPLAYS, filed Oct. 2, 2020, which is incorporated by reference herein in its entirety.

At 29115, the audio AR computing system may obtain an adjustment indication. The adjustment indication may indicate adjustment information for the generated AR content. In examples, the adjustment indication may indicate to adjust the AR content by altering voice of the AR content and/or blocking noise (e.g., ambient noise) of the OR that may be included in the AR content. In examples, the adjustment indication may indicate to adjust an audio AR setting associated with an important surgical step and/or a critical surgical step. For example, the audio AR computing system may amplify and/or increase volume of the AR content (e.g., associated with the important surgical step and/or the critical surgical step). The audio AR computing system may reduce and/or decrease the volume of the other AR content (e.g., non-important surgical step and/or the non-critical surgical step). The audio AR computing system may increase or decrease frequency of transmitting the generated AR content to the user. In examples, if the audio AR computing system receives two or more audio data from other devices (e.g., other sensing systems and/or computing systems), the adjustment indication may indicate preferred audio data to be transmitted based on one or more of a preference of the user, priority information, and/or relevance to the current task and/or step of the operation. For example, the AR adjustment indication may be received from one or more computing systems (e.g., such as a surgical computing system and/or a surgical hub) in the OR.

At 29115, the audio AR computing system may adjust the generated AR content. In examples, based on the adjustment indication, the audio AR computing system may adjust the generated AR content. The audio AR computing system may alter the voice of the AR content. The audio AR computing system may block ambient noise of the OR. The audio AR computing system may amplify and/or increase volume of the AR content, reduce and/or reduce volume of the AR content, and/or increase or decrease frequency of transmitting the generated AR content to the user. The audio AR computing system may select audio data from two or more audio data from the sensing systems and/or the computing systems.

The audio AR computing system skip adjusting the generated AR content. For example, the audio AR computing system may allow the AR content (e.g., audible information) to pass through the AR content without filtering and/or adjusting. The audio AR computing system may determine (e.g., based on the adjustment indication) that a surgical operation is about to begin and/or a non-critical task and/or non-critical step of the surgical operation. The audio AR computing system may allow the AR content to pass through (e.g., skip adjusting) and allow the user, such as a surgeon, to listen to ambient noises of the OR.

In examples, the audio AR computing system may adjust the generated AR content by canceling and/or blocking ambient noises and/or other audible data, e.g., based on the adjustment indication. For example, the audio AR computing system may determine that an upcoming step (e.g., task) of a surgical operation is a critical step. The audio AR computing system may cancel and/or block the ambient noises of the OR and/or other audible data to provide a quiet environment for the user. The user of the audio AR computing system, such as a surgeon, may be focused on the critical step. The audio AR computing system may allow the user to experience a quiet environment, such as diminished interactions with other HCPs in the OR and/or the surrounding OR. The audio AR computing system may remove distractions and/or overwhelming sounds (e.g., distracting sounds) from the AR content.

The audio AR computing system may adjust the AR content and insert calm music and/or voice to help the user stay calm, e.g., based on the adjustment indication. For example, the audio AR computing system may adjust the AR content to provide white noise, calm music, and/or music preferred and/or preconfigured by the user. The audio AR computing system may send the adjusted AR content with calming music or white noise to help the user focused on the current step associated with a surgical operation.

In examples, the audio AR computing system may adjust the generated AR content by adjusting an audio AR setting associated with the generated AR content. The audio AR computing system may adjust audio AR setting associated with the generated AR content. In examples, the audio AR computing system may amplify and/or increase volume of the generated AR content. In examples, the audio AR computing system may reduce and/or decrease volume of the generated AR content. In examples, the audio AR computing system may increase a frequency of the generated AR content transmitted to the user or decrease the frequency of the generated AR content transmitted to the user.

The audio AR computing system may adjust the AR content, such as the audio AR setting, based on the adjustment indication. The adjustment indication may be or may include a surgical task indication and/or a task importance indication. The surgical task indication may indicate a surgical task, such as the current surgical task being performed or a pending/upcoming surgical task to be performed. The task importance indication may indicate an importance and/or a criticality of the surgical task.

In examples, the audio AR computing system may amplify and/or increase the volume of the AR content based on the surgical task indication and/or the task importance indication indicating that the surgical task is an important and/or a critical task. The user may listen to the amplified and/or increased volume of the AR content and may be focus.

In examples, the audio AR computing system may reduce and/or decrease the volume of the AR content based on the surgical task indication and/or the task importance indication indicating that the surgical task is a non-important (e.g., less important) and/or a non-critical task. The user may listen to the reduced and/or decreased volume of the AR content and may relax.

The AR content may include multiple audio streams from multiple data sources. In examples, the audio AR computing system may identify an importance of an audio stream to a surgical step. The audio AR computing system may adjust the volume of the audio stream based on the current surgical step and the important of the audio stream to the surgical step. For example, when the audio stream is important to the surgical step, the volume of the audio stream may be increased; when the audio stream is not important to the surgical step, the volume of the audio stream may be decreased.

In examples, the AR content may include multiple audio streams from multiple data sources. For example, the AR content may be or may include multiple audio data from multiple sensing systems. The audio AR computing system may receive audio data from a sensing system in the OR. The audio AR computing system may receive the adjustment indication indicating a user preference setting associated with a surgical operation. For example, the user preference setting may be or may include preferred measurement data for the surgical operation. The audio AR computing system may receive other audio data from other sensing system in the OR. The audio AR computing system may select a preferred audio data. For example, the audio AR computing system may select the preferred audio data between the multiple audio data from the multiple sensing systems indicated in the user preference setting. As described herein, the audio AR computing system may adjust the AR content by reducing (e.g., decreasing) a volume of unselected audio data from the sensing system and/or amplifying (e.g., increasing) a volume of selected audio data from the sensing system. The audio AR computing system may adjust the AR content by increasing the frequency of the selected AR content, and/or decreasing frequency of the unselected AR content. The audio AR computing system may adjust the AR content by blocking the unselected audio data.

In examples, the audio AR computing system may increase the frequency of the generated AR content played for the user based on the surgical task indication and/or the task importance indication. The task importance indication may indicate that the surgical task is an important (e.g., critical) task. In examples, the audio AR computing system may increase the frequency of the generated AR content if an emergency arises (e.g., a measurement data of a patient falls below a threshold level or reaches above a threshold level). For example, if the measurement data associated with the heartbeat of the patient suddenly changes, the audio AR computing system may increase the frequency of notifying the heartbeat measurement data to the user, such as the surgeon. The user may listen to the increased frequency of the heartbeat measurement data and know real-time measurement data.

In examples, the audio AR computing system may decrease the frequency of the AR content transmitted to the user based on the surgical task indication and/or the task importance indication indicating that the surgical task is an important (e.g., critical) task. In examples, the audio AR computing system may decrease the frequency of the AR content transmitted to the user if the emergency passes (e.g., if the measurement data of the patient goes back to normal level). For example, if the measurement data is associated with the heartbeat of the patient and if the heartbeat data goes back to normal (e.g., if an emergency was averted), the audio AR computing system may decrease the frequency of notifying the heartbeat measurement data to the user of the audio AR computing system. As the emergency is averted and/or the patient is stable, the user, such as the surgeon, may listen to and/or focus on other AR content.

In examples, the audio AR computing system may adjust the AR content, such as voice associated with the AR content. For example, the adjustment indication may be or may include a user preference and/or a user setting. Based on the user preference and/or the user setting, the audio AR computing system may alter the voices of the AR content to different voices. For example, different voices may be or may include Morgan Freeman, Denzel Washington, Darth Vader, and/or other voices that the user prefers to listen to.

In examples, the audio AR computing system may adjust the AR content by translating languages. For example, the adjustment indication may indicate that the AR content is in different language, e.g., non-English AR content. The audio AR computing system may translate the AR content in English or the language that the user may understand in real time and may facilitate better communication. In examples, a user, such as a surgeon, may travel to a foreign country and/or may work with HCPs who are not fluent with the language that the user speaks. For example, the surgeon may travel to other locations (e.g., based on the specialty and/or a global program, such as doctors without borders). The surgeon may not be fluent in the local language and may be unable to understand what is being said in the OR and/or ambient conversations. The audio AR computing system may adjust the AR content that is associated with the audio data of the OR and/or audio data in other language and may adjust the AR content by translating the AR content into the language that the user may understand, e.g., in real time.

In examples, the audio AR computing system may adjust the AR content based on an audio source location indication in the adjustment indication. For example, the audio source location indication may indicate an audio source location of the audible data associated with the AR content. The audio AR computing system may adjust the AR content based on the audio source location indication. In example, if the audio source location indication indicates that the audible data originates from outside of an area of interest (e.g., outside of the OR), the audio AR computing system may adjust the AR content by canceling the audible data originating from the outside of the OR. In examples, the audio AR computing system may expect audible data originating from the outside of the OR. For example, the user of the audio AR computing system may expect a phone call from an organ transplant personnel, other surgeons, HCPs in different ORs, experts located in different location (e.g., different countries), a technician from a surgical instrument company, and/or the like. The audio AR computing system may adjust the AR content by allowing the audio data originating from outside of the OR, upon determining that the audio source location associated with audio data is an expected source location.

In examples, the audio AR computing system may adjust the AR content by selecting and/or prioritizing the AR content, e.g., based on the adjustment indication. For example, the AR content may be or may include audible information from one or more computing devices and/or computing systems. The adjustment indication may be or may include a prioritization indication indicating a priority of audible data. If the audio AR computing system determines that the generated AR content is or includes two or more audible data and/or audible information, the audio AR computing system may adjust the AR content by selecting an audible data and/or audible information based on the indicated prioritization information. The audio AR computing system may amplify and/or increase volume of the prioritized audible data. In examples, the audio AR computing system may cancel other audible data. In examples, the audio AR computing system may reduce and/or decrease the volume of the non-priority audible data. The priority of audio data/audible information may be set via a user preference indication indicating a preference for audible data.

In examples, an audio AR computing system may receive an ambient noise level indication.

The ambient noise level indication may indicate an ambient noise level of the operating room. In examples, the audio AR computing system may determine an ambient noise level of an OR. If the audio AR computing system determines that the ambient noise level is below a threshold ambient noise level, the audio AR computing system may determine that a critical surgical task is to be performed or is being performed. In examples, the audio AR computing system may adjust the AR content based on the ambient noise level dropping below a threshold ambient noise level. For example, the audio AR computing system cancel certain audible data in the AR content. For example, the audio AR computing system may provide a quiet and/or calm environment for the user to focus. In examples, the audio AR computing system may send a critical task indication to a surgical computing system. For example, the critical task indication may indicate a critical surgical task is to be performed in the OR (or is being performed). The computing system may send an alert(s) to other HCPs in the OR that an upcoming task involves a critical surgical task.

In examples, an audio AR computing system may send a user input request to a surgical computing system. The audio AR computing system may send the user input request to the surgical computing system before adjusting the AR content. If the user does not provide an input (e.g., confirmation of a suggest AR adjustment) to the user input request, the audio AR computing system may skip adjusting the AR content. In examples, the audio AR computing system determines that the user input is unregistered for a period of time (e.g., a preconfigured time), the audio AR computing system may send a reminder to the user about the user input request. In examples, if the audio AR computing system determines that the user input is unregistered for a period of time, the audio AR computing system may refer to a preconfigured setting (e.g., a default setting). The user may have preselected and/or preconfigured the default setting (e.g., the volume level for the AR content and/or the frequency of receiving the AR content). For example, the user may preconfigure the audio AR computing system to adjust the AR content to the default setting if the audio AR computing system does not register the user input after a preconfigured time (e.g., after 20 seconds) and/or after the audio AR computing system sends a reminder.

The audio AR computing system may receive the user input associated with (e.g., in response to) the user input request. If the audio AR computing system receives the user input, the audio AR computing system may adjust the AR content (e.g., adjust the AR content further) based on the user input. For example, as described herein, based on the user input, the audio AR computing system may further adjust the AR content by amplifying (e.g., increasing) volume of the AR content, reducing (e.g., decreasing) the volume of the AR content, increasing frequency of the AR content, and/or decreasing frequency of the AR content.

An audio AR computing system may adjust generated AR content based on an adjustment indication, e.g., that may be or may include a surgical step indication. For example, the surgical step indication may indicate a current and/or an upcoming surgical step associated with a surgical operation. The audio AR computing system may detect and/or may be aware of the current and/or the upcoming surgical step, e.g., based on the surgical step indication. In examples, the audio AR computing system may adjust the AR content based on a determination (e.g., situational awareness) that audio data of an HCP role that is relevant to the current and/or the upcoming surgical step. As described herein, the audio AR computing system may adjust the AR content by allowing the audio data of the relevant HCP role (e.g., a head nurse) and canceling the audio data associated with other HCP roles (e.g., and/or ambient noise).

Determination of a user or an HCP role is further described in Atty Docket: END9290USNP17 titled ACTIVE RECOGNITION AND PAIRING SENSING SYSTEMS, filed contemporaneously, which is incorporated by reference herein in its entirety. For example, as described herein, an AR computing system may receive user role identification data from one or more sensing systems in an OR. The user role identification data may be or may include information to identify a user role. The surgical computing system may identify a user role for a user in the OR based on the received user role identification data. The user role of a user in the OR may be or may include at least one of a surgeon, a nurse, a patient, a hospital staff, or an HCP.

In examples, the audio AR computing system may receive audio data associated with an HCP role (e.g., a resident) in the OR. The audio AR computing system may receive another audio data associated with another HCP role (e.g., a head nurse) in the OR. The audio AR computing system may determine whether the audio data is relevant to the surgical step indicated in the surgical step indication. For example, the audio AR computing system may determine whether the audio data of the resident and/or the head nurse in the OR is relevant to the surgical step indicated in the surgical step indication. If the audio AR computing system determines that the audio data is relevant to the surgical step, the audio AR computing system may adjust the AR content by allowing the relevant audio data. For example, if the audio AR computing system determines that the audio data of the resident is relevant to the surgical step and the audio data of the head nurse is irrelevant to the surgical step, the audio AR computing system may adjust the AR content by passing through (e.g., allowing) the audio data of the resident and blocking the audio data of the head nurse.

Determination of a user or an HCP role is further described in Atty Docket: END9290USNP17 titled ACTIVE RECOGNITION AND PAIRING SENSING SYSTEMS, filed contemporaneously, which is incorporated by reference herein in its entirety.

For example, an AR computing system may receive user role identification data from one or more sensing systems in an OR. The user role identification data may be or may include information to identify a user role. The surgical computing system may identify a user role for a user in the OR based on the received user role identification data. The user role of a user in the OR may be or may include at least one of a surgeon, a nurse, a patient, a hospital staff, or an HCP. Based on the identified user role, the surgical computing system may generate surgical aid information for the user in the OR. The surgical aid information may be or may include information associated with a surgical operation that is relevant to the identified user role. The AR computing system may transmit relevant information to the identified user, e.g., via AR content as described herein.

The user role identification data may be or may include one or more of the following: a proximity of a user to a surgical instrument, locations and/or location tracking information of the users in the OR, interactions between the user and at least one HCP, one or more surgical procedural activities, or visual data of the user in the OR. For example, the sensing system may be worn by the user such as a surgeon. The sensing system may monitor and/or store information about the proximity of the sensing system to a surgical instrument. The sensing system may store location tracking information of the surgeon during a surgical procedure. The sensing system may detect and/or store a surgical procedural activity of the surgeon. The sensing system may send such user role identification data to the surgical computing system.

For example, as described herein, the AR computing system may generate AR content for a user based on the identified user role. Different AR content may be generated for different users based on their respective user roles identified via the sensing systems. The AR content may be or may include instructions on how to use a surgical instrument and/or an operation manual of the surgical instrument associated with the identified user role. The surgical computing system may send the generated AR content to the identified user. For example, the surgical computing system may send the AR content to an AR device associated with the user.

In examples, the audio AR computing system may adjust the AR content by amplifying (e.g., increasing) volume of the audio data associated with the HCP role that is relevant to the surgical step, reducing (e.g., decreasing) the volume of the audio data associated with the HCP role that is irrelevant to the surgical step, increasing frequency of the audio data associated with the HCP role that is relevant to the surgical step, and/or decreasing frequency of the audio data associated with the HCP role that is irrelevant to the surgical step.

As described herein, the audio AR computing system may adjust the AR content based on awareness of an OR, e.g., based on an ambient noise level indication. If the audio AR computing system determines that the ambient noise level indication is below a threshold noise level, the audio AR computing system may determine that a current and/or an upcoming surgical step (e.g., task) is a critical step (e.g., task). The audio AR computing system may adjust the AR content by allowing the relevant audible data associated with the critical surgical task and/or block other audible data associated with non-critical surgical task (e.g., ambient noise). The audio AR computing system may adjust the AR content by amplifying (e.g., increasing) volume of the audio data associated with the critical surgical task, increasing frequency of the audio data associated with the critical surgical task, reducing (e.g., decreasing) volume of the audio data associated with the non-critical surgical task, and/or decreasing frequency of the audio data associated with the non-critical surgical task.

The audio AR computing system may adjust the AR content-based awareness of a user condition. For example, the audio AR computing system may determine that a stress level of a user has increased, e.g., based on measurement data associated with the user. Based on the increased stress level, the audio AR computing system may derive that a current and/or an upcoming surgical task is a critical task. If the audio AR computing system determines that the current and/or the upcoming surgical task is the critical task, the audio AR computing system may adjust the AR content. For example, as described herein, the audio AR computing system may adjust the AR content by allowing the relevant audible data associated with the critical surgical task to pass through and/or block other audible data associated with non-critical surgical task (e.g., ambient noise). Upon detecting the user's increased stress level, the audio AR computing system may adjust the AR content by amplifying (e.g., increasing) volume of the audio data associated with the critical surgical task, increasing frequency of the audio data associated with the critical surgical task, reducing (e.g., decreasing) volume of the audio data associated with the non-critical surgical task, and/or decreasing frequency of the audio data associated with the non-critical surgical task. Upon detecting the user's increased stress level, the audio AR computing system may adjust the AR content by inserting calming audio.

Determination of a stress level is further described in Atty Docket: END9290USNP2 titled ADAPTABLE SURGICAL INSTRUMENT CONTROL, filed contemporaneously, which is incorporated by reference herein in its entirety.

For example, the computing system may receive measurement data from one of the sensing systems associated with the users in the operating room (e.g., sensing system associated with a surgeon). The computing system may also receive measurement data from one of the sensing systems associated with the users in the operating room indicating higher stress level of the users. For example, higher stress level may be indicated by change in the users' heart rate from a base value. The computing system may derive this inference by cross-referencing the receipt of data from the corresponding sensing systems. The computing system may send surgical aid information to the identified user as described herein.

The audio AR computing system may adjust the AR content based on awareness of a user condition. For example, the audio AR computing system may determine that a fatigue level of a user (e.g., the user wearing the audio AR computing system) has increased, e.g., based on measurement data associated with the user. Based on the increased fatigue level, the audio AR computing system may be aware and/or determine that the user may need to be focused. If the audio AR computing system determines that the user needs to be focused, the audio AR computing system may adjust the AR content. For example, as described herein, the audio AR computing system may adjust the AR content by allowing the relevant audible data associated with a current surgical task and/or block other audible data that are not associated with the current surgical task (e.g., ambient noise). The audio AR computing system may adjust the AR content by one or more of amplifying (e.g., increasing) volume of the audio data associated with the current surgical task and/or increase frequency of the audio data associated with the current surgical task. The audible AR component may adjust the AR content by one or more of reducing (e.g., decreasing) volume of the audio data that is not associated with the current surgical task and/or decrease frequency of the audio data that is not associated with the current surgical task.

Determination of a fatigue level is further described in Atty Docket: END9290USNP2 titled ADAPTABLE SURGICAL INSTRUMENT CONTROL, filed contemporaneously, which is incorporated by reference herein in its entirety.

For example, the AR computing system may receive measurement data from one of the sensing systems associated with the users in the OR (e.g., sensing system associated with a surgeon). The measurement data may indicate the users, such as a surgeon, make too large of a change in input, which may be referred to as over-correction, for a perceived mistake. The AR computing system may interpret repeated correction, over-correction, or oscillating reaction as an indicator of fatigue and/or elevated fatigue level associated with the identified user.

The AR computing system may be configured to analyze usage data and/or measurement data to determine whether a user working in the OR is experiencing fatigue and, if so, to modify operation of the surgical instrument and/or to provide notifications associated with the fatigue levels. For example, the AR computing system may monitor user inputs to a surgical instrument (e.g., from the surgical instrument and/or from sensing systems). The user inputs to the surgical instrument may include inputs that result in shaking of the surgical instrument. Shaking, whether done intentionally or otherwise, may be detected by one or more sensing systems (e.g., acceleration sensors) which provide data regarding the movement and orientation of the surgical instrument. The detected data may indicate magnitude and frequency of any tremors. The surgical instrument may generate usage data associated with the monitored user inputs. The usage data may indicate the inputs to the surgical instrument, e.g., including movements of all or a portion of the surgical instrument including shaking. The usage data may be communicated to the AR computing system.

Data may be collected from sensing systems that may be applied to the users of the surgical instrument as well as other HCPs who may assist in the OR. Accelerometers may be applied to the users' hands, wrists, and/or arms. Accelerometers may also be applied to users' torsos to gather data associated with the body movements including swaying and body tremors. The accelerometers may generate data regarding motion and orientation of the users' hands and/or arms. The data may indicate magnitude and frequency of movements including shaking. Sensing systems (e.g., that may be or may include accelerometers) may collect biomarker data from the users including data associated with heartbeat, respiration, temperature, etc. The sensing systems may collect data associated with the hydration/dehydration of the corresponding users operating the surgical instrument as well as the other users assisting in the OR. The gathered data may be communicated to the AR computing system.

The AR computing system may receive usage data from the surgical instrument and may receive sensor data from the sensing systems corresponding to the users in the OR. The AR computing system may identify and/or store the received data in association with time stamp data indicating time the data was collected corresponding to the user.

The AR computing system may determine, based on the received usage data and/or sensor data, fatigue levels for the users operating the surgical instrument and assisting in the OR. The AR computing system may determine, based on the received usage data and sensor data, time periods associated with the surgical procedure. The AR computing system may determine, for each users, values associated with time in the OR, time spent standing in the OR, time spent physically exerting themselves. The AR computing system may determine fatigue levels for the users based on the time spent in surgery.

The AR computing system may determine, based on the received usage data and/or sensor data, physical indications of fatigue. The AR computing system may determine, if the received data indicates a user is swaying or unsteady, that the user is fatigued. The AR computing system may determine, if the received data indicates tremors are exhibited by a user, that the user is fatigued.

The AR computing system may determine, based on the received usage data and sensor data, values associated with hydration/dehydration of the users in the OR. Dehydration may impact energy levels and make a person feel tired and fatigued. Less body fluid tends to increase heart rate. The AR computing system may analyze heartbeat data in the context of hydration levels and differentiate between stress and other heart elevation events from hydration. The AR computing system may employ a baseline measure to differentiate acute events from ongoing chronic events and to differentiate between fatigue and dehydration associated with each users in the OR.

The AR computing system may calculate a weighted measure of fatigue for the user operating the surgical instrument as well as others in the OR. The weighted measure of fatigue may be based on cumulative cooperative events and contributions. For example, the weighted measure of fatigue may be based on the intensity of stress experienced by a user and the force exerted by the user over time in controlling an actuator such as closure trigger over time.

If the AR computing system determines that the users have experienced fatigue, the AR computing system may determine to communicate control features to the surgical instrument, to other AR computing systems associated with HCPs in the OR, and/or the AR computing system of the user whose fatigue level has been elevated. The communicated control features may be or may include fatigue control or accommodation and adjustment to compensate for fatigue. The control feature to perform fatigue control may indicate to reduce the force required to implement an action. For example, the control feature may indicate to reduce the force needed to be applied to a closure trigger to activate clamping jaws of a surgical instrument. The control feature may indicate to increase the sensitivity of the closure trigger. The control features may indicate to increase delay or wait time responsive to user inputs. The control features may indicate to slow activation and provide additional time before acting.

If the computing system determines the users have experienced fatigue, the AR computing system may also determine to communicate control features to provide notifications regarding the fatigue. The AR computing system may determine that notifications regarding fatigue may be provided by the surgical instrument to the user. The AR computing system may determine that the notifications may provide more steps-for-use to the operator. The AR computing system may also determine that notifications regarding fatigue levels may be made to persons in the OR other than the HCP manning the instrument. Such notifications may be displayed on display systems in or near the OR.

The AR computing system may communicate an indication of a control features associated with fatigue control. The control features may be communicated to the surgical instrument, the AR computing system, and/or may also be communicated to other systems in the OR such as display which may be employed to provide notifications.

The surgical instrument and display may receive the indication of control features indicating to implement fatigue control and provide notifications. The surgical instrument may determine to operate consistent with the indication of fatigue control. The instrument may reduce the force required to activate and/or operate closure trigger. The surgical instrument may increase the delay or wait time between requesting an action, e.g., applying force to the closure trigger, and implementing the corresponding action, e.g., closing the jaws. The surgical instrument may slow activation in response to inputs and thereby provide more time for the operator to position the surgical instrument.

If the control features indicate to provide notifications, the surgical instrument may provide physical tactile feedback as well as visual feedback. The display may also provide visual feedback regarding fatigue. The notifications may provide steps-for-use to minimize overlooking of details.

In examples, if an audio AR computing system receives critical audible information associated with a patient (e.g., sudden changes in the condition of the patient), the audio AR computing system may allow the critical audible information associated with the patient condition to be transmitted. For example, the audio AR computing system may exclude the critical audible information associated with the patient condition from being adjusted (e.g., canceled).

A computing system, such as an audio AR computing system and/or a visual AR computing system, may interpolate data, such as AR data and/or AR content, to be overlaid with an augmented array. In example, an audio AR computing system may interpolate audible AR content and the user of the audio AR computing system may listen to overall changes to the AR content (e.g., patient's measurement data associated with conditions of the patient). In examples, a visual AR computing system may interpolate visual AR content and the user of the visual AR computing system may view overall changes to the AR content (e.g., the measurement data associated with conditions of the patient).

In examples, the audio AR computing system may provide audible information associated with gradients of a marker over a patient (e.g., a body of the patient) or gradients over time to improved condition for the patient. The user of the audio AR computing system may understand the condition of the patient through the audio AR computing system and/or audible AR content.

In examples, the visual AR computing system may provide visual information associated with gradients of a marker over a patient (e.g., a body of the patient) or gradients over time to improved condition for the patient. The user of the visual AR computing system may understand the condition of the patient through the visual AR computing system and/or visual AR content.

The audible AR content and/or visual AR content may be or may include one or more information from a camera in an OR, an image of the patient's body (e.g., MRI, MRA, and/or the like), a camera inside the body of the patient, and/or the like.

The audible AR content and/or visual AR content may be or may include one or more information associated with temperatures of a patient, such as core temperature of the patient and/or peripheral temperatures of the patient. The audible AR content and/or visual AR content may be or may include gradient of the temperature that are plotted onto the body the patient. The audio AR computing system may provide an audible AR content and the user, such as a surgeon, may listen to the audible AR content (e.g., temperature information of the patient and/or the gradient temperature information of the patient). The visual AR computing system may provide a visual AR content, such as an overlay gradient temperature information of the patient. The user of the visual AR computing system may look at the visual AR content and/or monitor variations in patterns for the temperature of the patient.

In examples, an AR computing system (e.g., an audio AR computing system and/or a visual AR computing system) may receive AR contents, such as measurement data of a patient, from one or more other computing systems and/or computing devices. The AR computing system may receive and/or gather the measurement data of the patient and generate an AR content associated with the measurement data of the patient. The AR computing system may transmit the audible information associated with the measurement data of the patient. The AR computing system may show the visual information associated with the measurement data of the patient. The AR content may be or may include gradient information of the patient and/or changes in the measurement data of the patient over time.

An AR computing system (e.g., an audio AR computing system and/or a visual AR computing system) may provide AR content that may be or may include measurement data. In examples, an audio AR computing system may provide audible AR content for measurement data of a patient, e.g., locally to a user who is wearing the audio AR computing system. In examples, a visual AR computing system may provide visual AR content for measurement data of a patient, e.g., locally to a user who is wearing the visual AR computing system. The AR content may be information overlay of the measurement data of the patient. The AR content may provide data depth to the user, e.g., via the AR overlays.

An AR computing system may receive one or more measurement data from one or more sensing systems. For example, the AR computing system may receive one or more measurement data from one or more sensing systems located in an OR. The measurement data may be or may include measurement data of a patient and/or a user, such as a surgeon.

In examples, the audio AR computing system may generate an audible AR content based on the measurement data. As described herein, the audio AR computing system may overlay audible information of the measurement data and may generate and/or adjust the AR content. For example, the audio AR computing system may overlay audible AR content associated with the monitored and/or real-time measurement data of the patient and/or the user (e.g., such as the surgeon). In examples, the audio AR computing system may transmit the AR content using an audio output associated with the audio AR computing system and provide the AR content locally to a user. In examples, the audio AR computing system may share the audible AR content to a speaker and/or an audio output connected to an OR (e.g., to broadcast) and/or other audio AR computing systems associated with other HCPs in the OR.

In examples, the visual AR computing system may generate a visual AR content based on the measurement data. As described herein, the visual AR computing system may overlay visual information of the one or more measurement data and may generate and/or adjust the AR content. For example, the visual AR computing system may overlay the visual AR content associated with the monitored and/or real-time measurement data of the patient and/or the user (e.g., such as the surgeon). In examples, the visual AR computing system may transmit the AR content using a display associated with the visual AR computing system and provide the AR content locally to a user. In examples, the visual AR computing system may share the visual AR content to a display and/or a monitor connected to an OR (e.g., to broadcast) and/or other visual AR computing systems of other users (e.g., HCPs) in the OR.

In examples, as described herein, the audio AR computing system and/or the visual AR computing system may request a user input prior to sharing the information. If the audio AR computing system and/or the visual AR computing system does not receive the user input, the audio AR computing system and/or the visual AR computing system may send a reminder user input request and/or defer to a preconfigured setting (e.g., a default setting). In examples, the preconfigured setting may be or may include skip sharing the information to other HCPs and/or skip broadcasting to the OR. In examples, the preconfigured setting may be or may include sharing the measurement data to the HCPs and/or broadcast to the OR.

In examples, if the audio AR computing system and/or the visual AR computing system does not receive the user input (e.g., from the primary user, such as a surgeon), the audio AR computing system and/or the visual AR computing system may send a user input request to one or more other HCPs in the operating room. For example, if the audio AR computing system and/or the visual AR computing system does not receive an input from the surgeon, the audio AR computing system and/or the visual AR computing system may send the user input request to a head nurse. The head nurse may provide the user input. The surgeon may work with the list of HCPs, such as the head nurse. The head nurse may remember the surgeon's preference and/or prior instruction from the surgeon. Based on the instruction and/or known preference of the surgeon, the other HCPs, such as the head nurse, may provide the user input for the surgeon. The audio AR computing system and/or the visual AR computing system may share the AR content locally and/or broadcast the AR content in the OR and/or to other HCPs in the OR.

In examples, the audio AR computing system and/or the visual AR computing system may receive measurement data from one or more sensing systems and/or from a surgical computing system. As described herein, the audio AR computing system and/or the visual AR computing system may adjust the AR content (e.g., audible AR content or visual AR content) The AR device may display the received data (e.g., wearable data). In examples, the audio AR computing system and/or the visual AR computing system may highlight a particular measurement data (e.g., an important information such as blood pressure and/or heart monitor information of a patient).

In examples, the audio AR computing system may adjust the AR content and may amplify (e.g., increase volume) of the audible information associated with the particular measurement data that is relevant and/or important to a current surgical procedure. The audio AR computing system may adjust the AR based on awareness of the surgical procedure, atmosphere of the OR, and/or interactions between HCPs as described herein. The audio AR computing system may adjust the AR content based on an adjustment indication received from a surgical computing system that indicates important and/or relevant measurement data associated with a current surgical task.

In examples, the visual AR computing system may adjust the AR content and may increase the resolution on the visual information associated with the particular measurement data that is relevant and/or important to a current surgical procedure. For example, the visual AR computing system may provide a higher resolution on the relevant and/or the important measurement data for the current surgical procedure. The visual AR computing system may adjust the AR based on the awareness of the surgical procedure, atmosphere of the OR, and/or interactions between HCPs as described herein. The visual AR computing system may adjust the AR content based on an adjustment indication received from a surgical computing system that indicates important and/or relevant measurement data associated with a current surgical task.

The audio AR computing system and/or the visual AR computing system may provide the measurement data simultaneously and/or continuously. For example, the audio AR computing system may adjust the AR content that may be or may include measurement data and continuously provide the audible information associated with the measurement data. In examples, the audio AR computing system may provide the audible information in the same tone. In examples, the audio AR computing system may adjust the AR content and may highlight important and/or relevant measurement data in a different tone, volume, and/or voice.

The visual AR computing system may adjust the AR content and may display one or more (e.g., all) measurement data from the sensing systems simultaneously. In examples, the visual AR computing system may display the measurement data in the same resolution. In examples, the visual AR computing system may adjust the AR content and may provide a higher resolution and/or different resolution to highlight the important and/or the relevant measurement data. In examples, the visual AR computing system may adjust AR content and may enlarge the important and/or the relevant measurement data and/or truncate other measurement data.

In examples, the AR computing system may select audible information and/or visual information to adjust AR content based on a current step of the operation. For example, as described herein, the AR computing system may be situationally aware of the current step and/or task of the operation. The AR computing system may select relevant AR information for adjusting the AR content. The AR may send the unselected audible AR information and/or the visual AR information to the HCPs. For example, a surgeon may receive the AR content with the relevant AR information that is associated with the current step of the surgical operation. In examples, other HCPs in the OR may receive the same information. In examples, other HCPs in the OR may receive other AR information and may monitor the information.

An AR computing system may send a request to one or more sensing systems and/or a surgical computing system. The request may be or may include additional measurement data and/or updated data. For example, based on the current step and/or task, the AR computing system may prioritize AR content and may skip receiving updates on the AR information. In examples, during an emergency, the AR computing system may skip receiving heartbeat tracing information, electrocardiogram (EKG) information, and/or heart rate variability information of the patient. After the emergency, the AR computing system may resume receiving such information. For example, the AR system may send an update request for the skipped measurement data to one or more sensing systems and/or to the surgical computing system. The AR computing system may receive the updated and/or monitored measurement data.

In examples, the AR computing system may receive one or more measurement data from corresponding one or more sensing systems that are associated with a patient. The sensing system may have been tracking measurement data associated with the patient. For example, the measurement data may be or may include heartbeat tracing information, EKG information, and/or heart rate variability information of the patient. The sensing systems may have measurement data of the patient over a period of time (e.g., prior to the surgery) and may show history of the measurement data. The measurement data may be and/or may include real-time measurement data of the patient.

A user of the AR computing system may preconfigure (e.g., preset) AR settings associated with receiving audible AR information and/or visual AR information in an AR content. For example, the user may configure the frequency of receiving the audible AR information and/or the visual AR information (e.g., every 5 seconds or every minute). The user may configure the volume and/or the resolution of the audible AR information and/or the visual AR information. The user may configure the AR setting associated with the audible AR information and/or the visual AR information prior to the surgery, based on history data of the user preference, and/or during the surgery.

Measurement data from a sensing system may be used for risk assessment and may be applied to a surgical procedure (e.g., compatible surgical procedure). A computing system may use data to assess risk for a surgical procedure. The computing system may use the data to inform go/no go surgical decision.

A sensing system may gather measurement data. For example, a sensing system may gather measurement data (e.g., sensor data) prior to a surgical procedure. The sensing system may monitor one or more variables (e.g., specific variables) and may provide frequency updates to an HCP, such as a surgeon, prior to surgery. The measurement data may help inform the surgeon if acceptable conditions are in place prior to a scheduled surgery. In examples, international normalized ration (INR) may be a metric used to assess coagulation of blood, e.g., in a patient on coumadin (e.g., fairly common anti-coagulant). Elevated levels (e.g., elevated levels of measurement data) may be common for a patient on anti-coagulant therapy. The elevated levels may be associated with bleeding complications in elective and/or emergent surgical procedures.

A computing system may monitor an absolute value of INR and/or any change in the value of INR (e.g., trend) prior to a surgery. The absolute INR value, the INR value, and/or a trend of INR value may indicate readiness of a patient for a surgery. A guideline (e.g., a surgical guideline) may recommend stopping coumadin 5-6 days prior to the surgery and/or administration of reversal treatment approximately 6 hours prior to the surgery. If the patient is in the hospital, the patient's vitals and/or other information may be easily tracked. If the patient is not in the hospital, the patient's vitals and/or other information may not be easily tracked. If the patient is not in the hospital, the patient's vitals and/or other information may be tracked the day prior or day of the surgery. Having patient's vitals and/or other information the day prior to or the day of the surgery may lead to planning challenges and/or increased bleeding risk in the OR (e.g., and/or increased procedure costs).

One or more computing systems and one or more sensing systems may communicate and share measurement data (e.g., lab testing) and provide an overall analysis (e.g., improved overall analysis). A computing device may interact with other data sources (e.g., a hub and/or a sensing system) and/or may impact patient pre-operative or post-operative care.

Combination of multiple data sources may provide a patient care directive (e.g., an optimal patient care directive). For example, pre op care change of change in diet and/or change in medications for renal function may be implemented. For example, post op care (e.g., particular diet modifications and/or stratified risk for dialysis) may be implemented. In examples, low serum albumin levels may be associated with poor surgical outcomes (e.g., increased morbidity and/or mortality). The low serum albumin levels may or may not be related to nutrition. In examples, coupling serum albumin measurements with change in weight (e.g., measurement data from a wireless scale) may help control for low albumin from malnutrition, low albumin from kidney disease, and/or other pathologic condition. In examples, bioimpedance analysis may be combined with measurement data from a scale. The combined bioimpedance analysis and the measurement data from the scale may help elucidate water changes in body weight on a patient.

A computing system may monitor preconditioning of a patient. For example, a computing system may monitor and/or look for readiness thresholds from the monitored preconditioning of the patient. Preconditioning of a patient may prepare the patient for a surgery and/or may monitor for the patient to achieve a thresholds set by an HCP, such as a surgeon.

A computing system may use pre-operative patient monitoring data and/or conditioning to train a body based on surgery time. A sensing system may collect measurement data. For example, the measurement data may include: heart rate, respiration rate, temp, sleep, mental state, and/or the like. The computing system may determine when a patient should have a surgery performed based on the measurement data.

In (e.g., addition to and/or alternative to) examples, the computing system may use the measurement to train the body and/or subconscious mind to be conditioned for a certain time. Based on the monitored data, a computing system may set one or more triggers at certain times to lower anxiety, reduce heart rate and breathing rate to minimize inflammation, provide indication to a user to rest, and/or the like. The computing system may tier the triggers to a certain time. For example, the computing system may tier the triggers to a certain time, and the mind and body was conditioned and could be more relaxed at the time of surgery.

In (e.g., addition to and/or alternative to) examples, the computing system may utilize the measurement data and/or based on one or more triggers. The one or more triggers may pop up a video on the patient's device, such as a phone, to watch. The patient may watch the video to relax, lower pulse rate, and/or alter breathing. The patient may listen to an audio recording, e.g., deliberately altering the frequency of patient's brainwaves. For example, brainwaves of a patient may fall into a specific frequency depending on what the patient is doing at a given time. The brainwaves may be gamma if the patient is engaged in certain motor functions. The brainwaves may be beta if the patient is fully conscious and/or actively concentrating. The brainwaves may be alpha if the patient is relaxed. The brainwaves may be theta if the patient is drowsy and/or lightly sleeping. The brainwaves may be delta if the patient is in deep sleep.

Binaural beats may result if two tones are played at differing frequencies. The binaural beats may trigger brainwaves of the patient to follow a different pattern. For example, if a computing device (e.g., using the measurement data) wants to shift a patient's state from stressed to relaxed, the computing device may play audio and the patient may listen to an audio that triggers the alpha state.

An audio program may help reprogram subconscious mind of a patient, e.g., by creating a more receptive forum for installing positive messages. A subconscious mind may be more receptive to new information if a patient/body is relaxed, such as in the alpha or theta states.

Using a brain entrainment audio program and/or affirmations or visualization may be a powerful combination. Subconscious mind of a patient may let down its defenses and may easily absorb a message that an HCP and/or a computing device may wish to program in.

If more than one HCP is involved and the HCPs are one or more sites communication of data between HCPs, coordination and/or treatments between HCPs may be adjusted (e.g., improved).

A computing device (e.g., a wearable device) may provide a reminder(s) to a user. For example, a computing device may provide a reminder to a user of information that a HCP gave, e.g., at the time of discharged. If the reminder does not help resolve a confusion, the computing device may link to a mobile phone, WIFI, and/or other network and allow the HCP to interact with the user in real-time. The reminder may act as a reminder and a means to clear up things that a user needs to improve compliance and/or recovery. In examples, a reminder may be or may include an exercise(s) that is supposed to be done daily and/or a medication(s) that should be taken a certain time. The computing device may remind the user and/or detect that the user is engaging in the recommended exercise and/or taking the medicine. If the computing device does not detect an event and/or an underlying measurement data (e.g., biomarker) indicates a lack of improvement, a computing system may be used to understand if the user is doing the exercise correctly and/or taking the medicine on time. The computing system may notify the HCP and/or the computing system may help the user to remember to do the activities.

After a surgery, a surgeon may provide a primary care physician, physiotherapist, and/or other HCPS information on mobility restrictions and/or exercises that are needed. Other HCPs may modify the medications that the user is taking (e.g., temporarily or depending on measured parameters). Other HCPs may monitor and/or ensure compliance to a pre-surgery and/or post-surgery regiment, such as eating, resting, etc. Other HCPs may have one or more measurement data (e.g., biomarker) thresholds that now hold a higher importance and/or that may trigger an intervention if the measurement data (e.g., the biomarker) does not change over time as expected.

One or more supporting HCPs may record progress and/or compliance of the user. The primary surgeon may have the progress and/or compliance data available when the surgeon review recovery with the patient.

A computing system may include an antenna (e.g., a flexible antenna) and may isolate detection and a communication system. In examples, a computing system may use signal intensity, noise, and/or directional antennas to selectively engage one or more computing devices if a number of computing devices in roman operating room exceeds a threshold number. In examples, a computing device (e.g., a wearable computing device and/or an environmental computing device) may indicate compatibility and/or adjust signal output to be compatible with an unknown computing system. A computing device may move through a range of viable frequencies and/or communication modalities to determine if the computing device may pair to a computing system that the computing device detects.

One or more computing systems and/or other computing devices may communicate with one another. In examples, a computing system may communicate with one or more computing devices (e.g., wearable computing devices). In examples, one or more digital devices may exist. A computing system may detect a surgeon by physical actions and/or automated setup. In example, a computing system may setup one or more instrument operational parameters, e.g., based on the detection of a technique used by the surgeon.

In example, a computing system may detect a user, such as a surgeon in an operating room (OR), based on a physical action by the user. For example, the surgeon may be wearing one or more computing devices, such as a wearable, that may communicate with the computing system. The computing system, based on the information from the one or more computing devices, may determine what action the user is performing. For example, a surgeon may wear a computing device (e.g., a wearable) on his/her wrist. The computing device may detect the surgeon holding an instrument, such as a surgical staple gun. The computing system may receive the information, from the computing device, that the surgeon is holding a surgical staple gun. The computing system may determine one or more steps that the surgeon may take.

A computing system may hybridize one or more static imaging techniques with continuous data monitoring (e.g., from one or more measurement data).

Telemedicine may be interconnected with a computing system. In example, telemedicine appointment scheduling may be based on an intra-operative event. For example, based on intra-op measured data (e.g., parameters), one or more relevant telemedicine providers may be queued and/or booked in the computing system for regular follow-ups.

Single or combination of intraoperative computing devices (e.g., sensors) may flag a patient if the measurement data (e.g., monitored measurement data and/or variables) fall outside of desired values. If the computing devices flag based on the measurement data, an HCP, such as a surgeon, may be alerted, e.g., after a case that telemedicine follow-up is needed in a given specialty. In examples, the telemedicine may be alerted (e.g., automatically) to setup relevant follow-ups.

In example, a computing system and/or a computing device may monitor measurement data, such as serum albumin. If the computing device detects drop in serum albumin (e.g., below a preconfigured threshold serum albumin level), the computing device may prompt a need for scheduled nutritionist intervention post-op. The updates may be conditioned on one or more suitable criterion and/or set of criteria. In examples, an update may be conditioned on one or more hardware capabilities of a computing system, such as processing capability, bandwidth, resolution, and/or the like. In examples, the update may be conditioned on one or more software aspects, such as a purchase of certain software code. In examples, the update may be conditioned on a purchased service tier. The service tier may represent a feature and/or a set of features that the user is entitled to use in connection with the computer-implemented interactive surgical system. The service tier may be determined by a license code, an e-commerce server authentication interaction, a hardware key, a username/password combination, a biometric authentication interaction, a public/private key exchange interaction, and/or the like.

1. An audio augmented reality (AR) computing system comprising:
a processor configured to:
receive audio data from a sensing system in an operating room, the audio data comprising measurement data associated with a user;
generate AR content based on the received audio data;
obtain an adjustment indication indicating adjustment information for the AR content; and
based on the received adjustment indication, adjust the generated AR content.

2. The audio AR computing system of claim 1, wherein the adjustment indication is received from a surgical computing system and comprises at least one of: a surgical task indication indicating a surgical task being performed or to be performed, a task importance indication indicating an importance of the surgical task, an audio insertion indication indicating a calming audio insert, an audio translation indication indicating an audio translation of the audio data, or an audio source location indication indicating an audio source location of the audio data.

3. The audio AR computing system of claim 1, wherein the adjustment indication comprises an importance of a surgical step, and the processor is further configured to:
identify an audio AR setting associated with the importance of the surgical step; and
adjust the AR content in accordance with the audio AR setting.

4. The audio AR computing system of claim 1, wherein the adjustment indication comprises audio information for a critical surgical step, and wherein to adjust the generated AR content, the processor is configured to:
silence the audio data from the sensing system; and
amplify an audio associated with the audio information for the critical surgical step.

5. The audio AR computing system of claim 4, wherein the audio information for the critical surgical step further comprises at least one of an increase frequency indication or a decrease frequency indication, and the processor is configured to:
based on the audio information comprising the increase frequency indication, send an increase frequency request to the sensing system, the increase frequency request comprising a request to increase a frequency of sending the audio data from the sensing system; or
based on the audio information comprising the decrease frequency indication, send a decrease frequency request to the sensing system, the decrease frequency request comprising a request to decrease a frequency of sending the audio data from the sensing system.

6. The audio AR computing system of claim 1, wherein the adjustment indication comprises a surgical task indication indicating a surgical task being performed or to be performed, and the processor is configured to:
identify a relevance of the audio data from the sensing system to the surgical task indicated in the surgical task indication; and
determine whether to block the audio data from the sensing system based on the identified relevance to the surgical task indicated in the surgical task indication, wherein the audio data from the sensing system is blocked on a condition that the audio data from the sensing system is relevant to the surgical task indicated in the surgical task indication.

7. The audio AR computing system of claim 1, wherein the processor is configured to:
receive an ambient noise level indication indicating an ambient noise level of the operating room; and
on a condition that the received ambient noise level is below a threshold ambient noise level, send a critical task indication to a surgical computing system, the critical task indication indicating a critical surgical task is to be performed.

8. The audio AR computing system of claim 1, wherein prior to adjusting the generated AR content, the processor is configured to:
send a user input request to a surgical computing system, the user input request requesting a user input; and
receive the user input associated with the user input request, wherein the AR content is adjusted further based on the user input.

9. The audio AR computing system of claim 1, wherein the adjustment indication comprises a surgical task indication indicating a surgical task that is being performed or that is to be performed, the processor is configured to:
identify an audio AR setting associated with the surgical task; and
adjust the AR content in accordance with the identified audio AR setting.

10. The audio AR computing system of claim 1, wherein the audio data comprises a first audio data, the sensing system comprises a first sensing system, the adjustment indication comprises a user preference setting associated with a surgical operation, wherein the processor is configured to:
receive a second audio data from a second sensing system in the operating room;
based on the user preference setting, select a preferred audio data between the first audio data from the first sensing system and the second audio data from the second sensing system; and
adjust the AR content by increasing a volume of the selected preferred audio data.

11. The audio AR computing system of claim 1, wherein the audio data comprises a first audio data, the sensing system comprises a first sensing system, the adjustment indication comprises a user preference setting associated with a surgical operation, wherein the processor is configured to:
receive a second audio data from a second sensing system in the operating room;
based on the user preference setting, select a preferred audio data between the first audio data from the first sensing system and the second audio data from the second sensing system; and
adjust the AR content by reducing a volume of the first audio data from the first sensing system, on a condition that the second audio data from the second sensing system is selected as the preferred audio data.

12. The audio AR computing system of claim 1, wherein the audio data comprises a first audio data, the sensing system comprises a first sensing system, the adjustment indication comprises a user preference setting associated with a surgical operation, wherein the processor is configured to:
receive a second audio data from a second sensing system in the operating room;
on a condition that the first audio data is preferred based on the user preference setting, perform at least one of:
sending a first increase frequency request to the first sensing system to increase a frequency of sending the first audio data from the first sensing system, or
sending a first decrease frequency request to the second sensing system to decrease a frequency of sending the second audio data from the second sensing system; and
on a condition that the second audio data is preferred based on the user preference setting, perform at least one of:
sending a second increase frequency request to the second sensing system to increase a frequency of sending the second audio data from the second sensing system, or
sending a second decrease frequency request to the first sensing system to decrease a frequency of sending the first audio data from the first sensing system.

13. The audio AR computing system of claim 1, wherein the adjustment indication comprises a surgical step indication indicating a surgical step, and the processor is configured to:
receive a first audio data associated with a first healthcare processional (HCP) role in the operating room;
receive a second audio data associated with a second HCP role in the operating room;
determine whether the first audio data associated with the first HCP is relevant to the surgical step indicated in the surgical step indication and whether the second audio data associated with the first HCP is relevant to the surgical step indicated in the surgical step indication; and
upon determining that the first audio data associated with the first HCP is relevant to the surgical step and that the second audio data associated with the first HCP is not relevant to the surgical step indicated in the surgical step indication, adjust the AR content by allowing the first audio data associated with the first HCP to pass through and blocking the second audio data from the second HCP.

14. A method comprising:
receiving an audio data from a sensing system in an operating room, the audio data comprising measurement data associated with a user;
generating augmented reality (AR) content based on the received audio data;
obtaining an adjustment indication indicating adjustment information for the AR content; and
based on the received adjustment indication, adjusting the generated AR content.

15. The method of claim 14, wherein the adjustment indication is received from a surgical computing system and comprises at least one of: a surgical task indication indicating a surgical task being performed or to be performed, a task importance indication indicating an importance of the surgical task, an audio insertion indication indicating a calming audio insert, an audio translation indication indicating an audio translation of the audio data, or an audio source location indication indicating an audio source location of the audio data.

16. The method of claim 14, wherein the adjustment indication comprises an importance of a surgical step, and the method comprises:
identifying an audio AR setting associated with the importance of the surgical step; and
adjusting the AR content in accordance with the audio AR setting.

17. The method of claim 14, wherein the adjustment indication comprises audio information for a critical surgical step, and wherein to adjust the generated AR content, the method comprises:
silencing the audio data from the sensing system; and
amplifying an audio associated with the audio information for the critical surgical step.

18. The method of claim 17, wherein the audio information for the critical surgical step further comprises at least one of an increase frequency indication or a decrease frequency indication, and the method comprises:
based on the audio information comprising the increase frequency indication, sending an increase frequency request to the sensing system, the increase frequency request comprising a request to increase a frequency of sending the audio data from the sensing system; or
based on the audio information comprising the decrease frequency indication, sending a decrease frequency request to the sensing system, the decrease frequency request comprising a request to decrease a frequency of sending the audio data from the sensing system.

19. The method of claim 14, wherein the method comprises:
receiving an ambient noise of the operating room; and
blocking the received ambient noise.

20. The method claim 14, wherein the adjustment indication comprises a surgical step indication indicating a surgical step, and the method comprises:
receiving a first audio data associated with a first healthcare processional (HCP) role in the operating room;
receiving a second audio data associated with a second HCP role in the operating room; and
based on the surgical step indication, adjusting the AR content by allowing the first audio data associated with the first HCP and blocking the second audio data from the second HCP.
